SEQUENCE LISTING (245 sequences)

SEQ ID NO: 1 - protein, 11 aa, Homo sapiens
QPGVLIQVYEG

SEQ ID NO: 2 - protein, 36 aa, Homo sapiens
  MISC_FEATURE (1)..(9): variable feature
XXXXXXXXXLXXXXXXLXXEVXXLKEXQAKQTVCLX

SEQ ID NO: 3 - protein, 181 aa, Homo sapiens
EPPTQKPKKIVNAKKDVVNTKMFEELKSRLDTLAQEVALLKEQQALQTVCLKGTKVHMKCFLAFTQTKTFHEASEDCISRGGTLSTPQTGSEDDALTETLRQSVGNEAEIWLGLDDMAAEGTWVDMTGARIATKNWETEITAQPDGGKTEDCAVLSGAANGKWFDKRCRDQLPTICQFGIV

SEQ ID NO: 4 - DNA, 27 bp, Homo sapiens
gccctccagacggtctgcctgaagggg

SEQ ID NO: 5 - DNA, 27 bp, Homo sapiens
gttgaggcccagccagatctcggcctc

SEQ ID NO: 6 - DNA, 72 bp, Homo sapiens
  misc_feature (28)..(49): nkk is any nucleic acid sequence
gaggccgagatctggctgggcctcaacnnknnknnknnknnknnknnktgggtggacatgaccggcgcgcgc

SEQ ID NO: 7 - DNA, 27 bp, Homo sapiens
cacgatcccgaactggcagatgtaggg

SEQ ID NO: 8 - protein, 13 aa, Homo sapiens
QPGKLAQVYEGER

SEQ ID NO: 9 - protein, 13 aa, Homo sapiens
QPGVLIQAVEGER

SEQ ID NO: 10 - protein, 13 aa, Homo sapiens
APGVLIQVYEGER

SEQ ID NO: 11 - protein, 13 aa, Homo sapiens
QPGVLIQVYEGVA

SEQ ID NO: 12 - DNA, 29 bp, Homo sapiens
caccatgagccccctttggtgggggtttc

SEQ ID NO: 13 - DNA, 25 bp, Homo sapiens
ctaggcttcttctgtgtatctcttg

SEQ ID NO: 14 - DNA, 16 bp, Homo sapiens
atggccgcggcgatcg

SEQ ID NO: 15 - DNA, 19 bp, Homo sapiens
ctaatctacctcaatggtg

SEQ ID NO: 16 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 17 - protein, 20 aa, Mycobacterium tuberculosis
QPSVQIQVYQGEREIAAHNK

SEQ ID NO: 18 - protein, 13 aa, Homo sapiens
QPGVLIQVYEGER

SEQ ID NO: 19 - protein, 13 aa, Mycobacterium tuberculosis
QPSVQIQVYQGER

SEQ ID NO: 20 - DNA, 21 bp, Artificial Sequence
  Synthetic
nnknnknnknnknnknnknnk

SEQ ID NO: 21 - protein, 135 aa, Homo sapiens
LQTVCLKGTKVHMKCFLAFTQTKTFHEASEDCISRGGTLSTPQGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 22 - protein, 126 aa, Homo sapiens
NKLHAGSMGKKSGKKFFVTNHERMPFSKVKALCSELRGTVAIPRNAEENKAIQEVAKTSAFLGITDEVTEGQFMYVTGGRLTYSNWKKDEPNDHGSGEDCVTIVDNGLWNDISCQASHTAVCSFPA

SEQ ID NO: 23 - protein, 127 aa, Homo sapiens
KKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCAF

SEQ ID NO: 24 - protein, 31 aa, Equus caballus
KMFEELKSQLDSLAQEVALLKEQQALQTVCL

SEQ ID NO: 25 - protein, 31 aa, Felis catus
KMFEELKSQVDSLAQEVALLKEQQALQTVCL

SEQ ID NO: 26 - protein, 32 aa, Mus musculus
SKMFEELKNRMDVLAQEVALLKEKQALQTVCL

SEQ ID NO: 27 - protein, 31 aa, Rattus rattus
KMFEELKNRLDVLAQEVALLKEKQALQTVCL

SEQ ID NO: 28 - protein, 31 aa, Bos taurus
KMLEELKTQLDSLAQEVALLKEQQALQTVCL

SEQ ID NO: 29 - protein, 27 aa, Equus caballus
DLKTQVEKLWREVNALKEMQALQTVCL

SEQ ID NO: 30 - protein, 27 aa, Canis lupus
DLKTQVEKLWREVNALKEMQALQTVCL

SEQ ID NO: 31 - protein, 27 aa, Bos taurus
DLKTQVEKLWREVNALKEMQALQTVCL

SEQ ID NO: 32 - protein, 27 aa, Macaca mulatta
DLKTQIEKLWTEVNALKEIQALQTVCL

SEQ ID NO: 33 - protein, 28 aa, Taeniopygia guttata
DDLKTQIDKLWREVNALKEIQALQTVCL

SEQ ID NO: 34 - protein, 27 aa, Ornithorhynchus anatinus
DLKTQVEKLWREVNALKEMQALQTVCL

SEQ ID NO: 35 - protein, 27 aa, Rattus rattus
DLKSQVEKLWREVNALKEMQALQTVCL

SEQ ID NO: 36 - protein, 27 aa, Monodelphis domestica
DLKTQVEKLWREVNALKEMQALQTVCL

SEQ ID NO: 37 - protein, 28 aa, Carcharodon carcharias
DDLRNEIDKLWREVNSLKEMQALQTVCL

SEQ ID NO: 38 - protein, 31 aa, Taeniopygia guttata
KMIEDLKAMIDNISQEVALLKEKQALQTVCL

SEQ ID NO: 39 - protein, 31 aa, Gallus gallus; identical to SEQ ID NO: 38

SEQ ID NO: 40 - protein, 28 aa, Danio rerio
DDMKTQIDKLWQEVNSLKEMQALQTVCL

SEQ ID NO: 41 - protein, 28 aa, Gallus gallus
DDLKTQIDKLWREVNALKEMQALQSVCL

SEQ ID NO: 42 - protein, 28 aa, Mus musculus
DDLKSQVEKLWREVNALKEMQALQTVCL

SEQ ID NO: 43 - protein, 28 aa, Gallus gallus
DDLKTQIDKLWREVNALKEMQALQSVCL

SEQ ID NO: 44 - protein, 27 aa, Tetraodon fluviatilis
DDVRSQIEKLWQEVNSLKEMQALQTVC

SEQ ID NO: 45 - protein, 27 aa, Xenopus laevis
DLKTQIDKLWREINSLKEMQALQTVCL

SEQ ID NO: 46 - protein, 28 aa, Tetraodon nigroviridis,
EELRRQVSDLAQELNILKEQQALHTVCL

SEQ ID NO: 47 - protein, 30 aa, Xenopus laevis
KMYEELKQKVQNIELEVIHLKEQQALQTIC

SEQ ID NO: 48 - protein, 31 aa, Xenopus tropicalis
KMYEDLKKKVQNIEEDVIHLKEQQALQTICL

SEQ ID NO: 49 - protein, 28 aa, Salmo salar
EELKKQIDNIVLELNLLKEQQALQSVCL

SEQ ID NO: 50 - protein, 28 aa, Danio rerio
EELKKQIDQIIQDLNLLKEQQALQTVCL

SEQ ID NO: 51 - protein, 28 aa, Tetraodon nigroviridis
EQMQKQINDIVQELNLLKEQQALQAVCL

SEQ ID NO: 52 - protein, 28 aa, Tetraodon nigroviridis
EQMQKQINDIVQELNLLKEQQALQAVCL

SEQ ID NO: 53 - protein, 58 aa, Homo sapiens
NTGLLESQLSRHDQMLSVHDIRLADMDLRFQVLETASYNGVLIWKIRDYKRRKQEAVM

SEQ ID NO: 54 - protein, 21 aa, Homo sapiens
AASERKALQTEMARIKKWLTF

SEQ ID NO: 55 - protein, 37 aa, Homo sapiens
FDMSCRSRLATLNEKLTALERRIEYIEARVTKGETLT

SEQ ID NO: 56 - protein, 49 aa, Homo sapiens
ADIYKADFQAERQAREKLAEKKELLQEQLEQLQREYSKLKASCQESARI

SEQ ID NO: 57 - protein, 71 aa, Homo sapiens
LTGSAQNIEFRTGSLGKIKLNDEDLSECLHQIQKNKEDIIELKGSAIGLPIYQLNSKLVDLERKFQGLQQT

SEQ ID NO: 58 - protein, 28 aa, Homo sapiens
LRGLRTIVTTLQDSIRKVTEENKELANE

SEQ ID NO: 59 - protein, 27 aa, Homo sapiens
VASLRQQVEALQGQVQHLQAAFSQYKK

SEQ ID NO: 60 - protein, 27 aa, Bos taurus
VNALRQRVGILEGQLQRLQNAFSQYKK

SEQ ID NO: 61 - protein, 27 aa, Rattus rattus
SAALRQQMEALNGKLQRLEAAFSRYKK

SEQ ID NO: 62 - protein, 27 aa, Bos taurus
VNALKQRVTILDGHLRRFQNAFSQYKK

SEQ ID NO: 63 - protein, 27 aa, Bos taurus
VDTLRQRMRNLEGEVQRLQNIVTQYRK

SEQ ID NO: 64 - protein, 64 aa, Homo sapiens
GSPGLKGDKGIPGDKGAKGESGLPDVASLRQQVEALQGQVQHLQAAFSQYKKVELFPGGIPHRD

SEQ ID NO: 65 - protein, 8 aa, Homo sapiens
DMAAEGTW

SEQ ID NO: 66 - protein, 10 aa, Homo sapiens
  MISC_FEATURE (3)..(9): X is any amino acid
DMXXXXXXXW

SEQ ID NO: 67 - protein, 8 aa, Homo sapiens
  MISC_FEATURE (3)..(7): X is any amino acid
DMXXXXXW

SEQ ID NO: 68 - protein, 8 aa, Homo sapiens
  MISC_FEATURE (1)..(7): X is any amino acid
XXXXXXXW

SEQ ID NO: 69 - protein, 8 aa, Homo sapiens
DMAAEGAW

SEQ ID NO: 70 - protein, 6 aa, Artificial Sequence
  Synthrtic
DMTGGL

SEQ ID NO: 71 - protein, 6 aa, Homo sapiens
  MISC_FEATURE (3)..(7): X is any amino acid
DMXXXX

SEQ ID NO: 72 - protein, 6 aa, Artificial Sequence
  Synthetic
DMTGGX

SEQ ID NO: 73 - protein, 6 aa, Homo sapiens
DMTGAR

SEQ ID NO: 74 - protein, 6 aa, Homo sapiens
  MISC_FEATURE (6)..(6): X is any amino acid
DMTGAX

SEQ ID NO: 75 - protein, 10 aa, Homo sapiens
NWETEITAQP

SEQ ID NO: 76 - protein, 10 aa, Homo sapiens
  MISC_FEATURE (3)..(8): X is any amino acid
NWXXXXXXQP

SEQ ID NO: 77 - protein, 11 aa, Homo sapiens
  MISC_FEATURE (3)..(9): X is any amino acid
NWXXXXXXXQP

SEQ ID NO: 78 - protein, 12 aa, Artificial Sequence
  synthetic
NWXXXXXXXXQP

SEQ ID NO: 79 - protein, 16 aa, Artificial Sequence
  synthetic
NWETEXXXXXXXTAQP

SEQ ID NO: 80 - protein, 10 aa, Artificial Sequence
  synthetic
NWEXXXXXXX

SEQ ID NO: 81 - protein, 16 aa, Artificial Sequence
  synthetic
NWETEXXXXXXXTTQP

SEQ ID NO: 82 - protein, 13 aa, Artificial Sequence
  synthetic
NWETXXXXXXTQP

SEQ ID NO: 83 - protein, 7 aa, Homo sapiens
DGGATEN

SEQ ID NO: 84 - protein, 10 aa, Artificial Sequence
  synthetic
DGGXXXXXEN

SEQ ID NO: 85 - protein, 11 aa, Artificial Sequence
  Synthetic
DGGXXXXXXXN

SEQ ID NO: 86 - protein, 7 aa, Mus musculus
DGGKAEN

SEQ ID NO: 87 - DNA, 62 bp, Artificial Sequence
  synthetic
ggctgggcctgaacgacatgnnknnknnknnknnknnknnktgggtggatatgactggcgcc

SEQ ID NO: 88 - DNA, 60 bp, Artificial Sequence
  synthetic
ggcggtgatctcagtttcccagttcttgtaggcgatmnnggcgccagtcatatccaccca

SEQ ID NO: 89 - DNA, 62 bp, Artificial Sequence
  synthetic
actgggaaactgagatcaccgcccaacctgatggcggcgcaaccgagaactgcgcggtcctg

SEQ ID NO: 90 - DNA, 60 bp, Artificial Sequence
  synthetic
ccctgcagcgcttgtcgaaccacttgccgttggcggcgccagacaggaccgcgcagttct

SEQ ID NO: 91 - DNA, 30 bp, Artificial Sequence
  Syntehtic
gccgagatctggctgggcctgaacgacatg

SEQ ID NO: 92 - DNA, 23 bp, Artificial Sequence
  synthetic
atccctgcagcgcttgtcgaacc

SEQ ID NO: 93 - DNA, 67 bp, Artificial Sequence
  synthetic
gctgttcgaatacgcgcgccacagcgtgggcaacgatgcgaacatctggctgggcctcaacgatatg

SEQ ID NO: 94 - DNA, 64 bp, Artificial Sequence
  synthetic
gccgccggtcatgtcgacccamnnmnnmnnmnnmnnmnnmnncatatcgttgaggcccagccag

SEQ ID NO: 95 - DNA, 89 bp, Artificial Sequence
  synthetic
tgggtcgacatgaccggcggcnnkctggcctacaagaactgggagacggagatcacgacgcaacccgacggcggcgctgccgagaactg

SEQ ID NO: 96 - DNA, 76 bp, Artificial Sequence
  synthetic
cagcgtttgtcgaaccacttgccgttggctgcgccagacagggcggcgcagttctcggcagcgccgccgtcgggtt

SEQ ID NO: 97 - DNA, 29 bp, Artificial Sequence
  synthetic
gctgttcgaatacgcgcgccacagcgtgg

SEQ ID NO: 98 - DNA, 41 bp, Artificial Sequence
  synthetic
gggcaactgatctctgcagcgtttgtcgaaccacttgccgt

SEQ ID NO: 99 - DNA, 79 bp, Artificial Sequence
  synthetic
ggctgggcctgaacgacatgnnknnknnknnknnktgggtggatatgnnknnknnknnkatcgcctacaagaactggga

SEQ ID NO: 100 - DNA, 77 bp, Artificial Sequence
  synthetic
gacaggacggcgcagttctcggttgcgccgccatcaggttgggcggtgatctcagtttcccagttcttgtaggcgat

SEQ ID NO: 101 - DNA, 63 bp, Artificial Sequence
  synthetic
atccctgcagcgcttgtcgaaccacttgccgttggcggcgccagacaggacggcgcagttctc

SEQ ID NO: 102 - DNA, 85 bp, Artificial Sequence
  synthetic
cgtctcccagttcttgtaggccagmnnmnnmnnmnncatgtcgacccamnnmnnmnnmnnmnncatatcgttgaggcccagccag

SEQ ID NO: 103 - DNA, 62 bp, Artificial Sequence
  synthetic
gcctacaagaactgggagacggagatcacgacgcaacccgacggcggcgctgccgagaactg

SEQ ID NO: 104 - DNA, 60 bp, Artificial Sequence
  Synthetic
gagatctggctgggcctcaacnnsnnsnnsnnsnnsnnsnnstgggtggacatgactggc

SEQ ID NO: 105 - DNA, 63 bp, Artificial Sequence
  Synthetic
ttgcgcggtgatctcagtctcccagttcttgtaggcgatacgcgcgccagtcatgtccaccca

SEQ ID NO: 106 - DNA, 64 bp, Artificial Sequence
  Synthetic
gactgagatcaccgcgcaacccgatggcggcnnsnnsnnsnnsnnsgagaactgcgcggtcctg

SEQ ID NO: 107 - DNA, 60 bp, Artificial Sequence
  synthetic
ccctgcagcgcttgtcgaaccacttgccgttggccgcgcctgacaggaccgcgcagttct

SEQ ID NO: 108 - DNA, 22 bp, Artificial Sequence
  synthetic
gccgagatctggctgggcctca

SEQ ID NO: 109 - DNA, 33 bp, Artificial Sequence
  synthetic
gccatggccgccttacagactgtgtgcctgaag

SEQ ID NO: 110 - DNA, 87 bp, Artificial Sequence
  synthetic
cgtctcccagttcttgtaggccaggaggccgccggtcatgtccacccamnnmnnmnnmnnmnnmnnmnngttgaggcccagccagat

SEQ ID NO: 111 - DNA, 81 bp, Artificial Sequence
  Synthetic
gcctacaagaactgggagacggagatcacgacgcaacccgacggcggcnnknnknnknnknnkgagaactgcgccgccctg

SEQ ID NO: 112 - DNA, 38 bp, Artificial Sequence
  Synthetic
cgcacctgcggccgccacaatggcaaactggcagatgt

SEQ ID NO: 113 - DNA, 78 bp, Artificial Sequence
  Synthetic
atctggctgggcctgaacgacatggccgccgagggcacctgggtggatatgaccggcgcgcgtatcgcctacaagaac

SEQ ID NO: 114 - DNA, 62 bp, Artificial Sequence
  Synthetic
ccgccatcgggttgggcmnnmnnmnnmnnmnnmnnagtttcccagttcttgtaggcgatacg

SEQ ID NO: 115 - DNA, 57 bp, Artificial Sequence
  Synthetic
gcccaacccgatggcggcnnknnknnknnknnknnkaactgcgccgtcctgtctggc

SEQ ID NO: 116 - DNA, 54 bp, Artificial Sequence
  Synthetic
cctgcagcgcttgtcgaaccacttgccgttggcggcgccagacaggacggcgca

SEQ ID NO: 117 - DNA, 60 bp, Artificial Sequence
  Synthetic
gacatggccgcggaaggcgcctgggtcgacatgaccggcggcctgctggcctacaagaac

SEQ ID NO: 118 - DNA, 61 bp, Artificial Sequence
  Synthetic
ccgccgtcgggttgggtmnnmnnmnnmnnmnnmnnggtctcccagttcttgtaggccagca

SEQ ID NO: 119 - DNA, 57 bp, Artificial Sequence
  Synthetic
acccaacccgacggcggcnnknnknnknnknnknnkaactgcgccgccctgtctggc

SEQ ID NO: 120 - DNA, 63 bp, Artificial Sequence
  Synthetic
ctgatctctgcagcgcttgtcgaaccacttgccgttggctgcgccagacagggcggcgcagtt

SEQ ID NO: 121 - DNA, 84 bp, Artificial Sequence
  Synthetic
gccagacaggacggcgcagttmnnmnnmnngccgccmnnmnnmnnmnnmnnmnnmnnmnnttcccagttcttgtaggcgatacg

SEQ ID NO: 122 - DNA, 83 bp, Artificial Sequence
  Synthetic
gccagacagggcggcgcagttmnnmnnmnngccgccmnnmnnmnnmnnmnnmnnmnnmnnctcccagttcttgtaggccagca

SEQ ID NO: 123 - DNA, 53 bp, Artificial Sequence
  Synthetic
ccgccatcgggttgggcggtgatctcagtttcccagttcttgtaggcgatacg

SEQ ID NO: 124 - DNA, 60 bp, Artificial Sequence
  Synthetic
gcccaacccgatggcggcnnknnknnknnknnknnknnkaactgcgccgtcctgtctggc

SEQ ID NO: 125 - DNA, 52 bp, Artificial Sequence
  Synthetic
ccgccgtcgggttgggtggtgatctcggtctcccagttcttgtaggccagca

SEQ ID NO: 126 - DNA, 60 bp, Artificial Sequence
  Synthentic
acccaacccgacggcggcnnknnknnknnknnknnknnkaactgcgccgccctgtctggc

SEQ ID NO: 127 - DNA, 74 bp, Artificial Sequence
  Synthetic
ctggcgcgcgtatcgcctacaagaactggnnknnknnknnknnknnkcaacccgatggcggcgccaccgagaac

SEQ ID NO: 128 - DNA, 77 bp, Artificial Sequence
  synthetic
ctggcgcgcgtatcgcctacaagaactggnnknnknnknnknnknnknnkcaacccgatggcggcgccaccgagaac

SEQ ID NO: 129 - DNA, 77 bp, Artificial Sequence; identical to SEQ ID NO: 128
  Synthetic

SEQ ID NO: 130 - DNA, 81 bp, Artificial Sequence
  Synthetic
cctgcagcgcttgtcgaaccacttgccgttggcggcgccagacaggacggcgcagttctcggtggcgccgccatcgggttg

SEQ ID NO: 131 - DNA, 77 bp, Artificial Sequence
  Synthetic
gttctcggcagcgccgccgtcgggttgmnnmnnmnnmnnmnnmnnccagttcttgtaggccagcaggccgccggtca

SEQ ID NO: 132 - protein, 4 aa, Artificial Sequence
  Synthetic
XXXX

SEQ ID NO: 133 - DNA, 80 bp, Artificial Sequence
  synthetic
gttctcggcagcgccgccgtcgggttgmnnmnnmnnmnnmnnmnnmnnccagttcttgtaggccagcaggccgccggtca

SEQ ID NO: 134 - DNA, 83 bp, Artificial Sequence
  synthetic
gttctcggcagcgccgccgtcgggttgmnnmnnmnnmnnmnnmnnmnnmnnccagttcttgtaggccagcaggccgccggtca

SEQ ID NO: 135 - DNA, 18 bp, Artificial Sequence
  synthetic
gacatggccgcggaaggc

SEQ ID NO: 136 - DNA, 89 bp, Artificial Sequence
  Synthetic
gacaggaccgcgcagttctcgccsmagwmcccsaagccgccmnngggttgmnnmnnmnnmnnmnnctcccagttcttgtaggcgatacg

SEQ ID NO: 137 - DNA, 66 bp, Artificial Sequence
  Synthetic
atccctgcagcgcttgtcgaaccacttgccgttggccgcgcctgacaggaccgcgcagttctcgcc

SEQ ID NO: 138 - DNA, 54 bp, Artificial Sequence
  Synthetic
gagcgtgggcaacgaggccgagatctggctgggcctcaacgacatggccgccga

SEQ ID NO: 139 - DNA, 70 bp, Artificial Sequence
  Synthetic
ccagttcttgtaggcgatacgcgcgccagtcatatccacccaggtgccctcggcggccatgtcgttgagg

SEQ ID NO: 140 - DNA, 74 bp, Artificial Sequence
  Synthetic
atcgcctacaagaactgggagactgrgnnknnknnknnknnknnknnkaccgcgcaacccgatggcggtgcaac

SEQ ID NO: 141 - DNA, 74 bp, Artificial Sequence
  Synthetic
cgcttgtcgaaccacttgccgttggcggcgccagacaggacggcgcagttctcggttgcaccgccatcgggttg

SEQ ID NO: 142 - DNA, 33 bp, Artificial Sequence
  Synthetic
gatccctgcagcgcttgtcgaaccacttgccgt

SEQ ID NO: 143 - DNA, 24 bp, Artificial Sequence
  Synthetic
gcagatgtagggcaactgatctct

SEQ ID NO: 144 - DNA, 22 bp, Artificial Sequence
  Synthetic
gccgagatctggctgggcctga

SEQ ID NO: 145 - DNA, 63 bp, Artificial Sequence
  Synthetic
gccgagatctggctgggcctcaacggcagcnnknnknnknnkwcctgggtggacatgactggc

SEQ ID NO: 146 - DNA, 63 bp, Artificial Sequence; identical to SEQ ID NO: 105
  Synthetic

SEQ ID NO: 147 - DNA, 64 bp, Artificial Sequence
  Synthetic
gactgagatcaccgcgcaacccgatggcggcttcggcgtgttcggcgagaactgcgcggtcctg

SEQ ID NO: 148 - DNA, 64 bp, Artificial Sequence
  Synthetic
gactgagatcaccgcgcaacccgatggcggctggggcgtgttcggcgagaactgcgcggtcctg

SEQ ID NO: 149 - DNA, 64 bp, Artificial Sequence
  Synthetic
gactgagatcaccgcgcaacccgatggcggcttcgggtacttcggcgagaactgcgcggtcctg

SEQ ID NO: 150 - DNA, 64 bp, Artificial Sequence
  Synthetic
gactgagatcaccgcgcaacccgatggcggctgggggtacttcggcgagaactgcgcggtcctg

SEQ ID NO: 151 - DNA, 64 bp, Artificial Sequence
  Synthetic
gactgagatcaccgcgcaacccgatggcggctggggcgtgtggggcgagaactgcgcggtcctg

SEQ ID NO: 152 - DNA, 72 bp, Artificial Sequence
  Synthetic
ggcaacgatgcgaacatctggctgggcctcaacnnknnknnknnknnknnknnktgggtcgacatgaccggc

SEQ ID NO: 153 - DNA, 65 bp, Artificial Sequence
  Synthetic
ggttgcgtcgtgatctccgtctcccagttcttgtaggccaggaggccgccggtcatgtcgaccca

SEQ ID NO: 154 - DNA, 69 bp, Artificial Sequence
  Synthetic
gacggagatcacgacgcaacccgacggcggcnnknnknnknnknnkgagaactgtgctgccctgtctgg

SEQ ID NO: 155 - DNA, 61 bp, Artificial Sequence
  Synthetic
ctctgcagcgcttgtcgaaccacttgccgttggctgcgccagacagggcagcacagttctc

SEQ ID NO: 156 - DNA, 39 bp, Artificial Sequence
  synthetic
atacgcgcgccacagcgtgggcaacgatgcgaacatctg

SEQ ID NO: 157 - DNA, 23 bp, Artificial Sequence
  Synthetic
atctctgcagcgcttgtcgaacc

SEQ ID NO: 158 - DNA, 62 bp, Artificial Sequence
  Synthetic
caacccgacggcggcgctgccgagaactgcgccgccctgtctggcgcagccaacggcaagtg

SEQ ID NO: 159 - DNA, 64 bp, Artificial Sequence
  Synthetic
gcagatgtagggcaactgatctctgcagcgcttgtcgaaccacttgccgttggctgcgccagac

SEQ ID NO: 160 - DNA, 66 bp, Artificial Sequence
  Synthetic
gctggcctacaagaactgggagnnknnknnknnknnkcaacccgacggcggcgcagctgagaactg

SEQ ID NO: 161 - DNA, 68 bp, Artificial Sequence
  Synthetic
gcgcttgtcgaaccacttgccmnnmnnmnngccagacagggcggcgcagttctcagctgcgccgccgt

SEQ ID NO: 162 - DNA, 46 bp, Artificial Sequence
  Synthetic
ctgggtcgacatgaccggcggcctgctggcctacaagaactgggag

SEQ ID NO: 163 - DNA, 28 bp, Artificial Sequence
  synthetic
atctctgcagcgcttgtcgaaccacttg

SEQ ID NO: 164 - DNA, 77 bp, Artificial Sequence
  Synthetic
tgggcctgaacgacatggccgccgagggcacctgggtggatatgactggcgcgcgtatcgcctacaagaactgggag

SEQ ID NO: 165 - DNA, 59 bp, Artificial Sequence
  Synthetic
gttgcgccgccatcgggttgmnnmnnmnnmnnmnnctcccagttcttgtaggcgatacg

SEQ ID NO: 166 - DNA, 73 bp, Artificial Sequence
  Synthetic
tgtagggcaattgatccctgcagcgcttgtcgaaccacttgccmnnmnnmnngccagacaggacggcgcagtt

SEQ ID NO: 167 - DNA, 31 bp, Artificial Sequence
  Synthetic
gccgagatctggctgggcctgaacgacatgg

SEQ ID NO: 168 - DNA, 44 bp, Artificial Sequence
  Synthetic
caacccgatggcggcgcaaccgagaactgcgccgtcctgtctgg

SEQ ID NO: 169 - protein, 4 aa, Artificial Sequence
  Synthetic
XXXX

SEQ ID NO: 170 - protein, 4 aa, Artificial Sequence
  Synthetic
XXXX

SEQ ID NO: 171 - protein, 5 aa, Homo sapiens
AAEGT

SEQ ID NO: 172 - protein, 5 aa, Mus musculus
AAEGA

SEQ ID NO: 173 - protein, 4 aa, Artificial Sequence
  Synthetic
XXXX

SEQ ID NO: 174 - protein, 4 aa, Artificial Sequence
  Synthetic
XXXX

SEQ ID NO: 175 - protein, 4 aa, Artificial Sequence
  synthetic
XXXX

SEQ ID NO: 176 - protein, 4 aa, Artificial Sequence
  Synthetic
XXXX

SEQ ID NO: 177 - protein, 4 aa, Artificial Sequence
  Synthetic
XXXX

SEQ ID NO: 178 - DNA, 12 bp, Artificial Sequence
  synthetic
nnknnknnknnk

SEQ ID NO: 179 - protein, 4 aa, Artificial Sequence
  Synthetic
XXXX

SEQ ID NO: 180 - DNA, 18 bp, Artificial Sequence
  Synthetic
nnknnknnknnknnknnk

SEQ ID NO: 181 - DNA, 24 bp, Artificial Sequence
  Synthetic
nnknnknnknnknnknnknnknnk

SEQ ID NO: 182 - protein, 4 aa, Artificial Sequence
  synthetic
XXXX

SEQ ID NO: 183 - protein, 4 aa, Artificial Sequence
  Synthetic
XXXX

SEQ ID NO: 184 - protein, 7 aa, Mus musculus
DGGAAEN

SEQ ID NO: 185 - protein, 7 aa, Homo sapiens
DMAAEGT

SEQ ID NO: 186 - protein, 7 aa, Mus musculus
DMAAEGA

SEQ ID NO: 187 - protein, 13 aa, Homo sapiens
TEITAQPDGGKTE

SEQ ID NO: 188 - protein, 13 aa, Mus musculus
TEITTQPDGGKAE

SEQ ID NO: 189 - protein, 13 aa, Artificial Sequence
  Synthetic
XXXXXXXXGGXXX

SEQ ID NO: 190 - protein, 6 aa, Homo sapiens
ETEITA

SEQ ID NO: 191 - protein, 6 aa, Mus musculus
ETEITT

SEQ ID NO: 192 - protein, 52 aa, Homo sapiens
EPPTQKPKKIVNAKKDVVNTKMFEELKSRLDTLAQEVALLKEQQALQTVCLK

SEQ ID NO: 193 - protein, 52 aa, Mus musculus
ESPTPKAKKAANAKKDLVSSKMFEELKNRMDVLAQEVALLKEKQALQTVCLK

SEQ ID NO: 194 - protein, 52 aa, Gallus gallus
QQNGKGRQKPAASKKDGVSLKMIEDLKAMIDNISQEVALLKEKQALQTVCLK

SEQ ID NO: 195 - protein, 52 aa, Bos taurus
ETPTPKAKKAANAKKDAVSPKMLEELKTQLDSLAQEVALLKEQQALQTVCLK

SEQ ID NO: 196 - protein, 49 aa, Oncorhynchus keta
QQTSSKKKGGKKDAENNAAIEELKKQIDNIVLELNLLKEQQALQSVCLK

SEQ ID NO: 197 - protein, 49 aa, Xenopus laevis
QQNGKKNKQNNKDVVSMKMYEDLKKKVQNIEEDVIHLKEQQALQTICLK

SEQ ID NO: 198 - protein, 48 aa, Danio rerio
EQSLTKRKNGKKESNSAAIEELKKQIDQIIQDLNLLKEQQALQTVCLK

SEQ ID NO: 199 - protein, 52 aa, Bos taurus
QTSCHASKFKARKHSKRRVKEKDGDLKTQVEKLWREVNALKEMQALQTVCLR

SEQ ID NO: 200 - protein, 38 aa, Carcharodon carcharias
KPSKSGKGKDDLRNEIDKLWREVNSLKEMQALQTVCLK

SEQ ID NO: 201 - protein, 52 aa, Artificial Sequence
  Synthetic
XXXXXXXXXXXXXXXXXXXXXXXXXLXXXXXXLXXEVXXLKEXQALQTVCLX

SEQ ID NO: 202 - DNA, 62 bp, Artificial Sequence; identical to SEQ ID NO: 87
  Synthetic

SEQ ID NO: 203 - DNA, 12 bp, Artificial Sequence
  Synthetic
nnnnnnnnnnnn

SEQ ID NO: 204 - DNA, 60 bp, Artificial Sequence
  Synthetic
ggcggtgatctcagtttcccagttcttgtaggcgatgcgggcgccagtcatatccaccca

SEQ ID NO: 205 - DNA, 62 bp, Artificial Sequence; identical to SEQ ID NO: 89
  synthetic

SEQ ID NO: 206 - DNA, 60 bp, Artificial Sequence; identical to SEQ ID NO: 90
  Synthetic

SEQ ID NO: 207 - protein, 5 aa, Homo sapiens
AAEGT

SEQ ID NO: 208 - protein, 5 aa, Mus musculus
AAEGA

SEQ ID NO: 209 - protein, 137 aa, Homo sapiens
ALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 210 - protein, 126 aa, Homo sapiens; identical to SEQ ID NO: 22

SEQ ID NO: 211 - protein, 127 aa, Homo sapiens; identical to SEQ ID NO: 23

SEQ ID NO: 212 - protein, 123 aa, Homo sapiens
KVYWFCYGMKCYYFVMDRKTWSGCKQTCQSSSLSLLKIDDEDELKFLQLLVVPSDSCWVGLSYDNKKDWAWIDNRPSKLALNTRKYNIRDRGGCMLLSKTRLDNGNCDQVFICICGKRLDKFP

SEQ ID NO: 213 - protein, 128 aa, Homo sapiens
CPVNWVEHERSCYWFSRSGKAWADADNYCRLEDAHLVVVTSWEEQLFVQHHIGPVNTWMGLHDQNGPWKWVDGTDYETGFKNWRPEQPDDWYGHGLGGGEDCAHFTDDGRWNDDVCQRPYRWVCSTEL

SEQ ID NO: 214 - protein, 147 aa, Homo sapiens
GIPKCPEDWGASSRTSLCFKLYAKGKHEKKTWFESRDFCRALGGDLASINNKEEQQTIWRLITASGSYHKLFWLGLTYGSPSEGFTWSDGSPVSYENWAYGEPNNYQNVEYCGELKGDPTMSWNDINCEHLNNWICQIQKGQTPKPD

SEQ ID NO: 215 - protein, 129 aa, Homo sapiens
DCLSGWSSYEGHCYKAFSKYKTWEDAERVCTEQAKGAHLVSIESSGEADFVAQLVTQNMKRLDFYIWIGLRVQGKVKQCNSEWSDGSSVSYENWIEAESKTCLGLEKETDFRKWVNIYCGQQNPFVCEA

SEQ ID NO: 216 - protein, 122 aa, Homo sapiens
DCPSDWSSYEGHCYKPFSEPKNWADAENFCTQQHAGGHLVSFQSSEEADFVVKLAFQTFHSIFWMGLSNVWNQCNWQWSNAAMLRYKAWAEESYCVYFKSTNNKWRSRACRMMAQFVCEFQA

SEQ ID NO: 217 - protein, 135 aa, Homo sapiens
ARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFGKWKDVPCEDKFSFVCKFKN

SEQ ID NO: 218 - protein, 123 aa, Homo sapiens
DYEILFSDETMNYADAGTYCGSRGMALVSSAMRDSTMVKAILAFTEVKGHDYWVGADNLQDGAYNFNWNDGVSLPTDSDLWSPNEPSNPQSWQLCVQIWSKYNLLDDVGCGGARRVICEKELD

SEQ ID NO: 219 - protein, 125 aa, Homo sapiens
HMKCFLAFTQTKTFHEASEDCISRGGTLSTPQTGSENDALYEYLRQSVGNEAEIWLGLNDMAAEGTWVDMTGARIAVKNWETEITAQPDGGKTENCAVLSGAANGKWFDKRCRDQLPYICQFGIV

SEQ ID NO: 220 - protein, 114 aa, Homo sapiens
GNKFFLTNGEIMTFEKVKALCVKFQRSVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 221 - protein, 112 aa, Homo sapiens
KYFMSSVRRMPLNRAKALCSELQGTVATPRNAEENRAIQNVAKDVAFLGITDQRTENVFEDLTGNRVRYTNWNEEEPNNVGSGENCVVLLTNGKWNDVPCSDSFLVVCEFSD

SEQ ID NO: 222 - protein, 115 aa, Homo sapiens
GEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 223 - protein, 114 aa, Homo sapiens
GKKFFVTNHERMPFSKVKALCSELRGTVRIPRNAEENKRIQEVAKTSAFLGITDEVTEGQFMYVTGGRLTYSNWKKDEPNDVGSGEDCVTIVDNGLWNDVSCQASHTAVCEFPA

SEQ ID NO: 224 - protein, 122 aa, Homo sapiens
GDKVFSTENGQSVNFMDTIKEMCTRAGGRNISAVPMRTPEENEATRIMASIAKEYNNVVYLGMIEDQTPGDFHYLDGASVSYTNWYPGEPRGQGKEKCVEMYTDGTWNDRGCLQVRLAVCEF

SEQ ID NO: 225 - protein, 118 aa, Homo sapiens
TKFQGHCVRHFPDRETWVDAERRCREQQSHLSSIVTPEEQEFVNKNAQDYQWGLNDRTIEGDFRWSDGHSLQFEKWRPNQPDNFFRTGEDCVVMIWHERGEWNDVPCNVQLPFTCKKG

SEQ ID NO: 226 - protein, 125 aa, Homo sapiens
SHCVALFLSPKSWTDADLACQKRPSGNLVSVLSGAEGSFVSSLVKSIGNSVSVVWIGLHDPTQGTEGEGWEWSSSDVMNVFAWERNPSTISSPGHCASLSRSTAFLRWKDVNCNVRLPVVCKFTD

SEQ ID NO: 227 - protein, 119 aa, Homo sapiens
DKCVVFSVEKEIFEDAKLFCEDKSSHLVFINTREEQQWIKKQMVGRESHWIGLTDSERENEWKWLDGTSPDVKNWKAGQPDNWGHGHGPGEDCAGLIVAGQWNDFQCEDVNNFICEKDR

SEQ ID NO: 228 - protein, 120 aa, Homo sapiens
DKCVVFSLEKEIFEDAKLFCEDKSSHLVFINSREEQQWIKKHTVGRESHWIGLTDSEQESEWKWLDGSPVDVKNWKAGQPDNWGSGHGPGEDCAGLIVAGQWNDFQCDELNNFLCEKERE

SEQ ID NO: 229 - protein, 87 aa, Homo sapiens
GNCVFMSNSQRNWHDSVTACQEVRAQLVVIKTAEEQNFLQLQTSRSNRFSWMGLSDLNQEGTWQWVDGSPLSPSFQRVWNSGEPNNS

SEQ ID NO: 230 - DNA, 546 bp, Homo sapiens
gagccaccaacccagaagcccaagaagattgtaaatgccaagaaagatgttgtgaacacaaagatgtttgaggagctcaagagccgtctggacaccctggcccaggaggtggccctgctgaaggagcagcaggccctgcagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggcgcccgcatcgcctacaagaactgggagactgagatcaccgcgcaacccgatggcggcaagaccgagaactgcgcggtcctgtcaggcgcggccaacggcaagtggttcgacaagcgctgccgcgatcagctgccctacatctgccagttcgggatcgtgtag

SEQ ID NO: 231 - DNA, 546 bp, Homo sapiens
gagtcacccactcccaaggccaagaaggctgcaaatgccaagaaagatttggtgagctcaaagatgttcgaggagctcaagaacaggatggatgtcctggcccaggaggtggccctgctgaaggagaagcaggccttacagactgtgtgcctgaagggcaccaaggtgaacttgaagtgcctcctggccttcacccaaccgaagaccttccatgaggcgagcgaggactgcatctcgcaagggggcacgctgggcaccccgcagtcagagctagagaacgaggcgctgttcgagtacgcgcgccacagcgtgggcaacgatgcgaacatctggctgggcctcaacgacatggccgcggaaggcgcctgggtggacatgaccggcggcctcctggcctacaagaactgggagacggagatcacgacgcaacccgacggcggcaaagccgagaactgcgccgccctgtctggcgcagccaacggcaagtggttcgacaagcgatgccgcgatcagttgccctacatctgccagtttgccattgtgtag

SEQ ID NO: 232 - protein, 181 aa, Homo sapiens
ESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 233 - protein, 202 aa, Homo sapiens
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 234 - protein, 202 aa, Mus musculus
MGFWGTYLLFCLFSFLSQLTAESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 235 - protein, 201 aa, Gallus gallus
MALRGACLLLCLVSLAHISVQQNGKGRQKPAASKKDGVSLKMIEDLKAMIDNISQEVALLKEKQALQTVCLKGTKIHLKCFLAFSESKTYHEASEHCISQGGTLGTPQGGEENDALYDYMRKSIGNEAEIWLGLNDMVAEGKWVDMTGSPIRYKNWETEITTQPDGGKLENCAALSGVAVGKWFDKRCKEQLPYVCQFMIV

SEQ ID NO: 236 - protein, 202 aa, Bos taurus
MELWGPCVLLCLFSLLTQVTAETPTPKAKKAANAKKDAVSPKMLEELKTQLDSLAQEVALLKEQQALQTVCLKGTKVHMKCFLAFVQAKTFHEASEDCISRGGTLGTPQTGSENDALYEYLRQSVGSEAEVWLGFNDMASEGSWVDMTGGHIAYKNWETEITAQPDGGKVENCATLSGAANGKWFDKRCRDKLPYVCQFAIV

SEQ ID NO: 237 - protein, 198 aa, Oncorhynchus keta
MRVSGVRLLFCLLLLGQSTFQQTSSKKKGGKKDAENNAAIEELKKQIDNIVLELNLLKEQQALQSVCLKGIKIIGKCFLADTAKKIYHTAYDDCIAKGGTISTPLTGDENDQLVDYVRRSIGPEEHIWLGINDMVTEGEWLDQAGTNLRFKNWETDITNQPDGGRTHNCAILSTTANGKWFDESCRVEKASVCEFNIV

SEQ ID NO: 238 - protein, 198 aa, Xenopus laevis
MEYRRACILLCLFCFVQVTLQQNGKKNKQNNKDVVSMKMYEDLKKKVQNIEEDVIHLKEQQALQTICLKGMKIYNKCFLAFNELKTYHQASDVCFAQGGTLSTPETGDENDSLYDYVRKSIGSSAEIWIGINDMATEGTWLDLTGSPISFKHWETEITTQPDGGKQENCAALSASAIGRWFDKNCKTELPFVCQFSIV

SEQ ID NO: 239 - protein, 223 aa, Danio rerio
MRDDSDKVPSLLTDYILKGCTYAEEKMDLKAVKFLLCVICLVKSSPEQSLTKRKNGKKESNSAAIEELKKQIDQIIQDLNLLKEQQALQTVCLKGFKIPGKCFLVDTVKKDFHSANDDCIAKGGILSTPMSGHENDQLQEYVQQTVGPETHIWLGVNDMIKEGEWIDLTGSPIRFKNWESEITHQPDGGRTHNCAVLSSTANGKWFDEDCRGEKASVCQFNIV

SEQ ID NO: 240 - protein, 197 aa, Bos taurus
MAKNGLVIYILVITLLLDQTSCHASKFKARKHSKRRVKEKDGDLKTQVEKLWREVNALKEMQALQTVCLRGTKFHKKCYLAAEGLKHFHEANEDCISKGGTLVVPRSADEINALRDYGKRSLPGVNDFWLGINDMVAEGKFVDINGLAISFLNWDQAQPNGGKRENCALFSQSAQGKWSDEACHSSKRYICEFTIPQ

SEQ ID NO: 241 - protein, 166 aa, Carcharodon carcharias
SKPSKSGKGKDDLRNEIDKLWREVNSLKEMQALQTVCLKGTKIHKKCYLASRGSKSYHAANEDCIAQGGTLSIPRSSDEGNSLRSYAKKSLVGARDFWIGVNDMTTEGKFVDVNGLPITYFNWDRSKPVGGTRENCVAASTSGQGKWSDDVCRSEKRYICEYLIPV

SEQ ID NO: 242 - protein, 165 aa, Artificial Sequence
  Synthetic
MELWGALLCLFSLQVTAKAKKKKDVSKMEELKQIDLAQEVLLKEQQALQTVCLKGTKIHKCFLAFTQKTFHEASEDCISQGGTLSTPQGDENDALYRSVGNEAIWLGNDMAAEGWVDMTGSIYKNWETEITQPDGGKENCAALSANGKWFDKCRDELPYVCQFIV

SEQ ID NO: 243 - DNA, 240 bp, Homo sapiens
  misc_feature (33)..(59): nnk can encode any amino acid
gaggccgagatctggctgggcctgaacgacatgnnknnknnknnknnknnktgggtggatatgactggcgcccgcatcgcctacaagaactgggaaactgagatcaccgcccaacctgatggcggcgcaaccgagaactgcgcggtcctgtctggcgccgccaacggcaagtggttcgacaagcgctgcagggatcaattgccctacatctgccagttcgggatcgtggcggcggccgca

SEQ ID NO: 244 - protein, 80 aa, Homo sapiens
  MISC_FEATURE (12)..(18): X can be any amino acid
EAEIWLGLNDMXXXXXXXWVDMTGARIAYKNWETEITAQPDGGATENCAVLSGAANGKWFDKRCRDQLPYICQFGIVAAA

SEQ ID NO: 245 - DNA, 150 bp, Homo sapiens
caggccctccagacggtctgcctgaaggggaccaaggtgcacatgaaatgctttctggccttcacccagacgaagaccttccacgaggccagcgaggactgcatctcgcgcgggggcaccctgagcacccctcagactggctgggagacc

(19) United States
(12) Patent Application Publication
(10) Pub. No.: US 2011/0028403 A1
(43) Pub. Date: Feb. 3, 2011

(54) HSP70-Based Treatment for Autoimmune Diseases and Cancer

(75) Inventors: ISABELLE CAROLINE LE POOLE, DOWNERS GROVE, IL (US); JOSEPHUS DIRK NIELAND, ARHUS C (DK); THOR LAS HOLTET, RONDE (DK)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ANAPHORE, INC.

(21) Appl. No.: 12/723,388

(22) Filed: Mar. 12, 2010

Related U.S. Application Data
(63) Continuation-in-part of application No. PCT/US2008/076266, filed on Sep. 12, 2008.
(60) Provisional application No. 60/960,022, filed on Sep. 12, 2007.
(60) Provisional application No. 61/051,720, filed on May 9, 2008.

(51) Int. Cl. A61K 38/16; C07K 7/06; C07K 14/00; C07H 21/00; C12N 15/63; C12N 5/10; C12N 5/071; A61P 17/00

(52) U.S. Cl. 514/18.6; 530/327; 530/350; 514/21.2; 536/23.4; 435/320.1; 435/325

(57) ABSTRACT

A non-natural HSP70 activating region that activates dendritic cells. Polypeptides that bind to the HSP70 activating region can be used to treat autoimmune diseases, such as vitiligo, by binding to HSP70 and preventing HSP70 form activating dendritic cells. The HSP70 binders can be constructed in the form of fusions proteins with a trimerizing structural element that may associate to form a trimeric complex. Pharmaceutical compositions and methods for treating vitiligo using the HSP70 binding proteins, fusion proteins and complexes.

### CROSS REFERENCE TO RELATED APPLICATION

This application is a continuation-in-part of International Application No. PCT/US/2008/076266, filed Sep. 12, 2008 which claims the benefit of U.S. provisional patent application 60/960,022, filed Sep. 12, 2007 and U.S. provisional patent application 61/051,720, filed May 9, 2008, each of which is incorporated by reference herein in its entirety.

### SEQUENCE LISTING STATEMENT

The sequence listing is filed in this application in electronic format only and is incorporated by reference herein. The sequence listing text file “10-______.SequenceListing.txt” was created on ______, and is ______ bytes in size.

### BACKGROUND OF THE INVENTION

1. Field of the Invention

The invention is related to treating autoimmune diseases, such as vitiligo. In particular, the invention is related to human HSP70 protein that activates dendritic cells, peptides that bind the HSP70 protein, and methods of using the peptides to treat an autoimmune disease that is precipitated by HSP70, such as vitiligo, and cancers, such as melanoma.

2. Description of Related Art

Vitiligo is a skin disorder whose main symptom is progressive depigmentation of the skin. This disease strikes 1% of the world population, or approximately three million people in the United States alone. A common cause of depigmentation is reduced melanogenesis by existing melanocytes. In vitiligo however, depigmentation is caused by the loss of melanocytes from the basal layer of the epidermis.

Only a subset of individuals has a genetic propensity to develop vitiligo. This is reflected by the existence of intrinsic abnormalities in vitiligo melanocytes, including dilated endoplasmic reticulum profiles and abnormal melanosome compartmentalization. These abnormalities may render vitiligo patients increasingly sensitive to several forms of stress. Stress is considered a precipitating factor for vitiligo. Known stressors including bleaching phenols, UV irradiation and mechanical injury will invoke a Koebner phenomenon (i.e., the tendency of several skin conditions to affect areas subjected to injury). Patients themselves consider stress, either emotional or physical, to be a primary cause of their disease. The role of stress is further supported by the existence of “occupational vitiligo” where a subset of individuals will develop vitiligo following exposure to bleaching phenols in the workplace.

T-cell infiltrates have been consistently observed in the perilesional skin of expanding lesions from patients with generalized vitiligo, which is the most common form accounting for greater than 90% of vitiligo cases. Tumor cells isolated from vitiligo skin are cytotoxic towards autologous melanocytes. These findings indicate that vitiligo can be regarded as a T-cell mediated autoimmune disease that precipitates under stress.

Cells under stress will halt mainstream protein synthesis while inducing heat shock protein and/or glucose regulated protein synthesis (Welch 1993; Kiang and Tsokos, 1998). Stress proteins will bind to preexisting cellular proteins, preventing their degradation and thereby avoiding cellular apoptosis. It is well established that T-cell derived stress protein fractions can initiate immune responses specific to the proteins and peptides they chaperone and thus, to the cells from which they are derived. Therefore, tumor derived stress protein fractions can evoke anti-tumor immune reactivity. HSP70 is a rather unique stress protein in this regard because inducible HSP70 is secreted from live cells to serve as a chaperokine (functioning as a chaperone as well as a cytokine) (Asea et al, 2000). Exocytosis of HSP70 containing vesicles is thought to occur in response to activation of the sympathetic nervous system, ultimately leading to an increase in intracellular calcium as a signal for exocytosis for several cell types (Johnson and Fleshner, 2006). In this setting dendritic cells (DCs) are provided with antigenic peptides from live cells that can be processed and presented to T-cells in draining lymph nodes and simultaneously are activated by HSP70 which enable them to initiate an immune response. In this respect, HSP70 can stimulate the proliferation as well as the cytotoxicity of natural killer (NK) cells (Multhof et al, 1999), induce maturation and type-1 polarizing cytokine production by DCs (Wang et al, 2002), stimulate cross priming of T-cells by DCs (Kammerer et al, 2002). Most importantly, HSP70 was shown to break T-cell tolerance and induce autoimmunity in mice (Millar et al, 2003). Interestingly, an elevated surface expression of HSP70 on circulating lymphocytes was recently reported for vitiligo patients (Frediani et al, 2005). It appears therefore that among stress proteins, HSP70 is the prime contributor to an induction of immune reactivity to chaperoned proteins.

The HSP70 family is composed of at least 11 highly related genes on chromosomes 1, 5, 6, 9, 11, 14 and 21 in humans, encoding in part constitutively expressed and in part inducible proteins (Tavaria et al, 1996). The common denominator among family members is that expression of the gene product is induced by elevated temperatures (heat shock) and that the proteins have an approximate molecular weight of 70 kDa (66-78 kDa) (Tavaria et al, 1996). Most family members serve as molecular chaperones. In this function HSP70 family members will facilitate folding of nascent proteins, bind polypeptides and translocate mature proteins (Gething and Sambrook 1992). The loci encoding individual members of the HSP70 family have been named HSPA1 through HSPA9 (with both HSPA1 and HSPA2 are subclassified to multiple members). The localization of individual gene products will vary from nuclear/cytoplasmic (A1 also known as HSP72 or Hsp70i, and A8 also known as HSP73 or HSC70) to ER (5, also known as BiP or GRP78) and mitochondrial (9, also known as GRP75 or PBP74) (Tavaria et al, 1996). The chaperokine function appears to be assigned mainly to inducible HSPA1A (Johnson and Fleshner, 2006). Due to evolutionary conservation of the genes protecting cells against the physiological consequences of heat shock, homologues of this family of proteins can be found across the plant and animal kingdom.

The constitutive form of HSP70, HSPA8, reroutes cytosolic proteins otherwise destined for proteasomal degradation to the lysosome. Proteins rerouted for lysosomal degradation are linearized by a lysosomal membrane complex involving HSP70, then transferred to lysosomal associate membrane protein-2a (LAMP-2a) molecules forming a pore in the lysosomal membrane. Once inside the lysosome proteins again encounter HSP70 (lyHSP70), possibly to safeguard entering resident lysosomal proteins from inadvertent degradation. In rheumatoid arthritis, autoimmune reactivity has been assigned in part to the process whereby HSP70 chaperones proteins (including MHC class II proteins) into lysosomes. HSP70 safeguards lysosomal integrity, protecting against conditions of oxidative stress (Nylandsted et al, 2004). HSP70 present in the lysosomal membrane (facing the cytoplasm) also serves as a docking protein carrying responsibility (at least in part) for fusion of lysosomes with membranous accumulations and cytotosolics proteins in a process termed autophagy. Autophagy serves to recycle surplus intracellular molecules and structures. Disrupted autophagy may also occur in vitiligo, as supported by membranous inclusions observed in vitiligo melanocytes (Le Poole et al, 2000). Consequently, HSP70 and its co-chaperones (particularly CHIP) appear to be gatekeepers defining the proportion of proteins to undergo proteasomal degradation and enter the MHC class I route of antigen presentation, or lysosomal degradation. In cells expressing MHC class II molecules, the lysosomes are a source of peptides to be presented in the context of such MHC class II molecules. HSP70 is therefore responsible for segregation of class I and class II destinations.

Resident tissue cells can also express MHC class II molecules under exceptional circumstances. For melanocytes, these circumstances are found in melanoma and in vitiligo (Le Poole et al, 2003). Melanocytic cells harbour melanosomes as an equivalent to lysosomes in other cell types. Melanosomes engage in melanosome-phagosome fusion (Le Poole et al, 1993b; Le Poole et al, 2004). Mutations in HSP70 have been implicated in disruption of the endosomal/lysosomal compartment. The presence of HSP70 on or in melanosomes, potentially involved in trafficking of melanosomal proteins has not been investigated to date. Yet the exceptional immunogenicity of melanosomes can likely be ascribed, at least in part, to melanocyte specific melanosomal proteins presented to the immune system in the context of MHC class II molecules by vitiliginous melanocytes (Wang et al, 1999). Also, the HSP70 associated with melanosomes may be externalized during melanosome transfer, potentially affecting antigen uptake, processing and presentation by DCs.

Several surface receptors for HSP70-peptide complexes have been identified on immunocytes, including the LDL-receptor-related protein2/α2-macroglobulin CD91 (Basu et al, 2001), scavenger receptors LOX-1 (Delneste et al, 2002), CD94 (Gross et al, 2003), SR-A (Berwyn et al, 2003), and Toll-like receptors 2 and 4 (Asea et al, 2002) and CD40 (Becker et al, 2002). The relationship between anti-tumor immunity and autoimmunity to melanocytic cells in melanoma versus vitiligo (Das et al, 2001; Turk et al, 2002; Houghton and Guevara-Patino, 2004; Engelhom et al, 2006) has pointed to the involvement of heat shock proteins in vitiligo after HSPs were implicated in anti-tumor immunity (Srivastava and Udono, 1994; Castelli et al, 2004). Therefore, blocking HSP70 from perpetuating an immune response to melanocytes can benefit patients with vitiligo. Stressed melanocytes can activate dendritic cells (DC) in vitiligo via release of HSP70 by stressed melanocytes thereby inducing the expression of apoptosis inducing molecules (e.g. TRAIL). Furthermore, activated dendritic cells have cytotoxic activity after activation and can kill melanocytes, which increases the levels of HSP70 in the microenvironment.

The standard method of care for vitiligo includes prescription of topical hydrocortisone as an immunosuppressive treatment, followed by PUVA therapy to provide both immunosuppression and a melanogenic stimulus, both with limited success. Results using pseudocatalase to supplement existing melanocyte antioxidants have been disappointing. A major drawback for the development of effective treatment modalities has been the erroneous perception of an existing lesion as disease. Therefore, patients physicians, and pharmaceutical companies are looking for means to achieve repigmentation rather than aiming to interfere with depigmentation. This is an important distinction to make because a vitiligo lesion is most analogous to a scar that is left when a wound has healed.

Accordingly, the inventors have identified a need in the art to halt progression of disease by eliminating a main instigator of anti-melanocyte immunity.

### SUMMARY OF THE INVENTION

In one aspect, the invention is directed to a polypeptide having a non-natural fragment of human HSP70 activating region comprising QPGVLIQVYEG [SEQ ID NO: 1].

In another aspect, the invention is directed to a fusion protein having a trimerizing domain and at least one polypeptide that binds to QPGVLIQVYEG. The peptide may be a C-Type Lectin Like Domain (CLTD) having a loop region comprising a polypeptide sequence that binds QPGVLIQVYEG. Also, the fusion protein may have a first polypeptide that binds QPGVLIQVYEG that is positioned at one of the N-terminus and the C-terminus of the trimerizing domain and a second polypeptide that binds QPGVLIQVYEG positioned at the other of the N-terminus and the C-terminus of the trimerizing domain. One or both of the first and second polypeptides may be a C-Type Lectin Like Domain (CLTD) having a loop region comprising the polypeptide sequence that binds to QPGVLIQVYEG. The trimerizing domain may be a tetranectin trimerizing structural element. The fusion proteins may associate to form a trimeric complex.

In a further aspect, the invention is directed to a pharmaceutical composition having a peptide that binds to the HSP70 activating region and a pharmaceutically acceptable excipient. The composition can be used to treat a patient suffering from vitiligo or other autoimmune disease precipitated by HSP70.

Various further aspects of the invention include a method of preventing the activation of a dendritic cell by HSP70. The method includes contacting tissue containing the dendritic cells and cells expressing HSP70 with the a peptide having the HSP70 activating region. The peptides may be in the form of fusion proteins and trimeric complexes.

Another aspect of the invention includes a fusion protein of a trimerizing domain and an HSP70 polypeptide comprising QPGVLIQVYEG. Three fusion proteins may be in the form of a trimeric complex. The proteins and complexes may be used to activate a dendritic cell and treat melanoma.

### BRIEF DESCRIPTION OF THE FIGURES

FIG. 1 is a graph depicting results of experiments shows that human HSP70 and HSP70 mutant 10 in contrast toHSP70 mutant 5, 6 and 8, can mediate depigmentation in mice with TRP-2 induced Vitiligo phenotype (Vit mice).

FIG. 2 shows western blot analysis of the expression of HSP70i by COS cells 48 hrs after transfection.

FIG. 3 shows that depgimentation in Vit mice is accelerated in response to HSP70.

FIG. 4 shows depigmentation of Vit mice six weeks following the final gene gun vaccination.

FIG. 5 shows that ventral gene gun vaccination induced depigmentation progressing to the backs of the Vit mice.

FIG. 6 shows an alignment of the amino acid sequences of ten CTLDs of known 3D-structure. The sequence locations of main secondary structure elements are indicated above each sequence, labelled in sequential numerical order as “αN”, denoting a α-helix number N, and “βM”, denoting β-strand number M. The four cysteine residues involved in the formation of the two conserved disulfide bridges of CTLDs are indicated and enumerated in the Figure as “C_{I}”, “C_{II}”, “C_{III}” and “C_{IV}” respectively. The two conserved disulfide bridges are C_{I}-C_{IV} and C_{II}-C_{III}, respectively. The ten C-type lectins are hTN: human tetranectin, MBP: mannose binding protein; SP-D: surfactant protein D; LY49A: NK receptor LY49A; H1-ASR: H1 subunit of the asialoglycoprotein receptor; MMR-4: macrophage mannose receptor domain 4; IX-A and IX-B: coagulation factors IX/X-binding protein domain A and B. respectively; Lit: lithostatine; TU14: tunicate C-type lectin.

FIG. 7 depicts the three dimensional structure (ribbon format) for human tetranectin, depicting the secondary structural features of the protein. The structure was solved in the Ca²⁺-bound faun.

FIG. 8A depicts the three dimensional overlay structures of the CTLDs for human tetranectin (HTN) and several tetranectin homologues, including human mannose binding protein (MBP), rat mannose binding protein-C (MBP-C), human surfactant protein D, rat mannose binding protein-A (MBP-A), and rat surfactant protein A. The CTLD overlay structures were generated using Swiss PDB Viewer DeepView v. 4.0.1 for Macintosh using the three-dimensional structure of human tetranectin as a template. FIG. 8B shows the corresponding amino acid sequences of the CTLDS for human tetranectin and the tetranectin homologues depicted in FIG. 8A. In FIG. B, 1HUP=human mannose binding protein, 1BV4A=rat mannose binding protein, 2GGUA=human surfactant protein D, 1KXOA=rat mannose binding protein A, 1R13=rat surfactant protein A.

FIG. 9A depicts the three dimensional overlay structures of the CTLDs for human tetranectin (HTN) and several tetranectin homologues, including human pancreatitis-associated protein, human dendritic cell-specific ICAM-3-grabbing non-integrin 2 (DC-SIGNR), rat aggrecan, mouse scavenger receptor, and human scavenger receptor. The CTLD overlay structures were generated using Swiss PDB Viewer DeepView v. 4.0.1 for Macintosh using the three-dimensional structure of human tetranectin as a template. FIG. 9B shows the corresponding amino acid sequences of the CTLDS for human tetranectin and the tetranectin homologues depicted in FIG. 9A. In FIG. 6B, 1TDQB=rat aggrecan, 1UV0A=human pancreatitis-associated protein, 2OX8A=human scavenger receptor, 2OX9A=mouse scavenger receptor, and 1SL6A=human DC-SIGNR)

FIG. 10 depicts an alignment of the nucleotide and amino acid sequences of the coding regions of the mature forms of human and murine tetranectin with an indication of known secondary structural elements.

FIG. 11 depicts an alignment of several C-type lectin domains from tetranectins isolated from human (Swissprot P05452), mouse (Swissprot P43025), chicken (Swissprot Q9DDD4), bovine (Swissprot Q2KIS7), Atlantic salmon (Swissprot B5XCV4), frog (Swissprot Q5I0R9), zebrafish (GenBank XP_{—}701303), and related CTLD homologues isolated from cartilage of cattle (Swissprot u22298) and reef shark (Swissprot p26258).

FIG. 12 shows the PCR strategy for creating randomized loops in a CTLD.

FIG. 13 shows the DNA and amino acid sequence of the human tetranectin CTLD modified to contain restriction sites for cloning, indicating the Ca2+ binding sites. Restriction sites are underscored with solid lines. Loops are underlined with dashed lines. Calcium coordinating residues are in bold italics and include Site 1: D116, E120, G147, E150, N151; Site 2: Q143, D145, E150, D165. The CTLD domain starts at amino acid A45 in bold (i.e. ALQTVCL . . . ). Changes to the native tetranectin (TNCTLD) base sequence are shown in lower case. The restriction sites were created using silent mutations that did not alter the native amino acid sequence.

FIG. 14 depicts a non-limiting strategy for lengthening and introducing randomization in a CTLD loop region.

FIG. 15 shows alignment of the amino acid sequences of the trimerizing structural element of the tetranectin protein family. Amino acid sequences (one letter code) corresponding to residue E1 to K52 comprising exon 1, exon 2 and the first three residues of exon 3 of human tetranectin (SEQ ID NO: 192). Sequences include murine tetranectin (SEQ ID NO: 193); chicken (SEQ ID NO: 194), bovine (SEQ ID NO: 195), Atlantic salmon (SEQ ID NO: 196), frog (SEQ ID NO: 197), zebrafish (SEQ ID NO: 198) tetranectin homologous protein isolated from reefshark cartilage (SEQ ID NO: 199) and tetranectin homologous protein isolated from bovine cartilage (SEQ ID NO: 200). Residues at a and d positions in the heptad repeats are listed in boldface. The listed consensus sequence (SEQ ID NO: 201) of the tetranectin protein family trimerising structural element comprise the residues present at a and d positions in the heptad repeats shown in the figure in addition to the other conserved residues of the region. “*” denotes an aliphatic hydrophobic residue. Residues corresponding to exon 2 and the first three residues of exon 3 of human tetranectin (V17-K52) are underlined.

### DETAILED DESCRIPTION

A bibliography at the end of this Detailed Description is provided for complete citation of the literature cited herein. Each of the references, in the bibliography or as cited throughout the specification, are incorporated by reference in their entirety.

In one aspect, the invention is directed to non-natural HSP70 polypeptides that activate dendritic cells (DC). The polypeptides can be used to generate binding agents that bind to the DC activating region in human HSP70 so that immune activation can be modulated. In autoimmune diseases like vitiligo, blocking the DC activating region should be able to block disease progression. Accordingly, in one aspect, the invention is directed to methods for treating vitiligo by reducing or preventing the HSP70 induced activation of dendritic cells.

In another aspect, the invention is directed to fusion proteins of a trimerizing domain and a polypeptide that binds to the human HSP70 domain that activates dendritic cells (“HSP70 activating region”). The trimerizing domain can be associated with other similar fusion proteins to provide a stable, non-immunogenic composition for use in treating vitiligo.

In another aspect, the invention relates generally to a combinatorial polypeptide library comprising polypeptide members having a C-type lectin domain (CTLD) with a randomized loop region, in which the randomized loop region has been modified from the native sequence of the CTLD.

Before defining these and other aspects of the invention in further detail, a number of terms are defined. Unless a particular definition for a term is provided herein, the terms and phrases used throughout this disclosure should be taken to have the meaning as commonly understood in the art. Also, as used in this specification and the appended claims, the singular forms “a,” “an,” and “the” include plural referents unless the context clearly dictates otherwise.

The term “binding member”, as used herein, refers to a member of a pair of molecules which have binding specificity for one another. The members of a binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus the members of the pair have the property of binding specifically to each other.

When referring to a binding pair, such as ligand/receptor, antibody/antigen, or other binding pair, binding is measured in a binding reaction which is determinative of the presence of a member of a binding pair in a heterogeneous population of another member of the binding pair. Under designated conditions, “specific binding” occurs when one member of the binding pair binds to another member of the binding pair in a heterologous population and does not bind in a significant amount to other proteins or polypeptides present in the sample. Specific binding can be measured using the methods described herein, including Biacore and ELISA.

As used herein, the term “trimerizing domain” means an amino acid sequence that comprises the functionality to associate with two other amino acid sequences, forming a “trimer”. The trimerizing domain can associate with another trimerizing domain of identical amino acid sequence (a homotrimer), or with trimerizing domains of different amino acid sequence (a heterotrimer). Such an interaction may be caused by covalent bonds between the components of the trimerizing domains as well as by hydrogen bond forces, hydrophobic forces, van der Waals forces and salt bridges. The trimerizing effect of trimerizing domain is caused by a coiled coil structure that interacts with the coiled coil structure of two other trimerizing domains to form a triple alpha helical coiled coil trimer that is stable even at relatively high temperatures. In various embodiments, for example, a trimerizing domain based upon a tetranectin structural element (described below), the complex is stable at least 60° C., for example in some embodiments at least 70° C.

Certain non-limiting examples of trimerizing domains include the tetranectin trimerizing structural element (“TTSE”), the mannose binding protein trimerizing domain, and the collecting neck region, and the like. The “tetranectin trimerizing structural element” or “TTSE” as used herein comprises amino acids 22-49, 50, 51 or 52 of the tetranectin protein (SEQ ID NO: 21).

The trimerizing domain of a polypeptide of the invention can be derived from tetranectin as described in U.S. Patent Application Publication No. 2007/0154901 ('901 Application), which is incorporated by reference in its entirety. The term TTSE is also intended to embrace variants of a TTSE of a naturally occurring member of the tetranectin family of proteins, variants which have been modified in the amino acid sequence without adversely affecting, to any substantial degree, the capability of the TTSE to form alpha helical coiled coil trimers. Thus, the trimeric polypeptide according to the invention can comprise a TTSE as a trimerizing domain, which comprises a sequence having at least 68% amino acid sequence identity with the sequence of SEQ ID NO: 22, more particularly at least 75% identity, at least 87% identity or at least 92% identity with SEQ ID NO: 22. In accordance herewith, the cysteine residue No. 50 of the TTSE (SEQ ID NO: 21) may advantageously be mutagenized to serine, threonine, methionine or to any other amino acid residue in order to avoid formation of an unwanted inter-chain disulphide bridge, which can lead to unwanted multimerization. In a particular embodiment, the trimerizing domain is a polypeptide of SEQ ID NO: 22 which a consensus sequence of a the tetranectin family trimerizing structural element as more fully described in US2007/00154901.

The mature human tetranectin single chain polypeptide sequence is provided herein as SEQ ID NO: 3. Examples of a tetranectin trimerizing domain include the amino acids 17 to 49, 17 to 50, 17 to 51 and 17-52 of SEQ ID NO: 3, which represent the amino acids encoded by exon 2 of the human tetranectin gene, and optionally the first one, two or three amino acids encoded by exon 3 of the gene. Other examples include amino acids 1 to 49, 1 to 50, 1 to 51 and 1 to 52, which represents all of exons 1 and 2, and optionally the first one, two or three amino acids encoded by exon 3 of the gene. Alternatively, only a part of the amino acid sequence encoded by exon 1 is included in the trimerizing domain. In particular, the N-terminus of the trimerizing domain may begin at any of residues 1, 2, 3, 4, 5, 6, 7, 8, 9, 10, 11, 12, 13, 14, 15, 16 and 17 of SEQ ID NO: 23. In particular embodiments, the N terminus is I10 or V17 and the C-terminus is Q47, T48, V49, C(S)50, L51 or K52 (numbering according to SEQ ID NO: 23). See PCT US09/60271, which is incorporated by reference herein in its entirety.

Another example of a trimerizing domain is disclosed in WO 95/31540 (incorporated herein in its entirety), which describes polypeptides comprising a collectin neck region. Trimers can then be made under appropriate conditions with three polypeptides comprising the collectin neck region amino acid sequence.

Another example of a trimerizing domain is Mannose Binding Protein C trimerizing domain (MBP-C). This trimerizing domain can oligomerize even further and create higher order multimeric complexes.

Other examples of a MBP trimerizing domain is described in PCT Application Serial No. US08/76266, published as WO 2009/036349, which is incorporated by reference in its entirety. This trimerizing domain can oligomerize even further and create higher order multimeric complexes.

The terms “C-type lectin-like protein” and “C-type lectin” are used to refer to any protein present in, or encoded in the genomes of, any eukaryotic species, which protein contains one or more CTLDs or one or more domains belonging to a subgroup of CTLDs, the CRDs, which bind carbohydrate ligands. The definition specifically includes membrane attached C-type lectin-like proteins and C-type lectins, “soluble” C-type lectin-like proteins and C-type lectins lacking a functional transmembrane domain and variant C-type lectin-like proteins and C-type lectins in which one or more amino acid residues have been altered in vivo by glycosylation or any other post-synthetic modification, as well as any product that is obtained by chemical modification of C-type lectin-like proteins and C-type lectins.

The CTLD contains approximately 120 amino acid residues and, characteristically, contains two or three intra-chain disulfide bridges. Although the primary sequences of CTLDs from different proteins share relatively low amino acid sequence homology, the secondary and tertiary structures of a number of CTLDs are similar, resulting in a highly conserved three dimensional structure, in which the structural variability is essentially confined to the CTLD loop-region. The CTLD loop region, which typically contains up to five loops, plays a role in ligand and calcium binding. Several CTLDs contain either one or two binding sites for calcium and most of the side chains which interact with calcium are located in the loop-region.

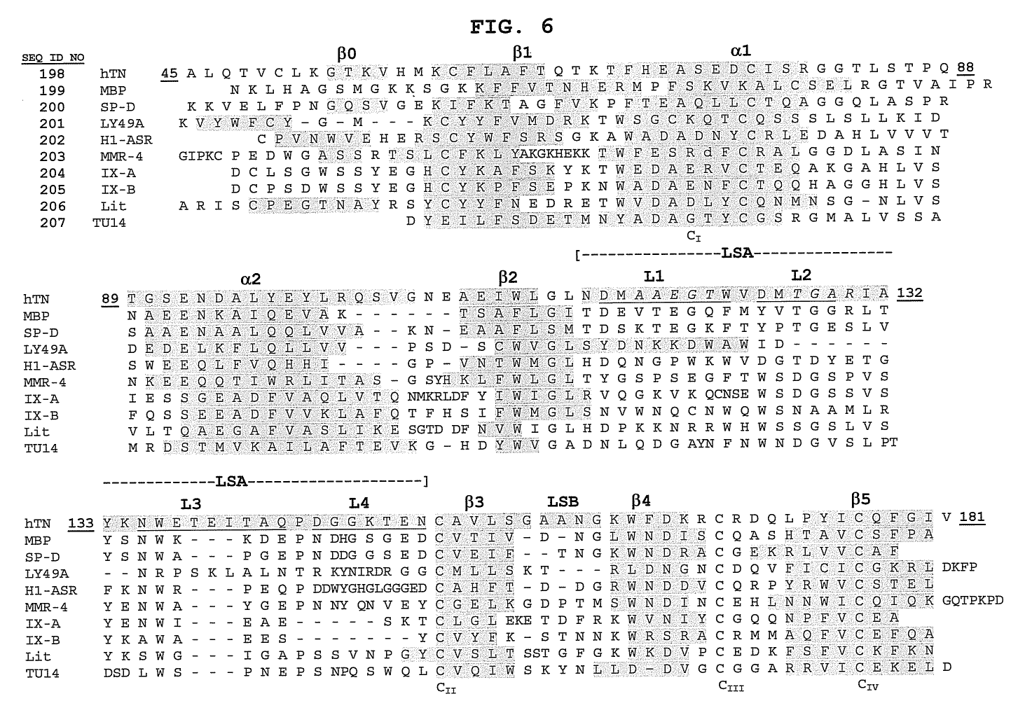

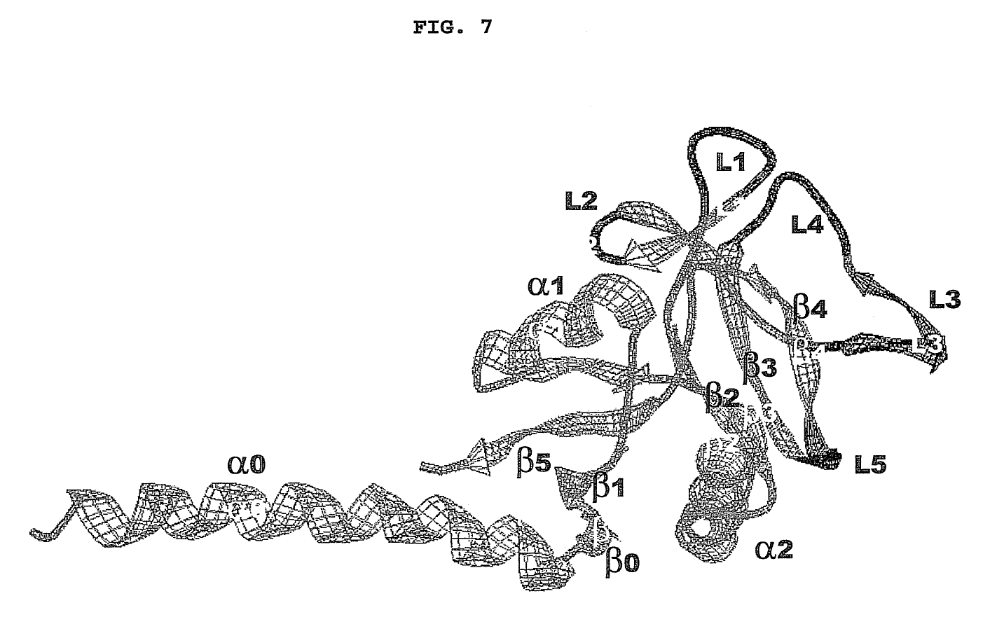

As mentioned, the loop region of any CTLD can be identified using structural and/or sequence-based analyses based on the existing sequence information for any single structurally characterized CTLD or any combination of structurally characterized CTLDs. For example, the location of the loop region of any uncharacterized CTLD can be identified by aligning a prospective CTLD sequence with the group of structure-characterized CTLDs presented in FIG. 6. The sequence alignments shown in FIG. 6 were strictly elucidated from actual three dimensional structure data. Given that the polypeptide segments of corresponding structural elements of the framework also exhibit strong amino acid sequence similarities, FIG. 6 provides a set of direct sequence-structure signatures, which can readily be inferred from the sequence alignment. As shown in FIG. 6, the loop region (LSA and LSB) is flanked by segments corresponding to the β2-, β3-, and β4-strands (loops 1-4 of LSA typically fall between the β2 and β3 strands of the canonical CTLD and loop 5 of LSB is typically located between β3 and β4 of the CTLD). The β2-, β3-, and β4-strands can be identified by identification of their respective consensus sequences (published in US Patent Application Publication 2007/0275393). The loop region of the prospective CTLD can be identified by aligning the sequence of the prospective CTLD with the sequence shown in FIG. 6 and assigning approximate locations of framework structural elements as guided by the sequence alignment, i.e., identifying the β2-, β3-, and β4-strands, adjusting the alignment to ensure precise alignment of the four canonical cysteine residues involved in the formation of the two conserved disulfide bridges (C_{I}-C_{IV} and C_{II}-C_{III}, in FIG. 6) invariably found in all CTLDs characterized thus far. Furthermore, the loop regions of a prosective CTLD can be identified using known protein structure modeling programs, such as Swiss PDB Viewer DeepView v. 4.0.1 for Macintosh, by aligning the sequence of prospective CTLD with any of the CTLD sequences in FIG. 6. Other protein modeling programs that can be used in the same manner are known in the art and available for public use, for example, MODELLER and Selvita SPMP 2.0 (See Sali A, Blundell T L. (1993) Comparative protein modelling by satisfaction of spatial restraints. J. Mol. Biol. 234, 779-815; Marti-Renom M A, Stuart A, Fiser A, Sánchez R, Melo F, Sali A. (2000) Comparative protein structure modeling of genes and genomes. Annu. Rev. Biophys. Biomol. Struct. 29, 291-325; Fiser A, Sali A. (2003) Modeller: generation and refinement of homology-based protein structure models. Methods Enzymol. 374:461-91).

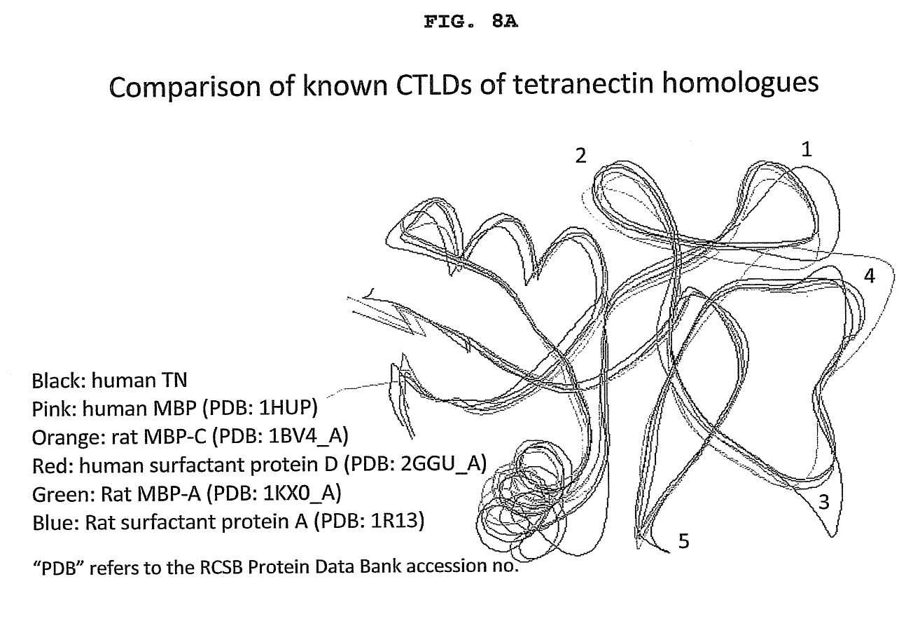

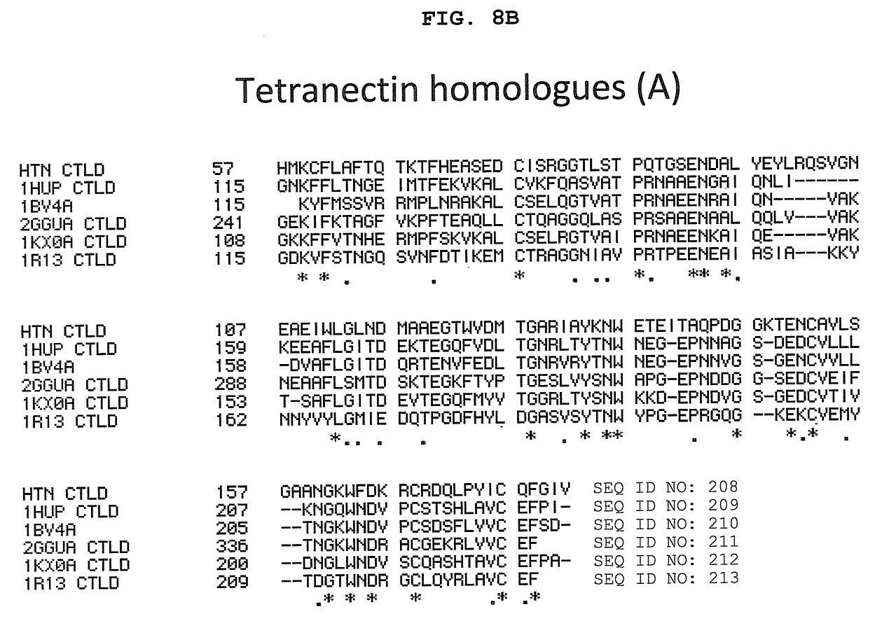

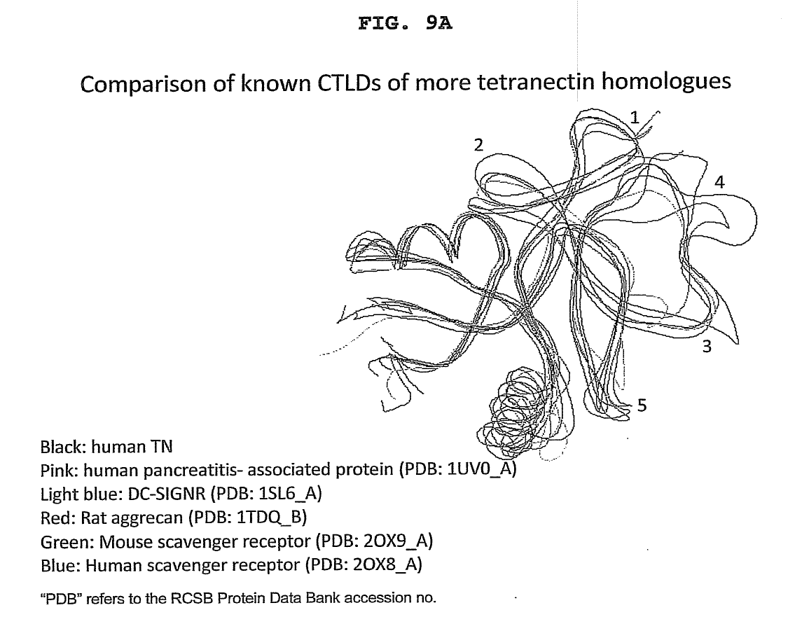

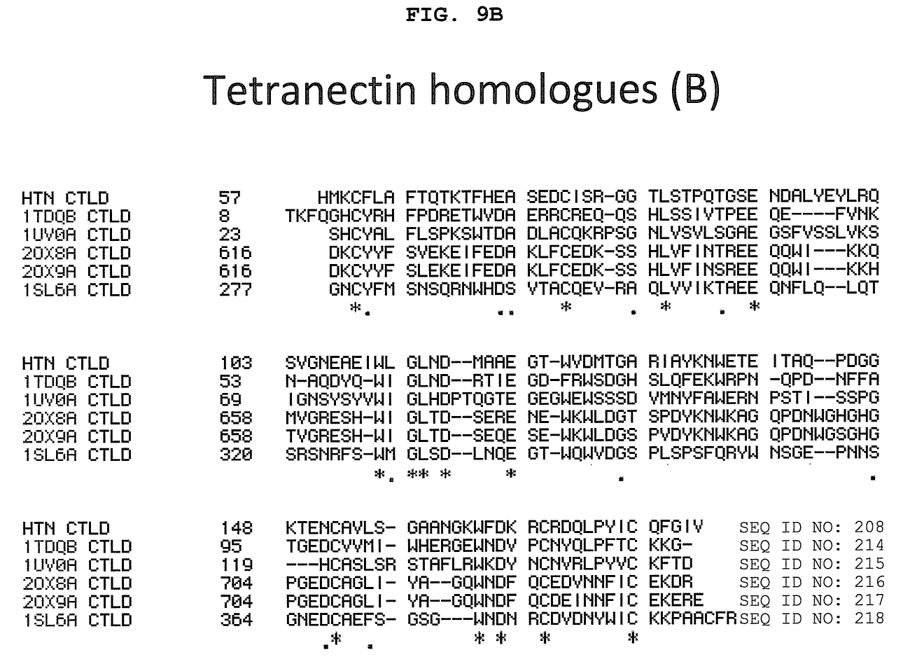

In the CTLD three-dimensional structure, the conserved secondary and tertiary structural elements form a compact scaffold for a number of loops, which in the present context collectively are referred to as the “loop-region,” protruding out from the core. The primary structure of the loop region of the CTLDs is organized into two segments, loop segment A (LSA) and loop segment B (LSB). LSA represents the long polypeptide segment connecting β2 and β3 which often lacks regular secondary structure and contains up to four loops. LSB represents the polypeptide segment connecting the β-strands β3 and β4. A schematic of a CTLD, including the loop region, is shown in FIGS. 7-9. Residues in LSA, together with single residues in β4, have been shown to specify the Ca²⁺- and ligand-binding sites of several CTLDs, including that of tetranectin. For example, mutagenesis studies, involving substitution of a single or a few residues, have shown that changes in binding specificity, Ca²⁺-sensitivity and/or affinity can be accommodated by CTLD domains (Weis and Drickamer (1996), Chiba et al. (1999), Graversen et al. (2000)).

The invention may also incorporate the use of tetranectin. Tetranectin is a trimeric glycoprotein (Holtet et al. (1997), Nielsen et al. (1997)) which has been isolated from human plasma and found to be present in the extracellular matrix in certain tissues. Tetranectin is known to bind calcium, complex polysaccharides, plasminogen, fibrinogen/fibrin, and apolipoprotein (a). The interaction with plasminogen and apolipoprotein (a) is mediated by the kringle 4-protein domain therein. This interaction is known to be sensitive to calcium and to derivatives of the amino acid lysine (Graversen et al. (1998)).

A number of CLTDs are known, including the following non-limiting examples: tetranectin, lithostatin, mouse macrophage galactose lectin, Kupffer cell receptor, chicken neurocan, perlucin, asialoglycoprotein receptor, cartilage proteoglycan core protein, IgE Fc receptor, pancreatitis-associated protein, mouse macrophage receptor, Natural Killer group, stem cell growth factor, factor IX/X binding protein, mannose binding protein, bovine conglutinin, bovine CL43, collectin liver 1, surfactant protein A, surfactant protein D, e-selectin, tunicate c-type lectin, CD94 NK receptor domain, LY49A NK receptor domain, chicken hepatic lectin, trout c-type lectin, HIV gp 120-binding c-type lectin, dendritic cell immunoreceptor DC-Sign, and many snake venom proteins

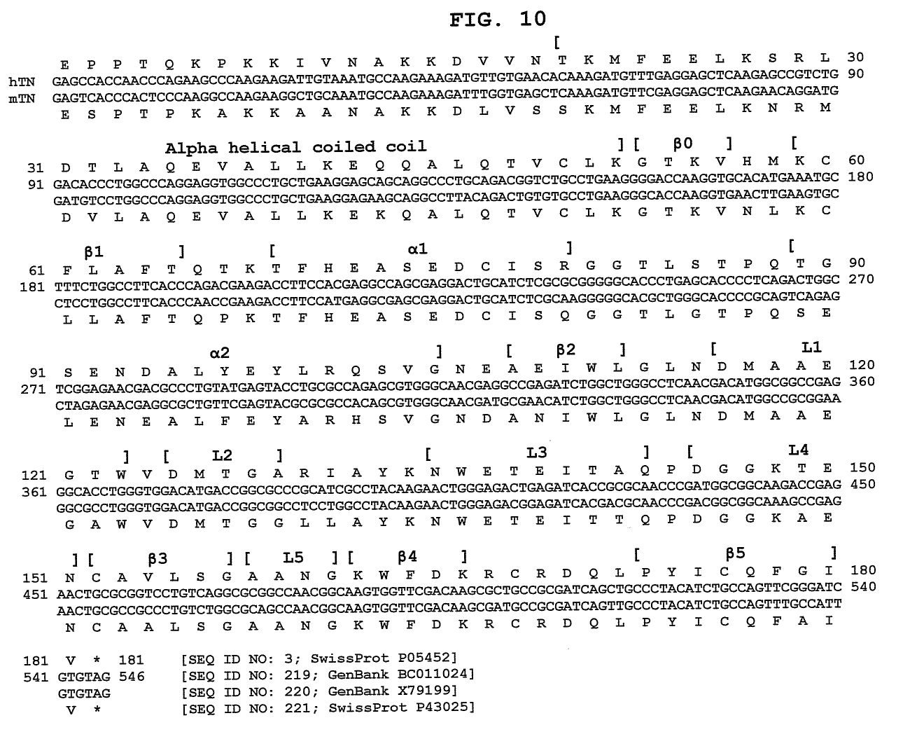

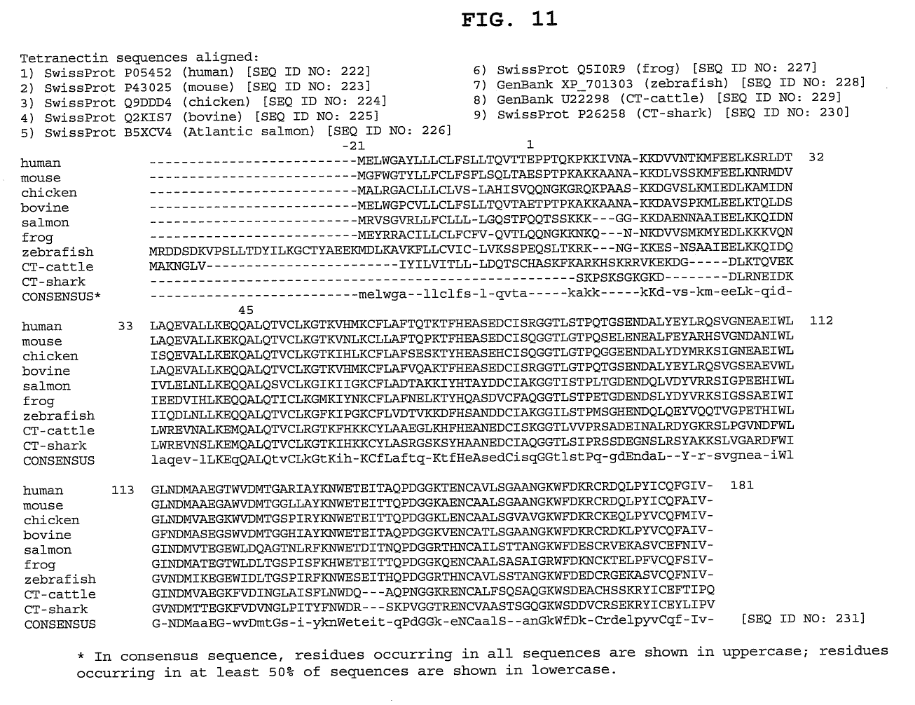

In particular embodiments, the CTLD sequence is a human or murine tetranectin CTLD sequence that is modified according to the invention. FIG. 10 shows the alignment of the nucleic acid and polypeptide sequences of human and mouse tetranectin CTLDs. In other embodiments, the CTLD is from a variety of peptides, for example, those shown in FIG. 11, which shows an alignment of several CTLDs from tetranectins isolated from human (Swissprot P05452), mouse (Swissprot P43025), chicken (Swissprot Q9DDD4), bovine (Swissprot Q2KIS7), Atlantic salmon (Swissprot B5XCV4), frog (Swissprot Q5I0R9), zebrafish (GenBank XP_{—}701303), and related CTLD homologues isolated from cartilage of cattle (Swissprot u22298) and reef shark (Swissprot p26258).

The terms “amino acid,” “amino acids,” and “amino acid residues” refer to all naturally occurring L-amino acids, as well as non-naturally occurring amino acids. This definition is meant to include norleucine, ornithine, and homocysteine. The naturally occurring L-amino acids can be classified according to the chemical composition and properties of their side chains. They are broadly classified into two groups, charged and uncharged. Each of these groups is divided into subgroups to classify the amino acids more accurately: A. Charged Amino Acids—(A.1. Acidic Residues): Asp, Glu; (A.2. Basic Residues): Lys, Arg, His, Orn; B. Uncharged Amino Acids—(B.1. Hydrophilic Residues): Ser, Thr, Asn, Gln; (B.2. Aliphatic Residues): Gly, Ala, Val, Leu, Ile, Nle; (B.3. Non-polar Residues): Cys, Met, Pro, Hcy; (B.4. Aromatic Residues): Phe, Tyr, Trp.

A “non-natural amino acid ” or “non-naturally occurring amino acid” refers to an amino acid that is not one of the 20 common amino acids including, for example, amino acids that occur by modification (e.g. post-translational modifications) of a naturally encoded amino acid (including but not limited to, the 20 common amino acids or pyrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

Many of the unnatural amino acids suitable for use in the present invention are commercially available, e.g., from Sigma (USA) or Aldrich (Milwaukee, Wis., USA). Those that are not commercially available are optionally synthesized as provided herein or as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al., (1995) J. Med. Chem., 38, 4660-4669; King, F. E. & Kidd, D. A. A. (1949) A New Synthesis of Glutamine and of .gamma.-Dipeptides of Glutamic Acid from Phthylated Intermediates. J. Chem. Soc., 3315-3319; Friedman, O. M. & Chatterrji, R. (1959) Synthesis of Derivatives of Glutamine as Model Substrates for Anti-Tumor Agents. J. Am. Chem. Soc. 81, 3750-3752; Craig, J. C. et al. (1988) Absolute Configuration of the Enantiomers of 7-Chloro-4[[4-(diethylamino)-1-methylbutyl]amino]quinoline (Chloroquine). J. Org. Chem. 53, 1167-1170; Azoulay, M., Vilmont, M. & Frappier, F. (1991) Glutamine analogues as Potential Antimalarials, Eur. J. Med. Chem. 26, 201-5; Koskinen, A. M. P. & Rapoport, H. (1989) Synthesis of 4-Substituted Prolines as Conformationally Constrained Amino Acid Analogues. J. Org. Chem. 54, 1859-1866; Christie, B. D. & Rapoport, H. (1985) Synthesis of Optically Pure Pipecolates from L-Asparagine. Application to the Total Synthesis of (+)-Apovincamine through Amino Acid Decarbonylation and Iminium Ion Cyclization. J. Org. Chem. 1989: 1859-1866; Barton et al., (1987) Synthesis of Novel a-Amino-Acids and Derivatives Using Radical Chemistry: Synthesis of L-and D-α-Amino-Adipic Acids, L-α-aminopimelic Acid and Appropriate Unsaturated Derivatives. Tetrahedron Lett. 43: 4297-4308; and, Subasinghe et al., (1992) Quisqualic acid analogues: synthesis of beta-heterocyclic 2-aminopropanoic acid derivatives and their activity at a novel quisqualate-sensitized site. J. Med. Chem. 35: 4602-7. See also, US 2004/0198637 and US 2005/0170404, each of which is incorporated by reference herein in their entirety.

The terms “amino acid modification(s)” and “modification(s)” refer to amino acid substitutions, deletions or insertions or any combinations thereof in an amino acid sequence relative to the native sequence. Substitutional variants herein are those that have at least one amino acid residue in a native CTLD sequence removed and a different amino acid inserted in its place at the same position. The substitutions may be single, where only one amino acid in the molecule has been substituted, or they may be multiple, where two or more amino acids have been substituted in the same molecule. Specific reference to more than one amino acid substitution in a CTLD refers to multiple substitutions in which each individual amino acid substitution can occur at any amino acid position within the CTLD, including consecutive and non-consecutive amino acid positions. Likewise, specific reference to more than one amino acid insertion or deletion in a CTLD refers to multiple insertions or deletions in which each individual amino acid insertion or deletion can occur at any amino acid position within the CTLD, including consecutive and non-consecutive amino acid positions.

The terms “nucleic acid molecule encoding”, “DNA sequence encoding”, and “DNA encoding” refer to the order or sequence of deoxyribonucleotides along a strand of deoxyribonucleic acid. The order of these deoxyribonucleotides detellnines the order of amino acids along the polypeptide chain. The DNA sequence thus encodes the amino acid sequence.

The terms “randomize,” “randomizing” and “randomized” as well as any similar terms used in any context to identify randomized polypeptide or nucleic acid sequences, refer to ensembles of polypeptide or nucleic acid sequences or segments, in which the amino acid residue or nucleotide at one or more sequence positions may differ between different members of the ensemble of polypeptides or nucleic acids, such that the amino acid residue or nucleotide occurring at each such sequence position may belong to a set of amino acid residues or nucleotides that may include all possible amino acid residues or nucleotides or any restricted subset thereof. The terms are often used to refer to ensembles in which the number of possible amino acid residues or nucleotides is the same for each member of the ensemble, but may also be used to refer to such ensembles in which the number of possible amino acid residues or nucleotides in each member of the ensemble may be any integer number within an appropriate range of integer numbers.

The terms “modulate” or “modulating” when used with reference to either the binding affinity of a CTLD to plasminogen, metal (e.g., Mg²⁺, Ca²⁺, Zn²⁺, Mn²⁺, etc.) or any other target molecule, such as the HSP70 activating region, refer to a change in the binding affinity of a modified CTLD polypeptide to either plasminogen or metal ion or target molecule relative to the binding affinity of the native (unmodified) CTLD polypeptide. Thus, “modulating” includes increasing binding affinity, decreasing binding affinity, and/or abolishing or abrogating binding affinity (although not to the exclusion of the specific recitation of the terms “abolishing” or “abrogating” plasminogen, metal ion, or target molecule binding activity).

Turning now to the invention in more detail, in one aspect the invention is directed to a polypeptide comprising non-natural fragment of human HSP70 comprising QPGVLIQVYEG [SEQ ID NO:1]. This peptide represents the activating region in human HSP70 for activating dendritic cells. Activated dendritic cells have cytotoxic and T cell stimulatory activity after activation and are able to kill melanocytes, thereby increasing direct or indirect the levels of HSP70 in the environment.

Non-natural fragments of human HSP70 include portions of human HSP70 that are less than full length HSP70. In particular, non-natural fragments of HSP70 include, but are not limited to, polypeptide sequences that are 11, 13, 15, 20, 25, 30, 40, 50, 75, 100, 125, and 150 amino acids in length. Non-natural fragments also include natural HSP70 that has been truncated at the N or C terminus, or having one or more deletions of amino acids between the termini. The non-natural fragments of the invention include the HSP70 activating region of SEQ ID NO:1. Such fragments are not naturally expressed by any species as a truncated wild-type sequence and may be isolated and purified as readily known in the art.

In another aspect, the invention is directed to polypeptides that bind the HSP70 activating domain. In this aspect, the invention is directed to a peptide, a protein or a fusion protein comprising a trimerizing domain and at least one polypeptide binding member that binds to the HSP70 activating region. In accordance with the invention, the binding member may either be linked to the N- or the C-terminal amino acid residue of the trimerising domain. Also, in certain embodiments it may be advantageous to link a binding member to both the N-terminal and the C-terminal of the trimerizing domain.

In another aspect, a polypeptide binding member is contained in the loop region of a CTLD. The polypeptide may be a naturally or non-naturally occurring sequence. In this aspect the sequence is contained in a loop region of a CLTD, and the CTLD is fused to a trimerizing domain at the N-terminus or C-terminus of the domain either directly or through the appropriate linker. Also, the fusion protein of the invention may include a second CLTD domain, fused at the other of the N-terminus and C-terminus. In a variation of this aspect, the fusion protein includes a binding member at one of the termini of the trimerizing domain and a CLTD at the other termini. One, two or three of the fusion proteins can be part of a trimeric complex containing up to six specific binding members for the HSP70 activating region.

In another embodiment, the binding member comprises an antibody or an antibody fragment. In the present context, the term “antibody” is used to describe an immunoglobulin whether natural or partly or wholly synthetically produced. As antibodies can be modified in a number of ways, the term “antibody” should be construed as covering any specific binding member or substance having a binding domain specificity for QPGVLIQVYEG [SEQ ID NO:1]. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. The term also covers any polypeptide or protein having a binding domain which is, or is homologous to, an antibody binding domain, e.g. antibody mimics. These can be derived from natural sources, or they may be partly or wholly synthetically produced. Examples of antibodies are the immunoglobulin isotypes and their isotypic subclasses; fragments which comprise an antigen binding domain such as Fab, Fab′, F(ab′)₂, scFv, Fv, dAb, Fd; and diabodies.

In another aspect the invention relates to a trimeric complex of three fusion proteins, each of the three fusion proteins comprising a trimerizing domain and at least one polypeptide that binds to the HSP70 activating region. In an embodiment, the trimeric complex comprises a fusion protein having a trimerizing domain selected from a tetranectin trimerizing structural element, a mannose binding protein (MBP) trimerizing domain, a collecting neck region and others. The trimeric complex can be comprised of any of the fusion proteins of the invention wherein the fusion proteins of the trimeric complex comprise trimerizing domains that are able to associate with each other to form a trimer. Accordingly, in some embodiments, the trimeric complex is a homotrimeric complex comprised of fusion proteins having the same amino acid sequences. In other embodiments, the trimeric complex is a heterotrimeric complex comprised of fusion proteins having different amino acid sequences such as, for example, different trimerizing domains, and/or different polypeptides that bind to the HSP70 activating region.

It was previously determined that the mycobacterial HSP70 sequence QPSVQIQVYQGEREIAAHNK [SEQ ID NO: 17] (aa 407-426) can activate dendritic cells (Wang et al, J. Immunology, 174(6):3306 (2005)). These results were then used to identify the region of human HSP70 that is responsible for activating dendritic cells. The QPGVLIQVYEGER [SEQ ID NO: 18] sequence of human HSP70 was chosen for analysis. This sequence is homologous to a portion of the mycobacterial sequence described above (QPSVQIQVYQGER [SEQ ID NO: 19]; aa 407-419), which is a portion of the 20-mer peptide that was reported to be an immunostimulatory region. Id. As reported, the alanine substitution of the first four amino terminal amino acids of this peptide significantly inhibited immune stimulation. Id.

As described in the Examples below, a number of mutations were introduced in the 13-mer human sequence based on interspecies homology. Four mutants were generated and the vectors containing these sequences were tested in a Vitiligo mouse model. Since mutation of the final two amino acids (i.e. mutant 10) has no effect on depigmentation, it is evident that these amino acids were not necessary for mediating depigmentation in vitiligo. Accordingly polypeptide QPGVLIQVYEG [SEQ ID NO: 1] of the invention is identified as the HSP70 activating region responsible for mediating depigmentation in vitiligo.

Other aspects of the invention are directed to preventing the activation of dendritic cells by inhibiting the interaction of the stimulatory part of hsp70 with the cell through competitive binding of an antagonistic peptide to hsp70 peptide.

Another aspect is directed to treating a stress related autoimmune disease precipitated by HSP70, such as vitiligo, by administering to a patient suffering from such disease an effective amount of a polypeptide that binds to the HSP70 activating region. The polypeptide can be part of a fusion protein along with a trimerizing domain, and may be part of a trimeric complex as described. For treating vitiligo, the preferred route of administration in topical, in a pharmaceutical acceptable delivery vehicle.

In another aspect of the invention, the HSP70 activating region can be used to activate dendritic cells. In this aspect the domain is fused to a trimerizing domain to produce a fusion protein. As described above, the fusion protein may be part of a trimeric complex, and it may include, a CTLD loop region that has grafted into it the HSP70 activating region.

The antigens recognized by T cells infiltrating vitiligo skin were previously identified as prime target antigens for T cells infiltrating melanoma tumors (Das et al, 2001). These antigens are expressed in the melanosome, which bears functional resemblance to lysosomes in other cell types (Le Poole et al, 1993). The localization likely contributes to the immunogenicity of melanosomal proteins such as gp100, MART-1 and tyrosinase. A resemblance between immune reactivity in vitiligo and melanoma is supported by leukoderma observed in melanoma patients with a detectable immune response to their tumor. In fact, depigmentation is considered a positive prognostic factor for melanoma patients (Nordlund et al, 1983). Unfortunately the immune response is rarely able to clear melanoma tumors, whereas effective immunity is a hallmark of progressive vitiligo. It thus appears that vitiligo patients develop a more vigorous immune response to melanocytic cells than melanoma patients do (Garbelli et al, 2005).

The chaperone function of HSP70, supporting uptake and processing of antigens by DCs renders the molecule an ideal candidate to serve as an adjuvant in anti-tumor vaccines. DNA encoding HSP70-antigen fusion proteins has been included in vaccines to melanoma (Zhang et al, 2006). Such applications frequently make use of mycobacterial HSP70 (Chen et al, 2000). For anti-cancer vaccines, the use of xenogeneic stress proteins has the added advantage that nucleotide variations render the resulting protein increasingly immunogenic (mycobacterial and mouse HSP70 are approximately 50% homologous), whereas either version can bind peptides and proteins. Conservation of the molecule among species is further supported by the observation that murine cell lines will bind human HSP70 and vice versa (MacAry et al, 2004). Three functional domains have been assigned within the HSP70 molecule: an N-terminal ATPase domain of approximately 44 kD (˜350 aa), a roughly 18 kD peptide binding domain (˜150 aa) and a 10 kD C terminal domain (˜100 aa) apparently responsible for binding chaperone cofactors (Lehner et al, 2004). Several surface receptors for HSP70-peptide complexes have been identified on immunocytes, including the LDL-receptor-related protein2/α2-macroglobulin CD91 (Basu et al, 2001), scavenger receptors LOX-1 (Delneste et al, 2002), CD94 (Gross et al, 2003) and SR-A (Berwyn et al, 2003), Toll-like receptors 2 and 4 (Asea et al, 2002) and CD40 (Becker et al, 2002).

The relationship between anti-tumor immunity and autoimmunity to melanocytic cells in melanoma versus vitiligo has been reported, (Das et al, 2001; Turk et al, 2002; Houghton and Guevara-Patino, 2004; Engelhom et al, 2006) (Srivastava and Udono, 1994; Castelli et al, 2004). Whereas vaccines supporting the role of HSP70 in anti-tumor immunity will benefit melanoma patients, blocking HSP70 from perpetuating an immune response to melanocytes can benefit patients with vitiligo.

Several vaccines are under development to boost anti-tumor immunity in melanoma, including vaccines based on HSP70 fusion proteins (Huang et al, 2003). HSP70 or heat shock protein 70 is included in vaccines as a chaperone protein, immunogenic in its own right and functioning as an adjuvant to stimulate DC activation and T cell reactivity.

Accordingly, another aspect of the invention includes a method of treating melanoma by activating dendritic cells. The method includes contacting a dendritic cell with the fusion protein or trimeric complex. In various aspects of the invention, the molecule can be used as a vaccine for skin cancer (melanoma) or other types of cancer, or virus vaccine or as adjuvant in vaccines, alone or ligated to the antigen to which an immune response has to be generated

Other aspects of the invention are directed to nucleotide sequences, vectors and host cells for expressing the fusion proteins of the invention as further described in US 2007/0154901.

Method of identification of binding members to the HSP70 activating region

In one aspect, a binding member for the HSP70 activating region can be obtained from a random library of polypeptides by selection of members of the library that specifically bind to the HSP70 activating region. A number of systems for displaying phenotypes with putative ligand binding sites are known. These include: phage display (e.g. the filamentous phage fd [Dunn (1996), Griffiths and Duncan (1998), Marks et al. (1992)], phage lambda [Mikawa et al. (1996)]), display on eukaryotic virus (e.g. baculovirus [Ernst et al. (2000)]), cell display (e.g. display on bacterial cells [Benhar et al. (2000)], yeast cells [Boder and Wittrup (1997)], and mammalian cells [Whitehorn et al. (1995)], ribosome linked display [Schaffitzel et al. (1999)], and plasmid linked display [Gates et al. (1996)].

Also, US2007/0275393, which is incorporated herein by reference in its entirety, specifically describes a procedure for accomplishing a display system for the generation of CLTD libraries. The general procedure includes (1) identification of the location of the loop-region, by referring to the 3D structure of the CTLD of choice, if such information is available, or, if not, identification of the sequence locations of the β2, β3 and β4 strands by sequence alignment with the sequences shown in FIG. 6, as aided by the further corroboration by identification of sequence elements corresponding to the β2 and β3 consensus sequence elements and β4-strand characteristics, also disclosed above; (2) subcloning of a nucleic acid fragment encoding the CTLD of choice in a protein display vector system with or without prior insertion of endonuclease restriction sites close to the sequences encoding β2, β3 and β4; and (3) substituting the nucleic acid fragment encoding some or all of the loop-region of the CTLD of choice with randomly selected members of an ensemble consisting of a multitude of nucleic acid fragments which after insertion into the nucleic acid context encoding the receiving framework will substitute the nucleic acid fragment encoding the original loop-region polypeptide fragments with randomly selected nucleic acid fragments. Each of the cloned nucleic acid fragments, encoding a new polypeptide replacing an original loop-segment or the entire loop-region, will be decoded in the reading frame determined within its new sequence context.

A complex may be formed that functions as a homo-trimeric protein. The trimeric structure of the human tetranectin protein presents a uniquely ideal scaffold in which to construct libraries with members capable of binding the HSP70 activating region. However peptides with HSP70 binding activity must be identified first. To accomplish this, peptides with known binding activity can be used or additional new peptides identified by screening from display libraries. A number of different display systems are available, such as but not limited to phage, ribosome and yeast display.

To select for new peptides with binding activity, libraries can be constructed and initially screened for binding to the HSP70 activating region as monomeric elements, either as single monomeric CTLD domains, or individual peptides displayed on the surface of phage. Once sequences with HSP70 binding activity have been identified these sequences would subsequently be grafted on to the trimerization domain of human tetranectin to create potential protein therapeutics capable of binding the human HSP70 activating region.

Four strategies may be employed in the construction of these phage display libraries and trimerization domain constructs. The first strategy would be to construct and/or use random peptide phage display libraries. Random linear peptides and/or random peptides constructed as disulfide constrained loops would be individually displayed on the surface of phage particles and selected for binding to the HSP70 activating region through phage display “panning”. After obtaining peptide clones with HSP70 binding activity, these peptides would be grafted on to the trimerization domain of human tetranectin or into loops of the CTLD domain followed by grafting on the trimerization domain and screened for HSP70 binding activity.

A second strategy for construction of phage display libraries and trimerization domain constructs would include obtaining CTLD derived binders. Libraries can be constructed by randomizing the amino acids in one or more of the five different loops within the CTLD scaffold of human tetranectin displayed on the surface of phage. Binding to the HSP70 activating region can be selected for through phage display panning. After obtaining CTLD clones with peptide loops demonstrating HSP70 binding activity, these CTLD clones can then be grafted on to the trimerization domain of human tetranectin and screened for HSP70 binding activity.

A third strategy for construction of phage display libraries and trimerization domain constructs would include taking known sequences with binding capabilities to the HSP70 activating region and graft these directly on to the trimerization domain of human tetranectin and screen for HSP70 binding activity.

A fourth strategy includes using peptide sequences with known binding capabilities to the HSP70 activating region and first improve their binding by creating new libraries with randomized amino acids flanking the peptide or/and randomized selected internal amino acids within the peptide, followed by selection for improved binding through phage display. After obtaining binders with improved affinity, the binders of these peptides can be grafted on to the trimerization domain of human tetranectin and screening for HSP70 binding activity. In this method, initial libraries can be constructed as either free peptides displayed on the surface of phage particles, as in the first strategy (above), or as constrained loops within the CTLD scaffold as in the second strategy also discussed above. After obtaining binders with improved affinity, grafting of these peptides on to the trimerization domain of human tetranectin and screening for HSP70 binding activity would occur.

Truncated version of the trimerization domain can be used that eliminate amino acids at the N or C terminus of a trimerizing domain. For example US Patent application publication US-2010-0028995 describes a number of truncated trimerizing polypeptides derived from human tetranectin. In various examples there, the human tetranectin trimerizing polyeptpide was truncated to either eliminate up to 16 residues at the N-terminus (V17), or alter the C-terminus. C-terminal variations termed Trip V, Trip T, Trip Q and Trip K. These polyepeptides allow for unique presentation of the CTLD domains on the trimerization domain. The TripK variant is the longest construct and contains the longest and most flexible linker between the CTLD and the trimerization domain. Trip V, Trip T, Trip Q represent fusions of the CTLD molecule directly onto the trimerization module without any structural flexibility but are turning the CTLD molecule one-third going from Trip V to Trip T and from Trip T to Trip Q. This is due to the fact that each of these amino acids is in an α-helical turn and 3.2 aa are needed for a full turn. Free peptides selected for binding in the first, third and fourth strategies can be grafted onto any of above versions of the trimerization domain Resulting fusions can then be screened to see which combination of peptide and orientation gives the best activity. Peptides selected for binding constrained within the loops of the CTLD of tetranectin can be grafted on to the full length trimerization domain.

The four strategies described above are described in further detail below. Although these strategies focus on phage display, other equivalent methods of identifying polypeptides can be used.

Strategy 1

Peptide display library kits such as, but not limited to, the New England Biolabs Ph.D. Phage display Peptide Library Kits are sold commercially and can be purchased for use in selection of new and novel peptides with HSP70 binding activity. Three forms of the New England Biolabs kit are available: the Ph.D.-7 Peptide Library Kit containing linear random peptides 7 amino acids in length, with a library size of 2.8×10⁹ independent clones, the Ph.D.-C7C Disulfide Constrained Peptide Library Kit containing peptides constructed as disulfide constrained loops with random peptides 7 amino acids in length and a library size of 1.2×10⁹ independent clones, and the Ph.D.-12 Peptide Library Kit containing linear random peptides 12 amino acids in length, with a library size of 2.8×10⁹ independent clones.

Alternatively similar libraries can be constructed de novo with peptides containing random amino acids similar to these kits. For construction random nucleotides are generated using either an NNK, or NNS strategy, in which N represents an equal mixture of the four nucleic acid bases A, C, G and T. The K represents an equal mixture of either G or T, and S represents and equal mixture of either G or C. These randomized positions can be cloned onto to the Gene III protein in either a phage or phagemid display vector system. Both the NNK and the NNS strategy cover all 20 possible amino acids and one stop codon with slightly different frequencies for the encoded amino acids. Because of the limitations of bacterial transformation efficiency, library sizes generated for phage display are in the order of those started above, thus peptides containing up to seven randomized amino acids positions (NNKNNKNNKNNKNNKNNKNNK) [SEQ ID NO: 20] can be generated and yet cover the entire repertoire of theoretical combinations (20⁷=1.28×10⁹). Longer peptide libraries can be constructed using either the NNK or NNS strategy however the actual phage display library size likely will not cover all the theoretical amino acid combinations possible associated with such lengths due to the requirement for bacterial transformation.

Strategy 2

In one aspect, the invention relates to the use of a C-type lectin-like domain (CTLD) to identify polypeptides that bind to the HSP70 activating region. The variation of binding site configuration among naturally occurring CTLDs shows that their common core structure can accommodate many essentially different configurations of the ligand binding site (see, e.g., US 2007/0275393, which is incorporated by reference herein). CTLDs are therefore particularly well suited to serve as a basis for constructing new and useful protein products with desired binding properties to HSP70 activating region of interest.

For example, the CTLDs (or CTLD-based protein products) have advantages relative to antibody derivatives as each binding site in a CTLD-based protein product is harbored in a single structurally autonomous protein domain. Also, the CTLD domains are resistant to proteolysis, and neither stability nor access to the ligand-binding site is compromised by the attachment of other protein domains to the N- or C-terminus of the CTLD.

In one aspect, the invention relates generally to a combinatorial polyp eptide library comprising polypeptide members having a C-type lectin domain (CTLD) with a randomized loop region, in which the randomized loop region has been modified from the native sequence of the CTLD. The randomized loop region of the CTLD can comprise one or more amino acid modifications in at least one of the four loops in the loop segment A (LSA) of the CTLD and can further comprise one or more amino acid modifications in the loop in Loop Segment B (LSB) (also known as loop 5). The invention also relates to methods for generating and using the randomized combinatorial polypeptide libraries to identify binding partners for the HSP70 activating region. By applying standard combinatorial methods known in the chemical, recombinant protein and antibody arts, the libraries and methods of the invention allow for the generation, screening, and identification of protein products that exhibit binding specificity to the HSP70 activating region.

The variation of binding site configuration among naturally occurring CTLDs shows that their common core structure can accommodate many essentially different configurations of the ligand binding site (see, e.g., US 2007/0275393). CTLDs are therefore particularly well suited to serve as a basis for constructing such new and useful protein products with desired binding properties. Accordingly, while in one aspect the invention relates to combinatorial polypeptide libraries comprising modifications to the loop region of the CTLD (LSA and LSB), other modifications to the general CTLD core structure (i.e., the β-strands and α-helices) can be made without affecting the utility of the libraries described herein. One of skill in the art can target particular modifications in the CTLD core structure that will retain CTLD functionality. For example, based on secondary and tertiary structures of various polypeptides comprising CTLDs, hydropathy, charge (ionic), and hydrogen bonding interactions can all be taken into consideration, and appropriate substitutions made which retain CTLD function. Such modifications include conservative amino acid substitutions. In embodiments that comprise variants, such as deletion, insertion, or substitution variants in the region outside of the loop region of the CTLD, the percent identity can be as low as 50%. In other embodiments comprising such variation within the CTLD region, variants are at least 80% identical to any given CTLD sequence, or CTLD consensus sequence. In certain embodiments such variants are at least 85%, 86%, 87%, 88%, 89%, 90%, 91%, 92%, 93%, 94%, 95%, 96%, 97%, 98%, or 99%, identical to any CTLD sequence, or CTLD consensus sequence.

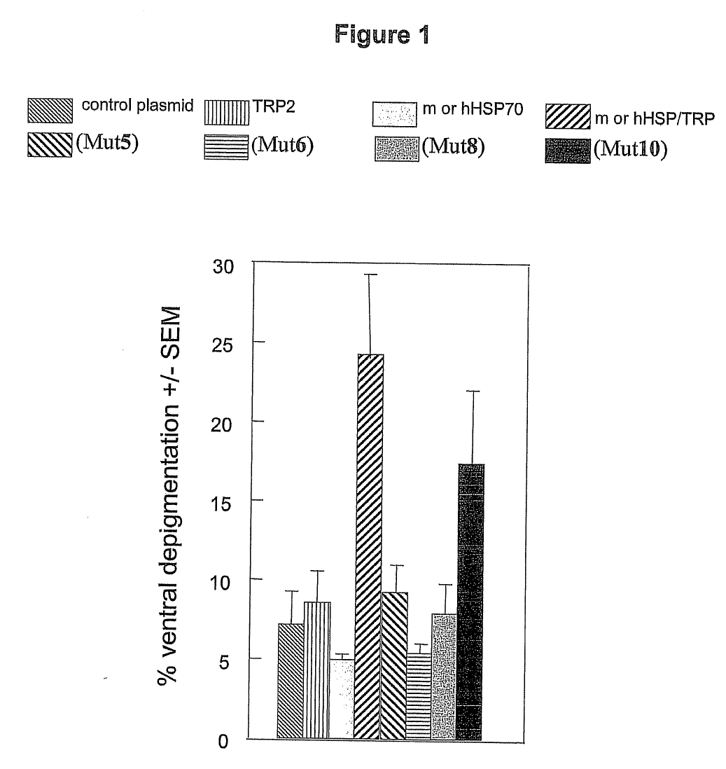

The CTLD used in the combinatorial libraries can be derived from any CTLD. Examples of suitable CTLDs are CTLDs described herein (i.e., FIGS. 6-8) and in US 2007/0275393, which is incorporated by reference herein in its entirety (i.e., FIG. 1 and Table 1) and CTLDs otherwise known in the art. In certain embodiments, the CTLD has the following secondary structure: five β-strands and two α-helices sequentially appearing in the order β1, α1, α2, β2, β3, β4, and β5, the β-strands being arranged in two anti-parallel β-sheets, one composed of β1 and β5, the other composed of β2, β3 and β4, at least two disulfide bridges, one connecting α1 and β5 and one connecting β3 and the polypeptide segment connecting β4 and β5, and a loop region containing loop segment A (LSA) and loop segment B (LSB) in which LSA connects β2 and β3, and LSB connects β3 and β4.

Thus, in a broad aspect, the invention provides a polypeptide library comprising polypeptide members that comprise a C-type lectin domain (CTLD), wherein the CTLD comprises one or more amino acid modifications in at least one of the four loops in the loop segment A (LSA) of the CTLD, and/or in the loop in loop segment B (LSB) (Loop 5). Examples of polypeptide libraries comprising polypeptides having a C-type lectin domain comprising one or more amino acid modifications in at least one of the five loops in the loop region (LSA and LSB) of the CTLD are described herein.

In certain embodiments of the polypeptide libraries, the polypeptide members have CTLDs in which one, two, three, four, or five of the CTLD loops have one or more amino acid modifications, wherein the one or more modifications include at least one amino acid insertion that extends the loop region beyond its original length. In certain of these embodiments, the one or more modifications include from 1 to about 30 amino acid insertions (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid insertions) in any single loop in the loop region (LSA and LSB). In certain of these embodiments, the one or more modifications include at least one amino acid insertion in at least two of the five loops in the loop region (e.g., two, three, or four loops in LSA or one, two, or three loops in LSA and one loop in LSB).

The polypeptides comprising a CTLD used in the polypeptide libraries of the invention can be full-length proteins or partial proteins having a CTLD, for example, the full-length amino acid sequence or partial amino acid sequence of any of the proteins described herein and otherwise known. Alternatively, the polypeptides comprising a CTLD used in the polypeptide libraries of the invention can be polypeptides comprising only CTLD sequence, for example, the amino acid sequence of any of the CTLDs described herein and otherwise known. The polypeptides comprising CTLD sequence can have additional flanking C-terminal and/or N-terminal (non-CTLD) amino acid sequence.

In certain embodiments, the polypeptide libraries comprise polypeptide members that comprise a C-type lectin domain (CTLD), wherein the CTLD comprises one or more amino acid modifications in at least one of the five loops in the loop region (LSA and LSB), wherein certain Ca²⁺ coordinating amino acids in the loop regions are retained. In other embodiments, the polypeptide libraries comprise polypeptide members that comprise a C-type lectin domain (CTLD), wherein the CTLD comprises one or more amino acid modifications in at least one of the five loops in the loop region (LSA and LSB), wherein certain amino acid(s) involved with plasminogen binding activity are eliminated.

In certain embodiments of this aspect, the polypeptide library comprises polypeptide members that comprise a C-type lectin domain (CTLD), wherein the CTLD comprises one or more amino acid modifications in regions of the CTLD that fall outside of the LSA and LSB regions. Accordingly, such modifications can be designed or randomly generated in any one or more of the beta strand and/or alpha helical regions.

The loop region of any CTLD, if not already identified or characterized, can be identified by using any variety of structural or sequence-based analysis using the existing sequence based information for any single structurally characterized CTLD or any combination of structurally characterized CTLDs. Typically, the loop regions are stretches of amino acids found between more ordered regions of the CTLD amino acid sequence (e.g., between the α-helices or β-strands), and typically have a more flexible conformation. Loop segment A (LSA) in a CTLD typically falls between the β2 and β3 strands of the canonical CTLD motif. The (LSA) contains smaller loop regions (loops 1, 2, 3, and 4), which are usually located between small beta sheet structures that provide a degree of order to the (LSA) (see, e.g., FIG. 7). CTLDs typically have a smaller loop structure (loop segment B, “LSB” or “loop 5”) located between β3 and β4.

A number of specific motifs for libraries based upon a CTLD have been described (see U.S. application Ser. No. 12/703,752, which is incorporated herein by reference). The term “1X-2 Library” refers to a combinatorial polypeptide library comprising polypeptide members that have a C-type lectin domain (CTLD) comprising amino acid modifications in at least one of the four loops in the LSA of the CTLD, wherein the amino acid modifications comprise at least two amino acid insertions in Loop 1 and random substitution of at least five amino acids within Loop 1 of the CTLD.

The term “1-2 library” refers to a combinatorial polypeptide library comprising polypeptide members that have a C-type lectin domain (CTLD) comprising amino acid modifications in at least one of the four loops in the LSA of the CTLD, wherein the amino acid modifications comprise random substitution of at least five amino acids within Loop 1 and random substitution of at least three amino acids within Loop 2.

The term “1-4 library” refers to a combinatorial polypeptide library comprising polypeptide members that have a C-type lectin domain (CTLD) comprising amino acid modifications in at least one of the four loops in the LSA of the CTLD, wherein the amino acid modifications comprise random substitution of at least seven amino acids within Loop 1, and at least one three amino acid insertions in Loop 4, and random substitution of at least two amino acids.

The term “3X library” refers to a combinatorial polypeptide library comprising polypeptide members that have a C-type lectin domain (CTLD) comprising amino acid modifications in at least one of the four loops in the LSA of the CTLD, wherein the amino acid modifications comprise a mixture of random substitution of at least six amino acids, random substitution of at least six amino acids and at least one amino acid substitution, and random substitution of at least six amino acids and at least two amino acid substitutions in Loop 3. at least one amino acid insertion in Loop 3 and random substitution of at least three amino acids within Loop 3.

The term “3-4X library” refers to a combinatorial polypeptide library comprising polypeptide members that have a C-type lectin domain (CTLD) comprising amino acid modifications in at least one of the four loops in the LSA of the CTLD, wherein the amino acid modifications comprise at least one three amino acid insertions in Loop 3 and random substitution of at least three amino acids within Loop 3 and comprise at least one three amino acid insertions in Loop 4 and random substitution of at least three amino acids within Loop 4.

The term “3-4 combo library” refers to a combinatorial polypeptide library comprising polypeptide members that have a C-type lectin domain (CTLD) comprising amino acid modifications in at least one of the four loops in the LSA of the CTLD, wherein the amino acid modifications comprise a modification that combines two loops into a single loop, wherein the two combined loops are Loop 3 and Loop 4.

The term “4 library” refers to a combinatorial polypeptide library comprising polypeptide members that have a C-type lectin domain (CTLD) comprising amino acid modifications in at least one of the four loops in the LSA of the CTLD, wherein the amino acid modifications comprise at least one four amino acid insertions in Loop 4 and random substitution of at least three amino acids within Loop 4.

The term “3-5 library” refers to a combinatorial polypeptide library comprising polypeptide members that have a C-type lectin domain (CTLD) comprising amino acid modifications in at least one of the four loops in the LSA of the CTLD, wherein the amino acid modifications comprise random substitution of at least five amino acid residues s within Loop 3 and random substitution of at least three amino acids within Loop 5.

The term “Loop 3X loop library” refers to a combinatorial polypeptide library comprising polypeptide members that have a C-type lectin domain (CTLD) comprising amino acid modifications in at least one of the four loops in the LSA of the CTLD, wherein the amino acid modifications comprise random substitution of at least one amino acid and at least six amino acid insertions.

A human tetranectin gene has been characterized, and both human and murine tetranectin cDNA clones have been isolated. The mature protein of both the human and murine tetranectin comprises 181 amino acid residues. See US Patent Application Publication 2007/0154901, which is incorporated here in its entirety. The three dimensional structures of full length recombinant human tetranectin and of the isolated tetranectin CTLD have been determined independently in two separate studies (Nielsen et al. (1997) and Kastrup et al. (1998)). Tetranectin is a two- or possibly three-domain protein, i.e. the main part of the polypeptide chain comprises the CTLD (amino acid residues Gly53 to Val181), whereas the region Leu26 to Lys52 encodes an alpha-helix governing trimerization of the protein via the formation of a homotrimeric parallel coiled coil. The polypeptide segment Glu1 to Glu25 contains the binding site for complex polysaccharides (Lys6 to Lys15) (Lorentsen et al. (2000)) and appears to contribute to stabilization of the trimeric structure (Holtet et al. (1997)). The two amino acid residues Lys148 and Glu150, localized in loop 4, and Asp165 (localised in β4) have been shown to be of critical importance for plasminogen kringle 4 binding, with residues Ile140 (in loop 3) and Lys166 and Arg167 (in β4) shown to be of importance as well (Graversen et al. (1998)). Substitution of Thr149 (in loop 4) with an aromatic residue has been shown to significantly increase affinity of tetranectin to kringle 4 and to increase affinity for plasminogen kringle 2 to a level comparable to the affinity of wild type tetranectin for kringle 4 (Graversen et al. (2000)). Trimerizable truncations of tetranectin have been described. See US 2010/0028995, filed Apr. 8, 2009, which is incorporated by reference herein in its entirety.

Analysis of the nucleotide sequence encoding the mature form of human tetranectin (FIG. 10) reveals that a recognition site for the restriction endonuclease Bgl II is found at position 326 to 331 (AGATCT), involving the encoded residues Glu109, Ile110, and Trp111 of β2, and that a recognition site for the restriction endonuclease Kas I is found at position 382 to 387 (GGCGCC), involving the encoded amino acid residues Gly128 and Ala129 (located C-terminally in loop 2). By utilizing alternate codons for naturally occurring amino acids in the tetranectin sequence, the restriction endonuclease sites Pst I (CTGCAG) and Mfe I (CAATTG) were engineered into the tetranectin coding sequence at positions 501 to 506 (CTGCCG, originally), involving the encoded amino acid residues Arg167, Cys168, and Arg169, and positions 511 to 516 (CAGCTG, originally), involving the encoded amino acid residues Gln171 and Leu172, all located between β4 and β5.

Generating randomized and optimized recombinant CTLD libraries to obtain protein products that can bind specifically to targets of interest can be performed by any technique known in the art such as, for example, oligonucleotide-directed randomization, error-prone PCR mutagenesis, DNA shuffling by random fragmentation, loop shuffling, loop walking, somatic hypermutation (see, e.g., US Patent Publication 2009/0075378, which is incorporated by reference), and other known methods in the art to create sequence diversity in order to generate molecules with optimal binding activity. (See, e.g., Stemmer, W. P., Proc Natl Acad Sci USA, (October 1994) 91:10747-751; Patrick, W. M. & Firth, A. E., Biomolecular Engineering, (2005) 22:105-112; Firth, A. E. & Patrick, W. M., Bioinformatics, (2005) 21(15):3314-3315; and Lutz S. & Patrick, W. M., Curr. Opin. Biotechnol., (2004) 15:291-297).

The human tetranectin CTLD shown in FIG. 6 contains five loops, which can be altered to confer binding of the CTLD to different proteins targets. Random amino acid sequences can be placed in one or more of these loops to create libraries from which CTLD domains with the desired binding properties can be selected. Construction of these libraries containing random peptides constrained within any or all of the 5 loops of the human tetranectin CTLD can be accomplished (but is not limited to) using either a NNK or NNS as described above in strategy 1. A single example of a method by which 7 random peptides can be inserted into loop 1 of the TN CTLD is as follows.

PCR of fragment A can be performed using the forward oligoF1 (5′-GCC CTC CAG ACG GTC TGC CTG AAG GGG-3′; SEQ ID NO:4) which binds to the N terminus of the CTLD; the reverse oligo R1 (5′-GTT GAG GCC CAG CCA GAT CTC GGC CTC-3′; SEQ ID NO:5) which binds to the DNA sequence just 5′ to loop 1. Fragment B can be created using forward oligo F2 (5′-GAG GCC GAG ATC TGG CTG GGC CTC AAC NNK NNK NNK NNK NNK NNK NNK TGG GTG GAC ATG ACC GGC GCG CGC ATC-3′; SEQ ID NO:6) and the reverse primer R2 (5′-CAC GAT CCC GAA CTG GCA GAT GTA GGG -3′; SEQ ID NO:7). The forward primer F2 has a 5′-end that is complementary to primer R1, and replaces the first seven amino acids of loop 1 with random amino acids, and contains a 3′ end which binds to last amino acid of loop 1 and the sequences 3′ of it, while the reverse primer R2 is complementary and binds to the end of the CTLD sequences. PCR can be performed using a high fidelity polymerase or taq blend and standard PCR thermocycling conditions. Fragments A and B can then be gel isolated and then combined for overlap extension PCR using the primers F1 and R2 as described above. Digestion with the restriction enzymes Bgl II and PstI can allow for isolation of the fragment containing the loops of the TN CTLD and subsequent ligation into a phage display vector (such as CANTAB 5E) containing the restriction modified CTLD shown below fused to Gene III, which is similarly digested with Bgl II and Pst I for cloning.

Modification of other loops by replacement with randomized amino acids can be similarly performed as shown above. The replacement of defined amino acids within a loop with randomized amino acids is not restricted to any specific loop, nor is it restricted to the original size of the loops. Likewise, total replacement of the loop is not required, partial replacement is possible for any of the loops. In some cases retention of some of the original amino acids within the loop, such as the calcium coordinating amino acids, may be desirable. In these cases, replacement with randomized amino acids may occur for either fewer of the amino acids within the loop to retain the calcium coordinating amino acids, or additional randomized amino acids may be added to the loop to increase the overall size of the loop yet still retain these calcium coordinating amino acids. Very large peptides can be accommodated and tested by combining loop regions such as loops 1 and 2 or loops 3 and 4 into one larger replacement loop. In addition, other CTLDs, such as but not limited to the MBL CTLD, can be used instead of the CTLD of tetranectin. Grafting of peptides into these CTLDs can occur using methods similar to those described above.

In certain embodiments, the generating and optimizing methods comprise an oligonucleotide-directed randomization (NNK or NNS) strategy for mutagenizing the loops. For example, the human tetranectin (hTN) CTLD shown in FIG. 6 and FIG. 7 contains five loops (four loops in LSA and one loop in LSB), which can be altered to confer binding of the CTLD to any target molecule(s) of interest, including the HSP70 activating region. Random amino acid sequences (generated via randomization, substitution, insertion, etc) can be introduced into one or more of these loops to create libraries from which CTLD domains with the desired binding properties can be selected. Construction of these libraries containing random peptides constrained within any or all of the five loops of the human tetranectin CTLD can be accomplished using either a NNK or NNS as described herein. These libraries can comprise further amino acid modifications that are introduced in regions of the CTLD that are outside of the LSA or LSB regions (e.g., the α-helices and/or β-strands). The following procedure describes a non-limiting, illustrative example of a method by which seven random peptides can be inserted into loop 1 of the hTN CTLD.

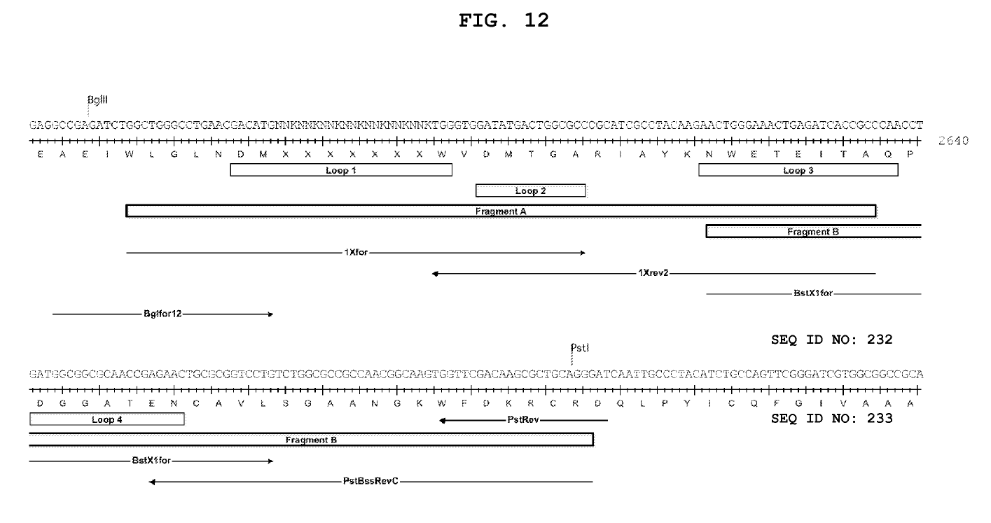

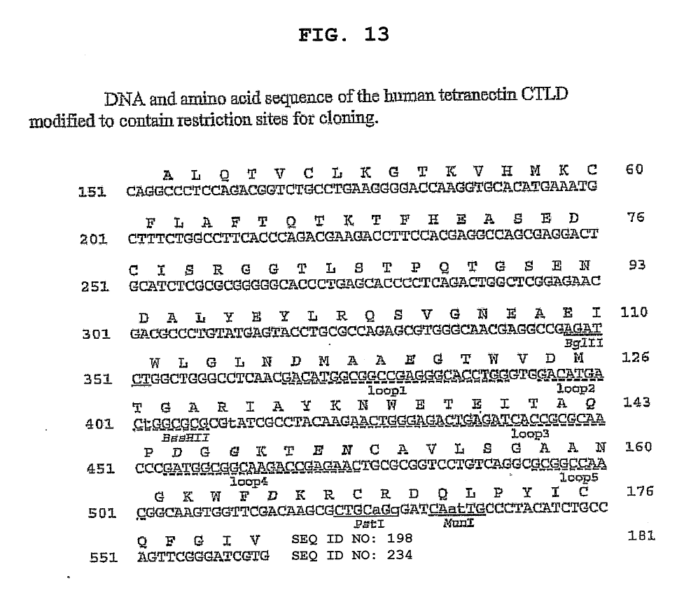

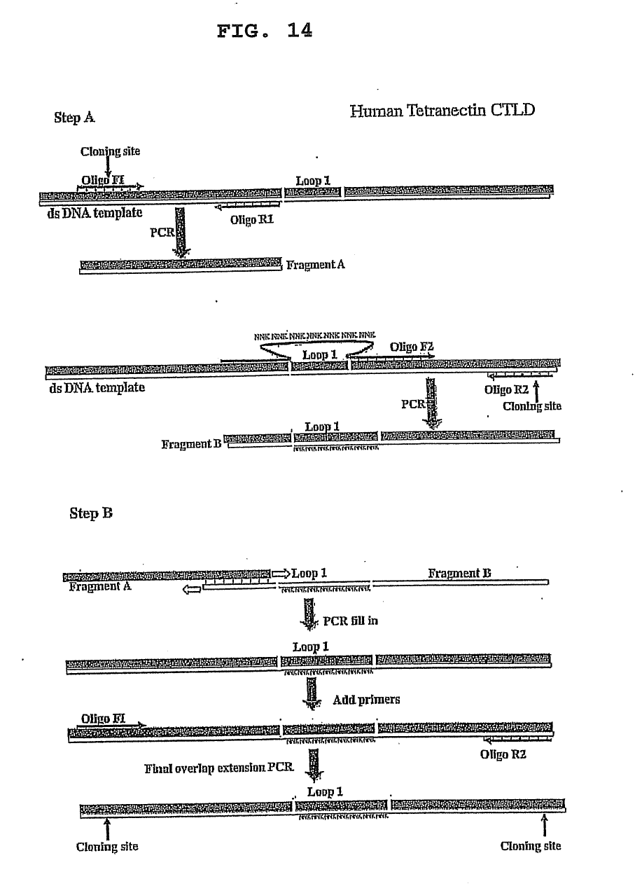

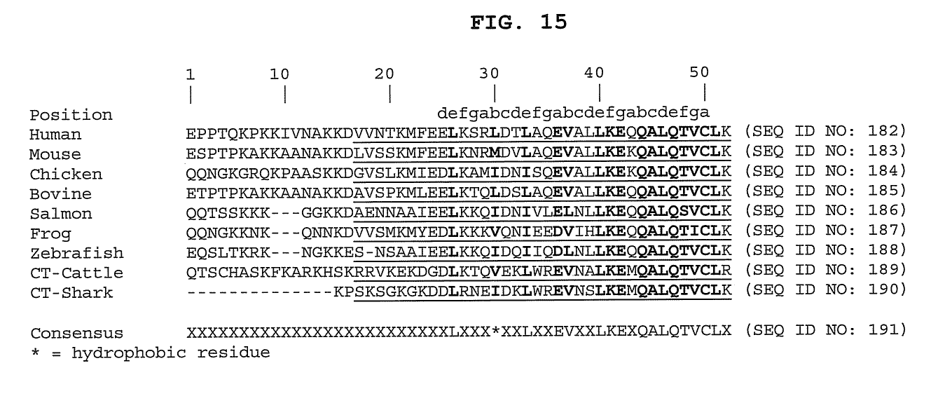

PCR can be used to generate a first fragment (fragment A, see FIG. 12) using the following strategy. Forward oligo 1Xfor (5′-GG CTG GGC CTG AAC GAC ATG NNK NNK NNK NNK NNK NNK NNK TGG GTG GAT ATG ACT GGC GCC-3′; SEQ ID NO: 202) wherein N=A, T, G or C, and K=G or T, encodes the region surrounding loop 1 of the CTLD, but replaces 15 nucleotides coding for five amino acids (AAEGT) of loop 1 with seven NNK codons. These NNK codons encoding seven random amino acids replace the wild type codons encoding the five native tetranectin amino acids. Oligo 1Xfor (SEQ ID NO: 203) can be annealed with the reverse oligo 1Xrev2 (5′-GGC GGT GAT CTC AGT TTC CCA GTT CTT GTA GGC GAT GCG GGC GCC AGT CAT ATC CAC CCA-3′; SEQ ID NO: 204). The two oligos are complementary across 21 nucleotides of their 3′ ends. Referring to FIG. 7, PCR is used to generate Fragment A (101 bp) from these two overlapping oligos. Similarly, a Fragment B (see FIG. 12) can be created by performing PCR using forward oligo BstX1 for (5′-ACT GGG AAA CTG AGA TCA CCG CCC AAC CTG ATG GCG GCG CAA CCG AGA ACT GCG CGG TCC TG-3′; SEQ ID NO: 205) and the reverse primer PstBssRevC (5′-CCC TGC AGC GCT TGT CGA ACC ACT TGC CGT TGG CGG CGC CAG ACA GGA CCG CGC AGT TCT-3′; SEQ ID NO: 206) to generate a 105 bp fragment. PCR can be performed using a high fidelity polymerase or taq blend and standard PCR thermocycling conditions. The 3′ end of fragment A is complementary to the 5′ end of fragment B. These fragments can be gel isolated and subsequently combined for overlap extension PCR using outer primers Bglfor12 and PstRev. The resulting 195 bp fragment can be gel isolated and then digested with the restriction enzymes Bgl II and Pst I, after which the final 185 bp fragment can be gel isolated and cloned into a phage display vector (such as CANTAB 5E) containing the restriction modified CTLD shown below fused to Gene III, which is similarly digested with Bgl II and Pst I for cloning.

Modification of other loops by replacement with randomized amino acids can be similarly performed as described herein. The replacement of defined amino acids within a loop with randomized amino acids is not restricted to any specific loop, nor is it restricted to the original size of the loops. Likewise, total replacement of the loop is not required, partial replacement is possible for any of the loops. In some cases retention of some of the original amino acids within the loop, such as the calcium coordinating amino acids, may be desirable. In these cases, replacement with randomized amino acids may occur for either fewer of the amino acids within the loop to retain the calcium coordinating amino acids, or additional randomized amino acids may be added to the loop to increase the overall size of the loop yet still retain these calcium coordinating amino acids. Very large peptides can be accommodated and tested by combining loop regions, such as loops 1 and 2 or loops 3 and 4, into one larger replacement loop.

The nucleic acid molecules can be obtained by ordinary methods for chemical synthesis of nucleic acids by directing the step-wise synthesis to add pre-defined combinations of pure nucleotide monomers or a mixture of any combination of nucleotide monomers at each step in the chemical synthesis of the nucleic acid fragment. In this way it is possible to generate any level of sequence degeneracy, from one unique nucleic acid sequence to the most complex mixture, which will represent a complete or incomplete representation of maximum number unique sequences of 4^{N}, where N is the number of nucleotides in the sequence.

Complex compositions comprising a plurality of nucleic acid fragments can, alternatively, be prepared by generating mixtures of nucleic acid fragments by chemical, physical or enzymatic fragmentation of high-molecular mass nucleic acid compositions such as, for example, genomic nucleic acids extracted from any organism. To render such mixtures of nucleic acid fragments useful in the generation of recombinant libraries, as described here, the crude mixtures of fragments, obtained in the initial cleavage step, would typically be size-fractionated to obtain fragments of an approximate molecular mass range which would then typically be adjoined to a suitable pair of linker nucleic acids, designed to facilitate insertion of the linker-embedded mixtures of size-restricted oligonucleotide fragments into the receiving nucleic acid vector.

Nucleic acid fragments can be inserted in specific locations into receiving nucleic acids by any common method of molecular cloning of nucleic acids, such as by appropriately designed PCR manipulations in which chemically synthesized nucleic acids are copy-edited into the receiving nucleic acid, in which case no endonuclease restriction sites are required for insertion. Alternatively, the insertion/excision of nucleic acid fragments may be facilitated by engineering appropriate combinations of endonuclease restriction sites into the target nucleic acid into which suitably designed oligonucleotide fragments may be inserted using standard methods of molecular cloning of nucleic acids.

After rounds of selection on specific targets (e.g. eukaryotic cells, virus, bacteria, specific proteins, polysaccharides, other polymers, organic compounds etc.) DNA is isolated from the specific phages, and the nucleotide sequence of the segments encoding the ligand-binding region determined, excised from the phagemid DNA and transferred to the appropriate derivative expression vector for heterologous production of the desired product. Heterologous production in a prokaryote can be used for the isolation of the desired product.

Strategy 3

In some case direct cloning of peptides with binding activity may not be enough, and further optimization and selection may be required. As an example, peptides with known binding to HSP, such as but not limited to those mentioned above, can be grafted into the CTLD of human tetranectin. In order to select for optimal presentation of these peptides for binding, one or more of the flanking amino acids can be randomized, followed by phage display selection for binding. Furthermore, peptides which alone show limited or weak binding can also be grafted into one of the loops of a CTLD library containing randomization of another additional loop, again followed by selection through phage display for increased binding and/or specificity. Additionally, for peptides identified through crystal structures where the specific interacting/binding amino acids are known, randomization of the non binding amino acids can be explored followed by selection through page display for increased binding and receptor specificity.

In various embodiments, the CTLD polypeptide sequences that bind the HSP70 activating region can have binding affinities that are about equal to the binding affinities of naturally occurring ligands for the the HSP70 activating region. In certain embodiments, the polypeptides of the invention have a binding affinity for the HSP70 activating region that is stronger than the binding affinity that a native ligand has for the same target. Such polypeptides are useful, for example, for blocking the activity of HSP70 in some cases. In other embodiments, the polypeptides of the invention have a binding affinity for the HSP70 activating region that is weaker than the binding affinity that a native ligand has for the same target. CTLD polypeptides having a weaker affinity for the HSP70 activating region than a native ligand may have an improved ability to penetrate tumors or tissues and/or may be useful in cases where the desired goal is to dampen the activity of the target rather than completely block it.

The respective binding affinity of the ligands to HSP70 can be determined and compared to the binding properties of native ligands, or a portion thereof, by ELISA, RIA, and/or BIAcore assays, as well as other assays known in the art. In certain embodiments, the receptor-selective agonists of the invention inhibit or induce a biological activity in at least one type of mammalian cell (e.g., a cancer cell), and such activity can be determined by known art methods.

In embodiments wherein the CTLD-based protein products are derived from a mammalian tetranectin, as exemplified herein with murine and human tetranectin, the structure is nearly identical with all other mammalian tetranectins. This species-conserved structure allows for straightforward swapping of polypeptide segments defining ligand-binding specificity between orthologs (e.g. murine and human tetranectin derivatives). Thus, in such embodiments, this platform provides a particular advantage over the “humanization” of murine antibody derivatives, which can involve a number of complications.

In one aspect, the invention provides a polypeptide having a multimerizing domain and comprises at least one CTLD polypeptide-binding member that binds to the HSP70 activating region. As used herein, the term “multimerizing domain” means an amino acid sequence that comprises the functionality that can associate with two or more other amino acid sequences to form trimers or other multimeric complexes. In various embodiment so of the invention, the multimerizing domain is a dimerizing domain, a trimerizing domain, a tetramerizing domain, a pentamerizing domain, etc. These domains are capable of forming polypeptide complexes of two, three, four, five or more polypeptides of the invention.

In one embodiment, the multimerized polypeptide is a trimer, for example a tetranectin trimerizing module (see US 2007/0154901). A trimeric complex including a CTLD is referred to herein as an ATRIMER™ polypeptide complex, which is a a trimeric complex of three trimerizing domains that also include CLTDs (Anaphore, Inc., San Diego, Calif.).

In accordance with the invention, a binding member may either be linked to the N- or the C-terminal amino acid residue of the multimerizing domain. Also, in certain embodiments it may be advantageous to have a binding member at both the N-terminus and the C-terminus of the multimerizing domain of the monomer, thereby providing a multimeric polypeptide complex. For example, when the multimeric peptide forms trimers with like molecules, six binding members capable of binding the HSP70 activating region can be associated with a single trimeric complex.

In another aspect of the invention, a polypeptide that specifically binds to HSP70 is contained in one or more loops in the loop region of a CTLD. In this aspect, the CTLD can be attached to any known trimerizing domain at the C-terminus of the trimerizing domain. Also, a fusion protein of the invention can include a second CTLD domain, fused at the N-terminus of the trimerizing domain. In a variation of this aspect, the fusion protein includes a polypeptide that binds to the HSP70 activating region at one of the termini of the trimerizing domain and a CTLD at the other of the termini. One, two or three such proteins can be part of a trimeric complex containing up to six specific CTLD binding members for the HSP70 activating region.

In another aspect, the invention provides a multimeric complex of three proteins, each of the proteins comprising a multimerizing domain and at least one CTLD polypeptide that binds to the HSP70 activating region. In one embodiment, the multimeric complex comprises a fusion protein having a multimerizing domain selected from a tetranectin trimerizing structural element (tetranectin trimerizing module), a mannose binding protein (MBP) trimerizing domain, a collectin neck region, and other similar moieties. The multimeric complex can be comprised of multimerizing domains that are able to associate with each other to form a multimer. Accordingly, in certain embodiments, the multimeric complex is a homomultimeric complex comprised of proteins having the same amino acid sequences. In other embodiments, the multimeric complex is a heteromultimeric complex comprised of proteins having different amino acid sequences such as, for example, different multimerizing domains, and/or different CTLD polypeptides that bind to the HSP70 activating region

In one particular embodiment, the cysteine at position 50 (C50) of SEQ ID NO: 23 can be advantageously mutagenized to serine, threonine, methionine or to any other amino acid residue in order to avoid formation of an unwanted inter-chain disulphide bridge, which can lead to unwanted multimerization. Other known variants include at least one amino acid residue selected from amino acid residue nos. 6, 21, 22, 24, 25, 27, 28, 31, 32, 35, 39, 41, and 42 (numbering according to SEQ ID NO: 23), which may be substituted by any non-helix breaking amino acid residue. These residues have been shown not to be directly involved in the intermolecular interactions that stabilize the trimeric complex between three TTSEs of native tetranectin monomers. In one aspect shown in FIG. 10, the TTSE has a repeated heptad having the formula a-b-c-d-e-f-g (N to C), wherein residues a and d (i.e., positions 26, 30, 33, 37, 40, 44, 47, and 51 may be any hydrophobic amino acid (numbering according to SEQ ID NO: 23).

In further embodiments, the TTSE trimerization domain can be modified by the incorporation of polyhistidine sequence and/or a protease cleavage site, e.g, Blood Coagulating Factor Xa or Granzyme B (see US 2005/0199251, which is incorporated herein by reference), and by including a C-terminal KG or KGS sequence. Also, to assist in purification, Proline at position 2 may be substituted with Glycine.

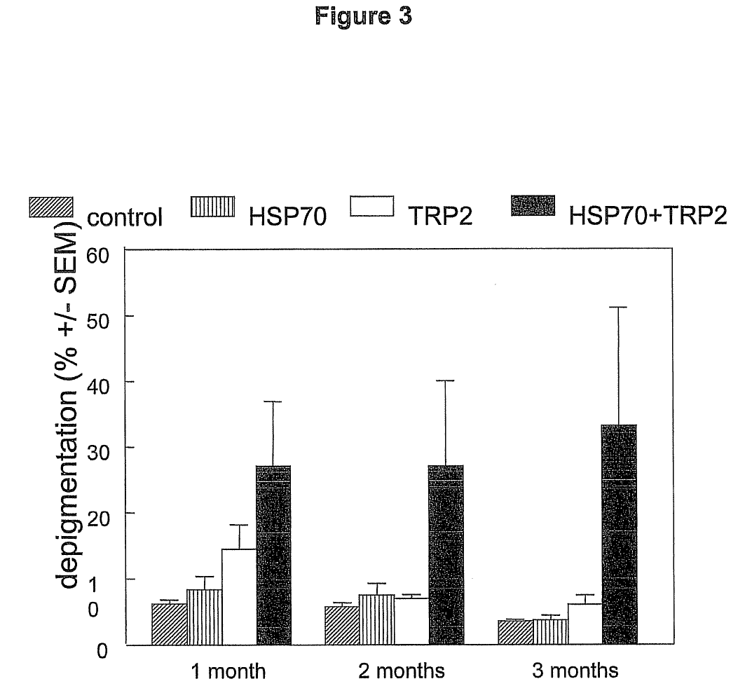

Particular non-limiting examples of TTSE truncations and variants are shown in PCT US09/60271 (FIGS. 3A-3D) and US 2010-0028995 (FIGS. 22 and 23A-C), each of which is incorporated by reference herein in its entirety. In addition, a number of trimerizing domains having substantial homology (greater than 66%) to the trimerizing domain of human tetranectin known:

TABLE 1
Trimerizing Domains

| | | |
|---|---|---|
| Equus caballus TN-like | KMFEELKSQVDSLAQEVALLKEQQALQTVCL | SEQ ID NO: 24 |
| Cat TN | KMFEELKSQVDSLAQEVALLKEQQALQTVCL | SEQ ID NO: 25 |
| Mouse TN | SKMFEELKNRMDVLAQEVALLKEKQALQTVCL | SEQ ID NO: 26 |
| Rat TN | KMFEELKNRLDVLAQEVALLKEKQALQTVCL | SEQ ID NO: 27 |
| Bovine TN | KMLEELKTQLDSLAQEVALLKEQQALQTVCL | SEQ ID NO: 28 |
| Equus caballus CTLD | DLKTQVEKLWREVNALKEMQALQTVCL | SEQ ID NO: 29 |
| like | | |
| Canis lupus CTLD | DLKTQVEKLWREVNALKEMQALQTVCL | SEQ ID NO: 30 |
| member A | | |
| Bovine CTLD member A | DLKTQVEKLWREVNALKEMQALQTVCL | SEQ ID NO: 31 |
| Macaca mulatta CTLD | DLKTQIEKLWTEVNALKEIQALQTVCL | SEQ ID NO: 32 |
| member A | | |
| Taeniopygia guttata | DDLKTQIDKLWREVNALKEIQALQTVCL | SEQ ID NO: 33 |
| CTLD member A | | |
| Ornithorhynchus | DLKTQVEKLWREVNALKEMQALQTVCL | SEQ ID NO: 34 |
| anatinus CTLD like | | |
| Rat CTLD member A | DLKSQVEKLWREVNALKEMQALQTVCL | SEQ ID NO: 35 |
| Monodelphis domestica | DLKTQVEKLWREVNALKEMQALQTVCL | SEQ ID NO: 36 |
| CTLD member A | | |
| Shark TN | DDLRNEIDKLWREVNSLKEMQALQTVCL | SEQ ID NO: 37 |
| Taeniopygia guttata | KMIEDLKAMIDNISQEVALLKEKQALQTVCL | SEQ ID NO: 38 |
| TN-like | | |
| Gallus gallus TN | KMIEDLKAMIDNISQEVALLKEKQALQTVCL | SEQ ID NO: 39 |
| Danio rerio CTLD | DDMKTQIDKLWQEVNSLKEMQALQTVCL | SEQ ID NO: 40 |
| member A | | |
| Gallus gallus, CTLD | DDLKTQIDKLWREVNALKEMQALQSVCL | SEQ ID NO: 41 |
| member A | | |
| Mouse CTLD member A | DDLKSQVEKLWREVNALKEMQALQTVCL | SEQ ID NO: 42 |
| Gallus gallus CTLD | DDLKTQIDKLWREVNALKEMQALQSVCL | SEQ ID NO: 43 |
| member A | | |
| Tetraodon | DDVRSQIEKLWQEVNSLKEMQALQTVCL | SEQ ID NO: 44 |
| nigroviridis, unkown | | |
| Xenopus laevis | DLKTQIDKLWREINSLKEMQALQTVCL | SEQ ID NO: 45 |
| MGC85438 | | |
| Tetraodon | EELRRQVSDLAQELNILKEQQALHTVCL | SEQ ID NO: 46 |
| nigroviridis, unkown | | |
| Xenopus laevis,unkown | KMYEELKQKVQNIELEVIHLKEQQALQTICL | SEQ ID NO: 47 |
| Xenopus tropicalis TN | KMYEDLKKKVQNIEEDVIHLKEQQALQTICL | SEQ ID NO: 48 |
| Salmo salar TN | EELKKQIDNIVLELNLLKEQQALQSVCL | SEQ ID NO: 49 |
| Danio rerio TN | EELKKQIDQIIQDLNLLKEQQALQTVCL | SEQ ID NO: 50 |
| Tetraodon | EQMQKQINDIVQELNLLKEQQALQAVCL | SEQ ID NO: 51 |
| nigroviridis, unknown | | |
| Tetraodon | EQMQKQINDIVQELNLLKEQQALQAVCL | SEQ ID NO: 52 |
| nigroviridis, unkown | | |

Other human polypeptides that are known to trimerize include those found in Table 2.

TABLE 2
Trimerizing Polypeptides

| | | |
|---|---|---|
| hTRAF 3 | NTGLLESQLSRHDQMLSVHDIRLADMD | SEQ ID NO: 53 |
| | LRFQVLETASYNGVLIWKIRDYKRRKQ | |
| | EAVM | |
| hMBP | AASERKALQTEMARIKKWLTF | SEQ ID NO: 54 |
| hSPC300 | FDMSCRSRLATLNEKLTALERRIEYIE | SEQ ID NO: 55 |
| | ARVTKGETLT | |
| hNEMO | ADIYKADFQAERQAREKLAEKKELLQE | SEQ ID NO: 56 |
| | QLEQLQREYSKLKASCQESARI | |
| hcubilin | LTGSAQNIEFRTGSLGKIKLNDEDLSE | SEQ ID NO: 57 |
| | CLHQIQKNKEDIIELKGSAIGLPIYQL | |
| | NSKLVDLERKFQGLQQT | |
| hThrombos | LRGLRTIVTTLQDSIRKVTEENKELAN | SEQ ID NO: 58 |
| pondins | E | |

Another example of a trimmerizmg domain is U.S. Pat. No. 6,190,886 (incorporated by reference herein in its entirety), which describes polypeptides comprising a collectin neck region. Trimers can then be made under appropriate conditions with three polypeptides comprising the collectin neck region amino acid sequence. A number of collectins are identified, including:

Collectin neck region of human SP-D:

| | |
|---|---|
| VASLRQQVEALQGQVQHLQAAFSQYKK | [SEQ ID NO: 59] |

Collectin neck region of bovine SP-D:

| | |
|---|---|
| VNALRQRVGILEGQLQRLQNAFSQYKK | [SEQ ID NO: 60] |

Collectin neck region of rat SP-D:

| | |
|---|---|
| SAALRQQMEALNGKLQRLEAAFSRYKK | [SEQ ID NO: 61] |

Collectin neck region of bovine conglutinin:

| | |
|---|---|
| VNALKQRVTILDGHLRRFQNAFSQYKK | [SEQ ID NO: 62] |

Collectin neck region of bovine collectin:

| | |
|---|---|
| VDTLRQRMRNLEGEVQRLQNIVTQYRK | [SEQ ID NO: 63] |

Neck region of human SP-D:

| | |
|---|---|
| GSPGLKGDKGIPGDKGAKGESGLPDVASLRQQVEALQGQVQHLQAAFSQYKKVELFPGGIPHRD | [SEQ ID NO: 64] |

The invention also provides for a general and simple procedure for reliable conversion of an initially selected protein derivative into a final protein product, which without further reformatting may be produced in bacteria (e.g. Escherichia coli) both in small and in large scale (International Patent Application Publication No. WO 94/18227 A2). In certain embodiments, several identical or non-identical binding sites can be included in the same functional protein unit by simple and general means, enabling the exploitation even of weak affinities by means of avidity in the interaction, or the construction of bi- or hetero-functional molecular assemblies (International Patent Application Publication No. WO 98/56906, which is incorporated by reference in its entirety). In certain embodiments, binding can be modulated by the addition or removal of divalent metal ions (e.g. calcium ions) in combinational libraries with one or more preserved metal binding site(s) in the CTLDs. Alternatively, binding can be modulated by altering the pH.

Strategies for Identifying and Isolating CTLD polypeptides that bind to the HSP70 activating region.

In one aspect, the invention provides a method for identifying and isolating a polypeptide having specific binding activity to the HSP70 activating region, wherein the method comprises (a) providing a combinatorial polypeptide library of the invention; (b) contacting the polypeptides of the combinatorial polypeptide library with a polypeptide having the HSP70 activating region under conditions that allow for binding between a polypeptide and the HSP70 activating region; and (c) isolating a polypeptide that binds to the HSP70 activating region. In another aspect, the invention provides a method for identifying and isolating a polypeptide having specific binding activity to the HSP70 activating region, wherein the method further comprises a library of nucleic acid molecules encoding polypeptides of the combinatorial polypeptide library, wherein the library of nucleic acids is expressed in a display system. In one embodiment, the display system comprises an observable phenotype that represents at least one property of the displayed expression products and the corresponding genotypes.

A specific binding member for the HSP70 activating region can be obtained from a random library of polypeptides by selection of members of the library that specifically bind to the HSP70 activating region. As discussed herein, a number of systems for displaying phenotypes with putative ligand binding sites are known. These include: phage display (e.g. the filamentous phage fd [Dunn (1996), Griffiths and Duncan (1998), Marks et al. (1992)], phage lambda [Mikawa et al. (1996)]), display on eukaryotic virus (e.g. baculovirus [Ernst et al. (2000)]), cell display (e.g. display on bacterial cells [Benhar et al. (2000)], yeast cells [Boder and Wittrup (1997)], and mammalian cells [Whitehorn et al. (1995)], ribosome linked display [Schaffitzel et al. (1999)], and plasmid linked display [Gates et al. (1996)].

To select for polypeptides with binding activity to the HSP70 activating region, libraries can be constructed and initially screened for binding to the HSP70 activating region as monomeric elements, either as single monomeric CTLD domains or individual peptides displayed on the surface of phage. Libraries can be constructed by randomizing the amino acids in one or more of the five different loops (or outside the loops) within the CTLD scaffold displayed on the surface of phage. Binding to the HSP70 activating region can be selected for by phage display panning.

Several strategies can be employed in the construction of phage display libraries. One strategy is to construct and/or use random peptide phage display libraries. Random linear peptides and/or random peptides constructed as disulfide constrained loops can be individually displayed on the surface of phage particles and selected for binding to the HSP70 activating region through phage display “panning”. After obtaining peptide clones with the desired binding activity, these peptides can be grafted on to the trimerization domain of human tetranectin or into loops of the CTLD domain followed by grafting on the trimerization domain and screened for agonist activity.

Another strategy for construction of phage display libraries and trimerization domain constructs include obtaining CTLD derived binders. Libraries can be constructed by randomizing the amino acids in one or more of the five different loops within the CTLD scaffold (i.e., of human tetranectin) displayed on the surface of phage. Binding to the HSP70 activating region can be selected for through phage display panning. After obtaining CTLD clones with peptide loops demonstrating the desired binding activity, the CTLD clones can then be grafted on to the trimerization domain of human tetranectin and screened for agonist activity.

Another strategy includes using peptide sequences with known binding capabilities to the target of interest and first improving their binding by creating new libraries with randomized amino acids flanking the peptide or/and randomized selected internal amino acids within the peptide, followed by selection for improved binding through phage display. After obtaining binders with improved affinity, the binders of these peptides can be fused to other functional protein domains such as, for example, the trimerization domain of human tetranectin (discussed herein and discussed in detail in PCT/US09/60271 and US. 2010/0028995, which are incorporated herein by reference in their entirety), and evaluated for desired activity. In this method, initial libraries can be constructed as either free peptides displayed on the surface of phage particles, as in the first strategy, or as constrained loops within the CTLD scaffold as in the second strategy discussed above. These display strategies are described in detail in PCT/US09/60271, which is incorporated by reference herein in its entirety.

Strategy 4:

Once a number of peptides with binding activity to HSP70 have been identified, these peptides can be cloned directly on to either the N or C terminal end trimerization domain as free linear peptides or as disulfide constrained loops using cysteines. Single chain antibodies or domain antibodies capable of binding the HSP can also be cloned on to either end of the trimerization domain. Additionally peptides with known binding properties can be cloned directly into any one of the loop regions of the TN CTLD. Peptides selected for as disulfide constrained loops or as complementary determining regions of antibodies might be quite amenable to relocation into the loop regions of the CTLD of human tetranectin. For all of these constructs, binding as a monomer, as well as binding as a trimer, when fused with the trimerization domain can then be tested.

Pharmaceutical Compositions

In yet another aspect, the invention relates to a pharmaceutical composition comprising a therapeutically effective amount of the fusion protein of the invention along with a pharmaceutically acceptable carrier or excipient. As used herein, “pharmaceutically acceptable carrier” or “pharmaceutically acceptable excipient” includes any and all solvents, dispersion media, coating, antibacterial and antifungal agents, isotonic and absorption delaying agents, and the like that are physiologically compatible. Examples of pharmaceutically acceptable carriers or excipients include one or more of water, saline, phosphate buffered saline, dextrose, glycerol, ethanol and the like as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the of the antibody or antibody portion also may be included. Optionally, disintegrating agents can be included, such as cross-linked polyvinyl pyrrolidone, agar, alginic acid or a salt thereof, such as sodium alginate and the like. In addition to the excipients, the pharmaceutical composition can include one or more of the following, carrier proteins such as serum albumin, buffers, binding agents, sweeteners and other flavoring agents; coloring agents and polyethylene glycol.

The compositions can be in a variety of forms including, for example, liquid, semi-solid and solid dosage forms, such as liquid solutions (e.g. injectable and infusible solutions), dispersions or suspensions, tablets, pills, powders, liposomes and suppositories. The preferred form will depend on the intended route of administration and therapeutic application. In an embodiment the peptide, complex or composition is administered in a topical cream or ointment. In an embodiment the compositions are in the form of injectable or infusible solutions, such as compositions similar to those used for passive immunization of humans with antibodies. In an embodiment the mode of administration is parenteral (e.g., intravenous, subcutaneous, intraperitoneal, intramuscular). In an embodiment, the fusion protein (or trimeric complex) is administered by intravenous infusion or injection. In another embodiment, the fusion protein or trimeric complex is administered by intramuscular or subcutaneous injection.

Other suitable routes of administration for the pharmaceutical composition include, but are not limited to, rectal, transdeunal, vaginal, transmucosal or intestinal administration.

Therapeutic compositions are typically sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e. fusion protein or trimeric complex) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An article of manufacture such as a kit containing HSP70 polypeptide binders and therapeutic agents useful in the treatment of the disorders described herein comprises at least a container and a label. Suitable containers include, for example, bottles, vials, syringes, and test tubes. The containers may be formed from a variety of materials such as glass or plastic. The label on, or associated with, the container indicates that the formulation is used for treating the condition of choice. The article of manufacture may further comprise a container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a container with another active agent as described above.

Typically, an appropriate amount of a pharmaceutically-acceptable salt is used in the formulation to render the formulation isotonic. Examples of pharmaceutically-acceptable carriers include saline, Ringer's solution and dextrose solution. The pH of the formulation is preferably from about 6 to about 9, and more preferably from about 7 to about 7.5. It will be apparent to those persons skilled in the art that certain carriers may be more preferable depending upon, for instance, the route of administration and concentrations of the HSP polypeptide binders.

Therapeutic compositions can be prepared by mixing the desired molecules having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl amrnonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Formulations to be used for in vivo administration should be sterile. This is accomplished by filtration through sterile filtration membranes, prior to or following lyophilization and reconstitution. The formulation may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic formulations generally are placed into a container having a sterile access port, for example, an intravenous solution bag or vial having a stopper pierceable by a hypodermic injection needle. The formulations are preferably administered as repeated topical, intravenous (i.v.), subcutaneous (s.c.), intramuscular (i.m.) injections or infusions, or as aerosol formulations suitable for intranasal or intrapulmonary delivery.

The molecules disclosed herein can also be administered in the form of sustained-release preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988).

Supplementary active compounds also can be incorporated into the compositions. In certain embodiments, a fusion protein or trimeric complex of the invention is co-formulated with and/or co-administered with one or more additional therapeutic agents. For example, a fusion protein or trimeric complex of the invention may be co-formulated and/or co-administerd with one or more antibodies that bind other targets (e.g., antibodies that bind other cytokines or that bind cell surface molecules) or one or more cytokines.

As used herein, the term “therapeutically effective amount” means an amount of fusion protein or trimeric complex that produces the effects for which it is administered. The exact dose will be ascertainable by one skilled in the art. As known in the art, adjustments based on age, body weight, sex, diet, time of administration, drug interaction and severity of condition may be necessary and will be ascertainable with routine experimentation by those skilled in the art. A therapeutically effective amount is also one in which the therapeutically beneficial effects outweigh any toxic or detrimental effects of the fusion protein or trimeric complex. A “prophylactically effective amount” refers to an amount effective, at dosages and for periods of time necessary to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be tested; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved and (b) the limitations inherent in the art of compounding such an active compound for the treatment of sensitivity in individuals.

Methods of Treatment

Another aspect the invention relates to a method of preventing the HSP70 mediated activation of DCs. The method includes contacting soluble HSP70 with a binding member for the HSP70 dendritic cell activating region of the invention that includes a trimerizing domain and at least one polypeptide that binds to the HSP70 activating region. In one embodiment of this aspect, the method comprises contacting tissue containing cells expressing HSP70 with a trimeric complex of the invention.

The HSP polypeptide binders can be administered in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Optionally, administration may be performed through mini-pump infusion using various commercially available devices.

The invention is also directed to a method of treating melanoma that includes administering the polypeptides, fusion protein or complexes of the invention to a patient suffering from melanoma.

Effective dosages and schedules for administering the HSP polypeptide and polypeptide binders of the invention may be determined empirically, and making such determinations is within the skill in the art. Single or multiple dosages may be employed. When in vivo administration of the HSP polypeptide and polypeptide binders is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature. See, for example, U.S. Pat. Nos. 4,657,760; 5,206,344; or 5,225,212. Those skilled in the art will understand that the dosage that must be administered will vary depending on, for example, the animal that will receive the polypeptide, the route of administration, and other drugs or therapies being administered to the mammal. Interspecies scaling of dosages can be performed in a manner known in the art; e.g, as disclosed in Mordenti et al., Pharmaceut. Res., 8:1351 (1991).

With respect to therapeutic uses, the CTLD-based protein products are identical to the corresponding natural CTLD protein already present in the body, and are therefore expected to elicit minimal immunological response in the patient. Single CTLDs are about half the mass of an antibody and may in some applications be advantageous as it may provide better tissue penetration and distribution, as well as a shorter half-life in circulation. Multivalent formats of CTLD proteins may provide increased binding capacity and avidity and longer circulation half-life.

Production of Fusion Proteins

The fusion protein of the invention can be expressed in any suitable standard protein expression system by culturing a host transformed with a vector encoding the fusion protein under such conditions that the fusion protein is expressed. Preferably, the expression system is a system from which the desired protein may readily be isolated and refolded in vitro. As a general matter, prokaryotic expression systems are preferred since high yields of protein can be obtained and efficient purification and refolding strategies are available. Thus, selection of appropriate expression systems (including vectors and cell types) is within the knowledge of one skilled in the art. Similarly, once the primary amino acid sequence for the fusion protein of the present invention is chosen, one of ordinary skill in the art can easily design appropriate recombinant DNA constructs which will encode the desired amino acid sequence, taking into consideration such factors as codon biases in the chosen host, the need for secretion signal sequences in the host, the introduction of proteinase cleavage sites within the signal sequence, and the like.

In one embodiment the isolated polynucleotide encodes an HSP polypeptide or a polypeptide that binds an HSP70 activating region. In an embodiment the isolated polynucleotide encodes a first polypeptide that binds an HSP70 polypeptide, a second polypeptide that binds an HSP70 polypeptide, and a trimerizing domain. In certain embodiments, the polypeptide that binds an HSP70 polypeptide (or the first polypeptide and the second polypeptide) and the trimerizing domain are encoded in a single contiguous polynucleotide sequence (a genetic fusion). In other embodiments, polypeptide that binds an HSP70 polypeptide (or the first polypeptide and the second polypeptide) and the trimerizing domain are encoded by non-contiguous polynucleotide sequences. Accordingly, in some embodiments at least one polypeptide that binds an HSP70 polypeptide (or the first polypeptide and second polypeptide that specifically bind an HSP70 polypeptide) and the trimerizing domain are expressed, isolated, and purified as separate polypeptides and fused together to form the fusion protein of the invention.

Standard techniques may be used for recombinant DNA molecule, protein, and fusion protein production, as well as for tissue culture and cell transformation. See, e.g., Sambrook, et al. (below) or Current Protocols in Molecular Biology (Ausubel et al., eds., Green Publishers Inc. and Wiley and Sons 1994). Purification techniques are typically performed according to the manufacturer's specifications or as commonly accomplished in the art using conventional procedures such as those set forth in Sambrook et al. (Molecular Cloning: A Laboratory Manual. Cold Spring Harbor Laboratory Press, Cold Spring Harbor, N.Y. (1989), or as described herein. Unless specific definitions are provided, the nomenclature utilized in connection with the laboratory procedures, and techniques relating to molecular biology, biochemistry, analytical chemistry, and pharmaceutical/formulation chemistry described herein are those well known and commonly used in the art. Standard techniques can be used for biochemical syntheses, biochemical analyses, pharmaceutical preparation, formulation, and delivery, and treatment of patients.

These recombinant DNA constructs may be inserted in-frame into any of a number of expression vectors appropriate to the chosen host. In certain embodiments, the expression vector comprises a strong promoter that controls expression of the recombinant fusion protein constructs. When recombinant expression strategies are used to generate the fusion protein of the invention, the resulting fusion protein can be isolated and purified using suitable standard procedures well known in the art, and optionally subjected to further processing such as e.g. lyophilization.

It will be appreciated that a flexible molecular linker optionally may be interposed between, and covalently join, the specific binding member and the trimerizing domain. In certain embodiments, the linker is a polypeptide sequence of about 1-20 amino acid residues. The linker may be less than 10 amino acids, most preferably, 5, 4, 3, 2, or 1. It may be in certain cases that 9, 8, 7 or 6 amino acids are suitable. In useful embodiments the linker is essentially non-immunogenic, not prone to proteolytic cleavage and does not comprise amino acid residues which are known to interact with other residues (e.g. cysteine residues).

The description below also relates to methods of producing fusion proteins and trimeric complexes that are covalently attached (hereinafter “conjugated”) to one or more chemical groups. Chemical groups suitable for use in such conjugates are preferably not significantly toxic or immunogenic. The chemical group is optionally selected to produce a conjugate that can be stored and used under conditions suitable for storage. A variety of exemplary chemical groups that can be conjugated to polypeptides are known in the art and include for example carbohydrates, such as those carbohydrates that occur naturally on glycoproteins, polyglutamate, and non-proteinaceous polymers, such as polyols (see, e.g., U.S. Pat. No. 6,245,901).

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, poly(propylene glycol) and polyethylene-polypropylene glycol copolymers, can be employed using the techniques for conjugation described herein for PEG. The polyols of the invention include those well known in the art and those publicly available, such as from commercially available sources.

A polyol, for example, can be conjugated to fusion proteins of the invention at one or more amino acid residues, including lysine residues, as is disclosed in WO 93/00109, supra. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, poly(propylene glycol) and polyethylene-polypropylene glycol copolymers, can be employed using the techniques for conjugation described herein for PEG.

The average molecular weight of the PEG employed in the pegylation of the Apo-2L can vary, and typically may range from about 500 to about 30,000 daltons (D). Preferably, the average molecular weight of the PEG is from about 1,000 to about 25,000 D, and more preferably from about 1,000 to about 5,000 D. In one embodiment, pegylation is carried out with PEG having an average molecular weight of about 1,000 D. Optionally, the PEG homopolymer is unsubstituted, but it may also be substituted at one end with an alkyl group. Preferably, the alkyl group is a C1-C4 alkyl group, and most preferably a methyl group. PEG preparations are commercially available, and typically, those PEG preparations suitable for use in the present invention are nonhomogeneous preparations sold according to average molecular weight. For example, commercially available PEG(5000) preparations typically contain molecules that vary slightly in molecular weight, usually ±500 D. The fusion protein of the invention can be further modified using techniques known in the art, such as, conjugated to a small molecule compounds (e.g., a chemotherapeutic); conjugated to a signal molecule (e.g., a fluorophore); conjugated to a molecule of a specific binding pair (e.g,. biotin/streptavidin, antibody/antigen); or stabilized by glycosylation, PEGylation, or further fusions to a stabilizing domain (e.g., Fc domains).

A variety of methods for pegylating proteins are known in the art. Specific methods of producing proteins conjugated to PEG include the methods described in U.S. Pat. Nos. 4,179,337, 4,935,465 and 5,849,535. Typically the protein is covalently bonded via one or more of the amino acid residues of the protein to a terminal reactive group on the polymer, depending mainly on the reaction conditions, the molecular weight of the polymer, etc. The polymer with the reactive groups) is designated herein as activated polymer. The reactive group selectively reacts with free amino or other reactive groups on the protein. The PEG polymer can be coupled to the amino or other reactive group on the protein in either a random or a site specific manner. It will be understood, however, that the type and amount of the reactive group chosen, as well as the type of polymer employed, to obtain optimum results, will depend on the particular protein or protein variant employed to avoid having the reactive group react with too many particularly active groups on the protein. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

It should be noted that the section headings are used herein for organizational purposes only, and are not to be construed as in any way limiting the subject matter described. All references cited herein are incorporated by reference in their entirety for all purposes.

The following are provided for exemplification purposes only and are not intended to limit the scope of the invention described in broad terms above. All references cited in this disclosure are incorporated herein by reference.

### Examples

### Example 1

Mutations were introduced into the human HSP70 expression vector to better understand the DC activating region in human HSP70 and to identify the smallest possible region that is involved in activating DCs. The amino acid sequence of human HSP70 is shown below [SEQ ID NO:16].

| | MAKAAAIGID LGTTYSCVGV FQHGKVEIIA NDQGNRTTPS YVAFTDTERL IGDAAKNQVA |
|---|---|
| 61 | LNPQNTVFDA KRLIGRKFGD PVVQSDMKHW PFQVINDGDK PKVQVSYKGE TKAFYPEEIS |
| 121 | SMVLTKMKEI AEAYLGYPVT NAVITVPAYF NDSQRQATKD AGVIAGLNVL RIINEPTAAA |
| 181 | IAYGLDRTGK GERNVLIFDL GGGTFDVSIL TIDDGIFEVK ATAGDTHLGG EDFDNRLVNH |
| 241 | FVEEFKRKHK KDISQNKRAV RRLRTACERA KRTLSSSTQA SLEIDSLFEG IDFYTSITRA |
| 301 | RFEELCSDLF RSTLEPVEKA LRDAKLDKAQ IHDLVLVGGS TRIPKVQKLL QDFFNGRDLN |
| 361 | KSINPDEAVA YGAAVQAAIL MGDKSENVQD LLLLDVAPLS LGLETAGGVM TALIKRNSTI |
| 421 | PTKQTQIFTT YSDNQPGVLI QVYEGERAMT KDNNLLGRFE LSGIPPAPRG VPQIEVTFDI |
| 481 | DANGILNVTA TDKSTGKANK ITITNDKGRL SKEEIERMVQ EAEKYKAEDE VQRERVSAKN |
| 541 | ALESYAFNMK SAVEDEGLKG KISEADKKKV LDKCQEVISW LDANTLAEKD EFEHKRKELE |
| 601 | QVCNPIISGL YQGAGGPGPG GFGAQGPKGG SGSGPTIEEV D |

A 13-mer (in bold) of HSP-70 was chosen for further investigation of its significance for depigmentation in vitiligo. Four mutants were generated and the vectors containing these sequences were tested in the Vitiligo mouse model as described in Denman et al., Society for Investigative Dermatology, 128; 2041-2048, March 2008, hereby incorporated by reference. The model utilizes human TRP-2 DNA to direct translation of proteins that provide melanocyte-related antigenic peptides which are recognized by dendritic cells, thereby inducing a T-cell mediated immune response. Mutations were introduced into the HSP70 encoding plasmid by site-directed mutagenesis. Table 3 below shows the results of this site directed mutagenesis. Mutations were introduced to alter 1 or 2 of the amino acids within the 13-mer. Modified amino acids are shown in bold.

TABLE 3

| | | |
|---|---|---|
| Wild type | QPGVLIQVYEGER | SEQ ID NO: 1 |
| Mutant 5 | QPGKLAQVYEGER | SEQ ID NO: 8 |
| Mutant 6 | QPGVLIQAVEGER | SEQ ID NO: 9 |
| Mutant 8 | APGVLIQVYEGER | SEQ ID NO: 10 |
| Mutant 10 | QPGVLIQVYEGVA | SEQ ID NO: 11 |

Cloning and sequencing of hTRP-2 and hHSP70 and hHSP70 mutants can be accomplished as follows. For hTRP-2 expression cloning, RNA was isolated from M14 human melanoma cells. TRP-2 transcripts can be amplified in the presence of the following primers: 5′-CACCATGAGCCCCC TTTGGTGGGGGTTTC-3′ (forward) [SEQ ID NO: 12] and 5′-CTAGGCTTCTTCTGTG TATCTCTTG-3′ (reverse) [SEQ ID NO: 13]. The CACC sequence in the upstream primer allowed for directional TOPO cloning of the PCR product into pcDNA3.1D/V5-His-TOPO (Invitrogen, Carlsbad, Calif.). Human HSP70i can be amplified from human primary keratinocyte RNA in the presence of primers 5′-ATGGCCGCGGCGATCG-3′ (forward) [SEQ ID NO: 14]and 5′-CTAATCTACCTCAATGGTG-3′ (reverse) [SEQ ID NO: 15]. HSP70-encoding genes were cloned into pcDNA3.1/CT-GFP-TOPO (Invitrogen).

Reverse transcription PCR conditions for all amplifications can be accomplished as follows: 5 mg RNA can be combined with first strand reverse transcription buffer in presence of 1 mM each of dNTPs, 10 mM DTT (dithiothreitol), 3.3 mM MgCl₂, 25 ng/ml oligodT primer and 200 U Supercript II reverse transcriptase at 42° C., terminating the reaction by heating to 70° C. Ten percent of the reverse transcription reaction may be PCR amplified; PCR buffer: 2 mM MgCl₂, 400 mM each of dNTPs, 0.8 mg/ml primers and 5 U Taq polymerase. In the case of hTRP-2, Taq polymerase was replaced by 2.5 U AccuPrime enzyme (Invitrogen) and additives can be replaced by 1# AccuPrime mix (Invitrogen). PCR reactions can be run for 40 cycles at 95° C. for 30 seconds, 58° C. for 30 seconds, and 72° C. for 100 seconds, followed by 10 minutes at 72 1 C. PCR products can be cloned into the appropriate vectors according to the manufacturer's instructions.

Bacterial colonies from each cloning procedure can subjected to restriction analysis, and a clone containing the gene in the correct orientation may be used for a MegaPrep endotoxin-free isolation procedure (Qiagen, Valencia, Calif.) and verified by sequencing. Successful expression of all proteins encoded by eukaryotic expression vectors included in vaccines, including hHSP70, mHSP70, and TRP-2, can be confirmed by western blotting of total protein from transfected COS cells, followed by indirect alkaline phosphatase immunostaining.

### Example 2

Single or double substituted peptide sequences were introduced within the 13-mer and expression of native and mutant proteins was confirmed by Western blotting of transfected COS cells. Mutants that did not result in expression of protein are not shown.

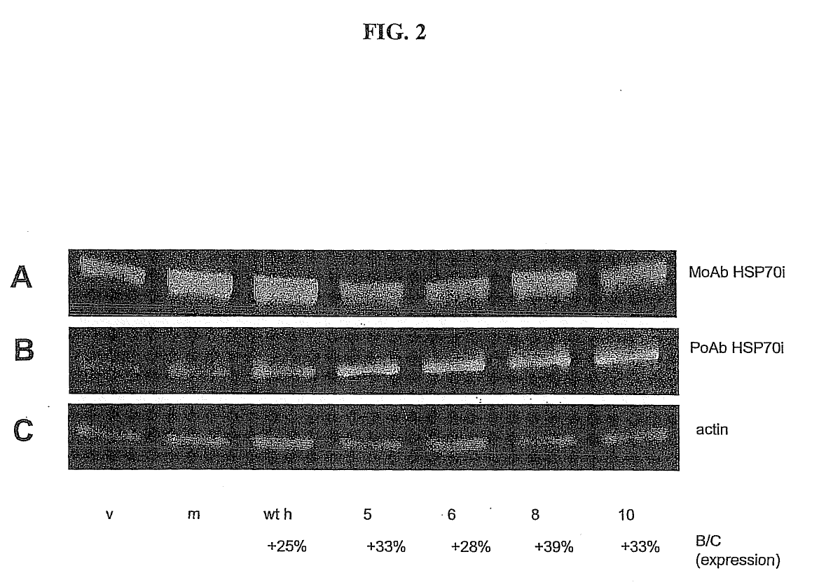

FIG. 2 shows expression of inducible HSP70 (HSP70i) by COS cells 48 h after transformation. COS cells were transfected in presence of lipofectamine for 48 hrs before protein harvesting. Blots were probed with antibodies to HSP70 (both SPA-810 (monoclonal) and SPA-811 (polyclonal)). Recognition of mutants 5 and 6 by MoAb is reduced as compared to recognition by polyclonal antibodies (PoAb). Antibodies were purchased from Assay Designs Inc., (Ann Arbor, Mich.).

### Example 3

Ten C57BL/6 mice/group were vaccinated weekly with 4.8 μg of total DNA for four weeks. Plasmid DNA used included combinations of TRP2 (used to direct immunogenic response to melanocytes) and wild type or mutant HSP70 expression vectors, as well as empty vector control group. Mice were vaccinated by gene gun as described in Overwijk, et al., PNAS, 96:2982-7, 1999. To prepare “bullets” for use in the gene gun, endotoxin-free plasmid DNA in desired combinations was precipitated onto spermidine-coated gold beads (Fluka Biochemika, Buchs, Switzerland and Sigma-Aldrich) in the presence of 200 mM CaCl2 (Sigma, St Louis, Mo.) and 10 volumes of ethanol (Sigma). Washed beads were precipitated onto silicone tubing (Bio-Rad) in a BioRad Tubing Prep Station (Bio-Rad). Bullets were used within 10 days of preparation. Two strains of mice (C57BL/6J from Jackson Labs, Bar Harbor) by gene gun vaccination using the Helios Gene Gun System (Bio-Rad). Gold particles coated with DNA of interest are released from silicon tubing cartridges under helium pressure at maximum 300 p.s.i. (pound per square inch), which allows for DNA to directly enter the skin and nestle inside relevant cell types such as DC, where the DNA can be expressed before and after migration to draining lymph nodes to induce an immune response to antigens encoded by the vaccine.

The assays utilized a group size of 10 mice per experimental condition. The mice are anaesthetised and their hair was removed with NAIR® cream prior to vaccination. Depigmentation was measured from images on a flat-bed scanner weekly after the pelage returned. Depigmentation was estimated by scanning the anaesthetized mice using a flatbed scanner on both sides once weekly and quantifying grayscale using PHOTOSHOP® software. The mice were monitored for nine weeks, including one week of acclimatization, an additional three weeks to vaccinate and eight weeks of follow-up after the pelage re-grew. The mice were entered into experiments at the age of 6-10 weeks.

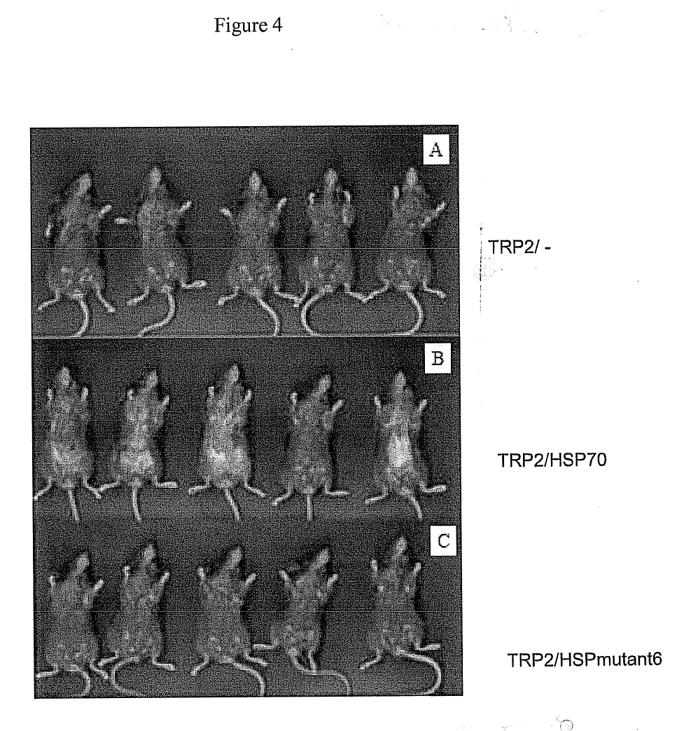

FIGS. 3 and 4 show the results of the experiments. FIG. 4A shows depigmentation of mice six weeks after the final gene gun vaccination. FIG. 4B shows mice that have been vaccinated with control plasmid only. After the pelage returned, no depigmentation was observed. FIG. 4B shows significant depigmentation in mice that were vaccinated with a combination of equal amounts of TRP2 and human HSP70 encoding plasmids. As shown in FIG. 4C, mice did not display depigmentation after vaccination with a combination of equal amounts of TRP2 and human HSP70 mutant 6 encoding plasmids. These results show the variation in penetrance of depigmentation among equally treated mice.

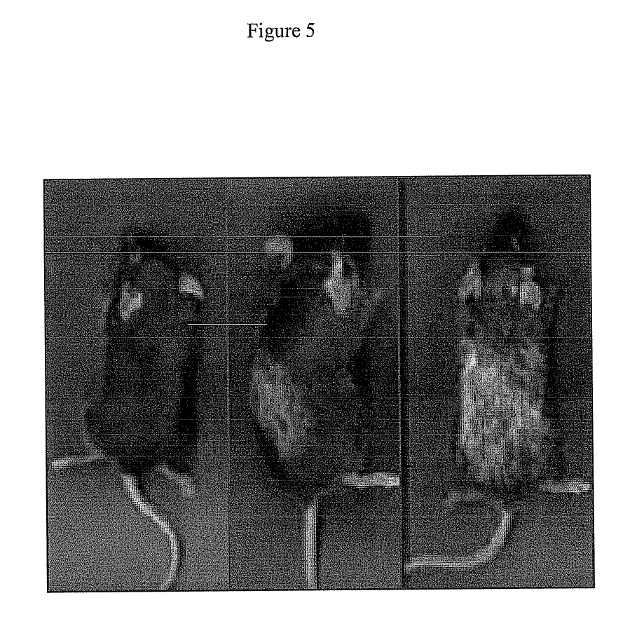

FIG. 5 shows that ventral gene gun vaccination induced depigmentation progressing to the backs of the mice. Dorsal images representing non-vaccinated areas of representative mice treated with control vector (FIG. 5, left) versus (FIG. 5, middle) a combination of TRP-2 and human HSP70 mutant 10 or (FIG. 6, right) or TRP-2 plus mouse HSP70. The progressive nature of their depigmentation is similar to that observed in human vitiligo. Dorsal depigmentation was also observed in mice treated with HSP70 mutant 10.

Therefore, it can be determined that the amino acid sequence QPGVLIQVYEG [SEQ ID NO: 1] is responsible for DC activation and immune activation thereby. FIG. 1 shows that the peptide of the invention mediates the process of autoimmune depigmentation. Comparison of the activity of wildtype peptide to mutants 5, 6, 8, and 10 indicates that only mutant 10 accelerates depigmentation to level similar to that of wildtype peptide.

In the following examples, a number of vectors are described (e.g, pANA vectors) These vectors derived from vectors that have been previously described [see US 2007/0275393] or the sequences thereof are provided in U.S. patent application Ser. No. 12/703,752 (incorporated by reference in its entirety).

The pPhCPAB phage display vector (see U.S. Ser. No. 12/703,752) is derived from pCANTAB (Pharmacia). This vector has the gill signal peptide coding region fused with a linker to the hTN sequence encoding ALQT (etc.) and contains a portion of the human tetranectin CTLD fused to the M13 gene III protein. The CTLD region is modified to include BglII and PstI restriction enzyme sites flanking Loops 1-4, and the 1-4 region is altered to include stop codons, such that no functional gene III protein could be produced from the vector without ligation of an in-frame insert. pANA27 is derived from pPhCPAB by replacing the BamHI to ClaI regions to replace the amber suppressible stop codon with a glutamine codon and truncates the amino terminal region of gene III.

The C-terminal end of the CTLD region is fused via a linker to the remaining gIII coding region. Within the CTLD region, nucleotide mutations are generated that did not alter the coding sequence but generated restriction sites suitable for cloning PCR fragments containing altered loop regions. A portion of the loop region is removed between these restriction sites so that all library phage could only express recombinants and not wild-type tetranectin. The murine TN CTLD phage display vectors are similarly designed. Another embodiment of these vectors is pANA27 in which the gene III C-terminal region is truncated and the suppressible stop codon at the end of the hTN coding sequence has been altered to encode glutamine. The murine vector pANA28 is constructed in a similar fashion.

The sequences of the primers identified by name in the Examples are provided in Table 4.

TABLE 4
Primer sequences used in the generation of phage displayed
C-type lectin domain libraries.

| | | SEQ ID |
|---|---|---|
| Name | Sequence | NO |
| 1Xfor | GGCTGGGCCT GAACGACATG NNKNNKNNKN NKNNKNNKNN KTGGGTGGAT | 87 |
| | ATGACTGGCG CC | |
| 1Xrev | GGCGGTGATC TCAGTTTCCC AGTTCTTGTA GGCGATMNNG GCGCCAGTCA | 88 |
| | TATCCACCCA | |
| BstX1for | ACTGGGAAAC TGAGATCACC GCCCAACCTG ATGGCGGCGC AACCGAGAAC | 89 |
| | TGCGCGGTCC TG | |
| PstBssRev | CCCTGCAGCG CTTGTCGAAC CACTTGCCGT TGGCGGCGCC AGACAGGACC | 90 |
| C | GCGCAGTTCT | |
| Bglfor12 | GCCGAGATCT GGCTGGGCCT GAACGACATG | 91 |
| PstRev | ATCCCTGCAG CGCTTGTCGA ACC | 92 |
| Mu1Xfor | GCTGTTCGAA TACGCGCGCC ACAGCGTGGG CAACGATGCG AACATCTGGC | 93 |
| | TGGGCCTCAA CGATATG | |
| Mu1Xrev | GCCGCCGGTC ATGTCGACCC AMNNMNNMNN MNNMNNMNNM NNCATATCGT | 94 |
| | TGAGGCCCAG CCAG | |
| Mu1XSalFor | TGGGTCGACA TGACCGGCGG CNNKCTGGCC TACAAGAACT GGGAGACGGA | 95 |
| | GATCACGACG CAACCCGACG GCGGCGCTGC CGAGAACTG | |
| Mu1XPstRev | CAGCGTTTGT CGAACCACTT GCCGTTGGCT GCGCCAGACA GGGCGGCGCA | 96 |
| | GTTCTCGGCA GCGCCGCCGT CGGGTT | |
| BstBBssH | GCTGTTCGAA TACGCGCGCC ACAGCGTGG | 97 |
| Mu Pst | GGGCAACTGA TCTCTGCAGC GTTTGTCGAA CCACTTGCCG T | 98 |
| 1-2 for | GGCTGGGCCT GAACGACATG NNKNNKNNKN NKNNKTGGGT GGATATGNNK | 99 |
| | NNKNNKNNKA TCGCCTACAA GAACTGGGA | |
| 1-2 rev | GACAGGACGG CGCAGTTCTC GGTTGCGCCG CCATCAGGTT GGGCGGTGAT | 100 |
| | CTCAGTTTCC CAGTTCTTGT AGGCGAT | |
| PstRev12 | ATCCCTGCAG CGCTTGTCGA ACCACTTGCC GTTGGCGGCG CCAGACAGGA | 101 |
| | CGGCGCAGTT CTC | |
| Mu12rev | CGTCTCCCAG TTCTTGTAGG CCAGMNNMNN MNNMNNCATG TCGACCCAMN | 102 |
| | NMNNMNNMNN MNNCATATCG TTGAGGCCCA GCCAG | |
| Mu1234for | GCCTACAAGA ACTGGGAGAC GGAGATCACG ACGCAACCCG ACGGCGGCGC | 103 |
| | TGCCGAGAAC TG | |
| BglBssfor | GAGATCTGGC TCGCCTACAA CNNSNNSNNS NNSNNSNNSN NSTGGGTGGA | 104 |
| | CATGACTGGC | |
| BssBglrev | TTGCGCGGTG ATCTCAGTCT CCCAGTTCTT GTAGGCGATA CGCGCGCCAG | 105 |
| | TCATGTCCAC CCA | |
| BssPstfor | GACTGAGATC ACCGCGCAAC CCGATGGCGG CNNSNNSNNS NNSNNSGAGA | 106 |
| | ACTGCGCGGT CCTG | |
| PstBssRev | CCCTGCAGCG CTTGTCGAAC CACTTGCCGT TGGCCGCGCC TGACAGGACC | 107 |
| | GCGCAGTTCT | |
| Bglfor | GCCGAGATCT GGCTGGGCCT CA | 108 |
| MuUpsF | GCCATGGCCG CCTTACAGAC TGTGTGCCTG AAG | 109 |
| MuRanR | CGTCTCCCAG TTCTTGTAGG CCAGGAGGCC GCCGGTCATG TCCACCCAMN | 110 |
| | NMNNMNNMNN MNNMNNMNNG TTGAGGCCCA GCCAGAT | |
| MuRanF | GCCTACAAGA ACTGGGAGAC GGAGATCACG ACGCAACCCG ACGGCGGCNN | 111 |
| | KNNKNNKNNK NNKGAGAACT GCGCCGCCCT G | |
| MuDnsR | CGCACCTGCG GCCGCCACAA TGGCAAACTG GCAGATGT | 112 |
| H Loop 1- | ATCTGGCTGG GCCTGAACGA CATGGCCGCC GAGGGCACCT GGGTGGATAT | 113 |
| 2-F | GACCGGCGCG CGTATCGCCT ACAAGAAC | |
| H Loop 3- | CCGCCATCGG GTTGGGCMNN MNNMNNMNNM NNMNNAGTTT CCCAGTTCTT | 114 |
| 4 Ext R | GTAGGCGATA CG | |
| H Loop 3- | GCCCAACCCG ATGGCGGCNN KNNKNNKNNK NNKNNKAACT GCGCCGTCCT | 115 |
| 4 Ext-F | GTCTGGC | |
| H Loop 5- | CCTGCAGCGC TTGTCGAACC ACTTGCCGTT GGCGGCGCCA GACAGGACGG | 116 |
| R | CGCA | |
| M SacII-F | GACATGGCCG CGGAAGGCGC CTGGGTCGAC ATGACCGGCG GCCTGCTGGC | 117 |
| | CTACAAGAAC | |
| M Loop 3- | CCGCCGTCGG GTTGGGTMNN MNNMNNMNNM NNMNNGGTCT CCCAGTTCTT | 118 |
| 4 Ext-R | GTAGGCCAGC A | |
| M Loop 3- | ACCCAACCCG ACGGCGGCNN KNNKNNKNNK NNKNNKAACT GCGCCGCCCT | 119 |
| 4 Ext-F | GTCTGGC | |
| M Loop 5- | CTGATCTCTG CAGCGCTTGT CGAACCACTT GCCGTTGGCT GCGCCAGACA | 120 |
| R | GGGCGGCGCA GTT | |
| H Loop 3- | GCCAGACAGG ACGGCGCAGT TMNNMNNMNN GCCGCCMNNM NNMNNMNNMN | 121 |
| 4 Combo R | NMNNMNNMNN TTCCCAGTTC TTGTAGGCGA TACG | |
| M Loop 3- | GCCAGACAGG GCGGCGCAGT TMNNMNNMNN GCCGCCMNNM NNMNNMNNMN | 122 |
| 4 Combo R | NMNNMNNMNN CTCCCAGTTC TTGTAGGCCA GCA | |
| H Loop 3- | CCGCCATCGG GTTGGGCGGT GATCTCAGTT TCCCAGTTCT TGTAGGCGAT | 123 |
| R | ACG | |
| H Loop 4 | GCCCAACCCG ATGGCGGCNN KNNKNNKNNK NNKNNKNNKA ACTGCGCCGT | 124 |
| Ext-F | CCTGTCTGGC | |
| M Loop 3- | CCGCCGTCGG GTTGGGTGGT GATCTCGGTC TCCCAGTTCT TGTAGGCCAG | 125 |
| R | CA | |
| M Loop 4 | ACCCAACCCG ACGGCGGCNN KNNKNNKNNK NNKNNKNNKA ACTGCGCCGC | 126 |
| Ext-F | CCTGTCTGGC | |
| HLoop3F 6 | CTGGCGCGCG TATCGCCTAC AAGAACTGGN NKNNKNNKNN KNNKNNKCAA | 127 |
| | CCCGATGGCG GCGCCACCGA GAAC | |
| HLoop3F 7 | CTGGCGCGCG TATCGCCTAC AAGAACTGGN NKNNKNNKNN KNNKNNKNNK | 128 |
| | CAACCCGATG GCGGCGCCAC CGAGAAC | |
| HLoop3F 8 | CTGGCGCGCG TATCGCCTAC AAGAACTGGN NKNNKNNKNN KNNKNNKNNK | 129 |
| | CAACCCGATG GCGGCGCCAC CGAGAAC | |
| HLoop4R | CCTGCAGCGC TTGTCGAACC ACTTGCCGTT GGCGGCGCCA GACAGGACGG | 130 |
| | CGCAGTTCTC GGTGGCGCCG CCATCGGGTT G | |
| MLoop3F 6 | GTTCTCGGCA GCGCCGCCGT CGGGTTGMNN MNNMNNMNNM NNMNNCCAGT | 131 |
| | TCTTGTAGGC CAGCAGGCCG CCGGTCA | |
| HLoop3F 7 | GTTCTCGGCA GCGCCGCCGT CGGGTTGMNN MNNMNNMNNM NNMNNMNNCC | 133 |
| | AGTTCTTGTA GGCCAGCAGG CCGCCGGTCA | |
| MLoop3F 8 | GTTCTCGGCA GCGCCGCCGT CGGGTTGMNN MNNMNNMNNM NNMNNMNNMN | 134 |
| | NCCAGTTCTT GTAGGCCAGC AGGCCGCCGG TCA | |
| M 3X OF | GACATGGCCGCGGAAGGC | 135 |
| H1-3-4R | GACAGGACCG CGCAGTTCTC GCCSMAGWMC CCSAAGCCGC CMNNGGGTTG | 136 |
| | MNNMNNMNNM NNMNNCTCCC AGTTCTTGTA GGCGATACG | |
| PstLoop4 | ATCCCTGCAG CGCTTGTCGA ACCACTTGCC GTTGGCCGCG CCTGACAGGA | 137 |
| rev | CCGCGCAGTT CTCGCC | |
| Loop3AF2 | GAGCGTGGGCAACGAGGCCGAGATCTGGCTGGGCCTCAACGACATGGCCGCCGA | 138 |
| Loop3AR2 | CCAGTTCTTGTAGGCGATACGCGCGCCAGTCATATCCACCCAGGTGCCCTCGGC | 139 |
| | GGCCATGTCGTTGAGG | |
| Loop3BF | ATCGCCTACAAGAACTGGGAGACTGRGNNKNNKNNKNNKNNKNNKNNKACCGCG | 140 |
| | CAACCCGATGGCGGTGCAAC | |
| Loop3BR | CGCTTGTCGAACCACTTGCCGTTGGCGGCGCCAGACAGGACGGCGCAGTTCTCG | 141 |
| | GTTGCACCGCCATCGGGTTG | |
| Loop3OR | GATCCCTGCAGCGCTTGTCGAACCACTTGCCGT | 142 |
| M 3X OR | GCAGATGTAGGGCAACTGATCTCT | 143 |
| HuBglfor | GCCGAGATCTGGCTGGGCCTGA | 144 |
| GSXX | GCCGAGATCTGGCTGGGCCTCAACGGCAGCNNKNNKNNKNNKWCCTGGGTGGAC | 145 |
| | ATGACTGGC | |
| 090827 | TTGCGCGGTGATCTCAGTCTCCCAGTTCTTGTAGGCGATACGCGCGCCAGTCAT | 146 |
| BssBglrev | GTCCACCCA | |
| FGVFGfor | GACTGAGATCACCGCGCAACCCGATGGCGGCTTCGGCGTGTTCGGCGAGAACTG | 147 |
| | CGCGGTCCTG | |
| WGVFGfor | GACTGAGATCACCGCGCAACCCGATGGCGGCTGGGGCGTGTTCGGCGAGAACTG | 148 |
| | CGCGGTCCTG | |
| FGYFGfor | GACTGAGATCACCGCGCAACCCGATGGCGGCTTCGGGTACTTCGGCGAGAACTG | 149 |
| | CGCGGTCCTG | |
| WGYFGfor | GACTGAGATCACCGCGCAACCCGATGGCGGCTGGGGGTACTTCGGCGAGAACTG | 150 |
| | CGCGGTCCTG | |
| WGVWGfor | GACTGAGATCACCGCGCAACCCGATGGCGGCTGGGGCGTGTGGGGCGAGAACTG | 151 |
| | CGCGGTCCTG | |
| Mu 1-4 AF | GGCAACGATGCGAACATCTGGCTGGGCCTCAACNNKNNKNNKNNKNNKNNKNNK | 152 |
| | TGGGTCGACATGACCGGC | |
| Mu 1-4 AR | GGTTGCGTCGTGATCTCCGTCTCCCAGTTCTTGTAGGCCAGGAGGCCGCCGGTC | 153 |
| | ATGTCGACCCA | |
| Mu 1-4 BF | GACGGAGATCACGACGCAACCCGACGGCGGCNNKNNKNNKNNKNNKGAGAACTG | 154 |
| | TGCTGCCCTGTCTGG | |
| Mu 1-4 BR | CTCTGCAGCGCTTGTCGAACCACTTGCCGTTGGCTGCGCCAGACAGGGCAGCAC | 155 |
| | AGTTCTC | |
| Mu 1-4 OF | ATACGCGCGCCACAGCGTGGGCAACGATGCGAACATCTG | 156 |
| Mu 1-4 OR | ATCTCTGCAGCGCTTGTCGAACC | 157 |
| Mloop4F | CAACCCGACGGCGGCGCTGCCGAGAACTGCGCCGCCCTGTCTGGCGCAGCCAAC | 158 |
| | GGCAAGTG | |
| M MfeR | GCAGATGTAGGGCAACTGATCTCTGCAGCGCTTGTCGAACCACTTGCCGTTGGC | 159 |
| | TGCGCCAGAC | |
| m3-5 for | GCTGGCCTACAAGAACTGGGAGNNKNNKNNKNNKNNKCAACCCGACGGCGGCGC | 160 |
| | AGCTGAGAACTG | |
| m3-5 rev | GCGCTTGTCGAACCACTTGCCMNNMNNMNNGCCAGACAGGGCGGCGCAGTTCTC | 161 |
| | AGCTGCGCCGCCGT | |
| m3-5 OF | CTGGGTCGACATGACCGGCGGCCTGCTGGCCTACAAGAACTGGGAG | 162 |
| m3-5 OR | ATCTCTGCAGCGCTTGTCGAACCACTTG | 163 |
| h3-5AF | TGGGCCTGAACGACATGGCCGCCGAGGGCACCTGGGTGGATATGACTGGCGCGC | 164 |
| | GTATCGCCTACAAGAACTGGGAG | |
| h3-5AR | GTTGCGCCGCCATCGGGTTGMNNMNNMNNMNNMNNCTCCCAGTTCTTGTAGGCG | 165 |
| | ATACG | |
| h3-5BF | CAACCCGATGGCGGCGCAACCGAGAACTGCGCCGTCCTGTCTGG | |
| h3-5BR | TGTAGGGCAATTGATCCCTGCAGCGCTTGTCGAACCACTTGCCMNNMNNMNNGC | 166 |
| | CAGACAGGACGGCGCAGTT | |
| h3-5 OF | GCCGAGATCTGGCTGGGCCTGAACGACATGG | 167 |

M = A or C; N = A, C, G, or T; K = G or T; S = G or C; W = A or T.

### Example 4

Library Construction: Mutation and Extension of Loop 1

The sequences of human tetranectin and mouse tetranectin, and the positions of loops 1, 2, 3, 4 (LSA) and 5 (LSB) are shown in FIGS. 6, 7 and 10. For the 1-2 extended libraries of human and mouse tetranectin C-type lectin binding domains (“Human 1X-2” and “Mouse 1X-2,” respectively), the coding sequences for Loop 1 are modified to encode the sequences shown in Table 4, where the five amino acids AAEGT (SEQ ID NO: 207; human) or AAEGA ((SEQ ID NO: 208; mouse) are substituted with seven random amino acids encoded by the nucleotides NNK NNK NNK NNK NNK NNK NNK (SEQ ID NO: 20); N denotes A, C, G, or T; K denotes G or T. The amino acid arginine immediately following Loop 2 is also fully randomized by using the nucleotides NNK in the coding strand. This amino acid is randomized because the arginine contacts amino acids in Loop 1, and might constrain the configurations attainable by Loop 1 randomization. In addition, the coding sequence for Loop 4 is altered to encode an alanine (A) instead of Lysine 148 (K) in order to abrogate plasminogen binding, which has been shown to be dependent on the Loop 4 lysine (Graversen et al., 1998). The sequences of human tetranectin and mouse tetranectin, and the positions of Loops 1, 2, 3, 4, and 5 are shown in FIG. 10.

TABLE 5
Amino acids of loop regions from human and mouse tetranectin (TN).

| | | Loop 2 | | | |
|---|---|---|---|---|---|
| | Loop 1 | [SEQ ID | Loop 3 | Loop 4 | Loop |
| Library | [SEQ ID NO] | NO] | [SEQ ID NO] | [SEQ ID NO] | 5 |
| Human | DMAAEGTW | DMTGA(R) | NWETEITAQ(P) | DGGKTEN | AAN |
| TN | [65] | [73] | [75] | [83] | |
| Human | DMXXXXXXXW | DMTGA(X) | NWETEITAQ(P) | DGGATEN | AAN |
| 1X-2 | [66] | [74] | [75] | [83] | |
| Human | DMXXXXXW | DMXXX(X) | NWETEITAQ(P) | DGGATEN | AAN |
| 1-2 | [67] | [71] | [75] | [83] | |
| Human | XXXXXXXW | DMTGA(R) | NWETEITAQ(P) | DGGXXXXXEN | AAN |
| 1-4 | [68] | [73] | [75] | [84] | |
| Human | DMAAEGTW | DMTGA(R) | NWXXXXXXQ(P) | DGGATEN | AAN |
| 3X 6 | [84] | [73] | [76] | [83] | |
| Human | DMAAEGTW | DMTGA(R) | NWXXXXXXXQ(P) | DGGATEN | AAN |
| 3X 7 | [65] | [73] | [77] | [83] | |
| Human | DMAAEGTW | DMTGA(R) | NWXXXXXXXXQ(P) | DGGATEN | AAN |
| 3X 8 | [65] | [73] | [78] | [83] | |
| Human | DMAAEGTW | DMTGA(R) | NWETEXXXXXXXTAQ(P) | DGGATEN | AAN |
| 3X loop | [65] | [73] | [79] | [83] | |
| Human | DMAAEGTW | DMTGA(R) | NWETXXXXXXAQ(P) | DGGATEN | AAN |
| 3-4X | [65] | [73] | [78] | [85] | |
| Human | DMAAEGTW | DMTGA(R) | NWEXXXXXX(X) | XGGXXXN | AAN |
| 3-4 | [65] | [73] | [80] | [87] | |
| combo | | | | | |
| Human | DMAAEGTW | DMTGA(R) | NWEXXXXXQ(P) | DGGATEN | XXX |
| 3-5 | [65] | [73] | [76] | [83] | |
| Human | DMAAEGTW | DMTGA(R) | NWETEITAQ(P) | DGGXXXXXXXN | AAN |
| 4 | [65] | [73] | [75] | [85] | |
| Mouse | DMAAEGAW | DMTGG(L) | NWETEITTQ(P) | DGGKAEN | AAN |
| TN | [69] | [70] | [75] | [86] | |
| Mouse | DMXXXXXXXW | DMTGG(X) | NWETEITTQ(P) | DGGAAEN | AAN |
| 1X-2 | [66] | [72] | [75] | [184] | |
| Mouse | DMXXXXXW | DMXXX(X) | NWETEITTQ(P) | DGGAAEN | AAN |
| 1-2 | [67] | [71] | [75] | [184] | |
| Mouse | XXXXXXXW | DMTGG(L) | NWETEITTQ(P) | DGGXXXXXEN | AAN |
| 1-4 | [68] | [70] | [75] | [84] | |
| Mouse | DMAAEGAW | DMTGG(L) | NWXXXXXXQ(P) | DGGKAEN | AAN |
| 3X | [69] | [70] | [76] | [86] | |
| Mouse | DMAAEGAW | DMTGG(L) | NWXXXXXXXQ(P) | DGGKAEN | AAN |
| 3X | [69] | [70] | [77] | [86] | |
| Mouse | DMAAEGAW | DMTGG(L) | NWXXXXXXXXQ(P) | DGGKAEN | AAN |
| 3X | [69] | [70] | [78] | [86] | |
| Mouse | DMAAEGAW | DMTGG(L) | NWETEXXXXXXXTTQ(P) | DGGKAEN | AAN |
| 3X loop | [69] | [70] | [81] | [86] | |
| Mouse | DMAAEGAW | DMTGG(L) | NWETXXXXXXTQ(P) | DGGXXXXXXN | AAN |
| 3-4X | [69] | [70] | [82] | [85] | |
| Mouse | DMAAEGAW | DMTGG(L) | NWEXXXXXX(X) | XGGXXXN | AAN |
| 3-4 | [69] | [70] | [80] | [87] | |
| combo | | | | | |
| Mouse | DMAAEGAW | DMTGG(L) | NWEXXXXXQ(P) | DGGKAEN | XXX |
| 3-5 | [69] | [70] | [76] | [86] | |
| Mouse | DMAAEGAW | DMTGG(L) | NWETEITTQ(P) | DGGXXXXXXXN | AAN |
| 4 | [69] | [70] | [75] | [85] | |

Parentheses indicate neighboring amino acids not considered part of the loop.
X = any amino acid.

The human Loop 1 extended library can be generated using overlap PCR in the following manner (all primer sequences are shown in Table 4). Primers 1Xfor and 1Xrev are mixed and extended by PCR, and primers BstX1for and PstBssRevC are mixed and extended by PCR. The resulting fragments are purified from gels, and mixed and extended by PCR in the presence of the outer primers Bg1for12 and PstRev. The resulting fragment is gel purified and cut with Bgl II and Pst I and cloned into a phage display vector pPhCPAB or pANA27.

Ligated material is transformed into electrocompetent XL1-Blue E. coil (Stratagene) and four to eight liters of cells are grown overnight and DNA isolated to generate a master library DNA stock for panning. A library size of 1.5×10⁸ is obtained, and clones examined showed diversified sequence in the targeted regions.

The mouse Loop 1 extended library is generated using overlap PCR in the following manner. Primers Mu1Xfor and Mu1Xrev are mixed and extended by PCR, and primers Mu1XSal1for and Mu1XPstRev are mixed and extended by PCR. The resulting fragments are purified from gels, mixed and extended by PCR in the presence of the outer primers BstBBssH and Mu Pst. The resulting fragment is gel purified and cut with BssH II and Pst I and ligated into similarly digested phage display vector pANA16 or pANA28. Phage display vector pANA16 is derived from pPhCPAB by replacing the human tetranectin CTLD with the mouse tetranectin CTLD. The mouse tetranectin CTLD included BstBI, BssHII, and SalI sites within the Loop 1-4 region and a PstI site after the Loop 4 region similar to pPhCPAB in order to facilitate cloning. In addition, the region is altered to include stop codons as described above. Phage display vector pANA28 is derived from pANA16 by replacing the BamHI to ClaI region with the BamHI to ClaI sequence. Ligated material is transformed into electrocompetent XL1-Blue E. coli (Stratagene) and four to eight liters of cells are grown overnight and DNA isolated to generate a master library DNA stock for panning. A library size of 2.65×10¹⁰ is obtained, and clones examined showed diversified sequence in the targeted regions.

### Example 5

Library Construction: Mutation of Loops 1 and 2

For the Loop 1-2 libraries of human and mouse tetranectin C-type lectin binding domains (“Human 1-2” and “Mouse 1-2,” respectively), the coding sequences for Loop 1 are modified to encode the sequences shown in Table 1, where the five amino acids AAEGT (SEQ ID NO: 171; human) or AAEGA (SEQ ID NO: 172; mouse) are replaced with five random amino acids encoded by the nucleotides NNK NNK NNK NNK NNK ((SEQ ID NO: 178); N denotes A, C, G, or T; K denotes G or T). In Loop 2 (including the neighboring arginine), the four amino acids TGAR in human or TGGR in mouse are replaced with four random amino acids encoded by the nucleotides NNK NNK NNK NNK (SEQ ID NO: 178). In addition, the coding sequence for Loop 4 is altered to encode an alanine (A) instead of the lysine (K) in the loop, in order to abrogate plasminogen binding, which has been shown to be dependent on the Loop 4 lysine (Graversen et al., 1998).

The human 1-2 library is generated using overlap PCR in the following manner (primer sequences are shown in Table 4). Primers 1-2 for and 1-2 rev are mixed and extended by PCR. The resulting fragment is purified from gels, mixed and extended by PCR in the presence of the outer primers Bglfor12 and PstRev12. The resulting fragment is gel purified and cut with Bgl II and Pst I and cloned into similarly digested phage display vector pPhCPAB or pANA27, as described above. A library size of 4.86×10⁸ is obtained, and clones examined showed diversified sequence in the targeted regions.

The mouse Loop 1-2 library is generated using overlap PCR in the following manner. Primers Mu1Xfor and Mu12rev are mixed and extended by PCR, and primers Mu1234for and Mu1XPstRev are mixed and extended by PCR. The resulting fragments are purified from gels, mixed and extended by PCR in the presence of the outer primers BstBBssH and Mu Pst. The resulting fragment is gel purified and cut with BssH II and Pst I and cloned into similarly digested phage display vector pANA16 or pANA28, as described above. A library size of 1.63×10⁹ is obtained, and clones examined showed diversified sequence in the targeted regions.

### Example 6

Library Construction: Mutation and Extension of Loops 1 and 4

For the Loop 1-4 libraries of human and mouse tetranectin C-type lectin binding domains (“Human 1-4” and “Mouse 1-4,” respectively), the coding sequences for Loop 1 are modified to encode the sequences shown in Table 4, where the seven amino acids DMAAEGT (see SEQ ID NO: 185; human) or DMAAEGA (see SEQ ID NO: 186; mouse) are replaced with seven random amino acids encoded by the nucleotides NNK NNK NNK NNK NNK NNK((SEQ ID NO: 180); N denotes A, C, G, or T; K denotes G or T). In Loop 4 two amino acids KT in human or KA in mouse, are replaced with five random amino acids encoded by the nucleotides NNK NNK NNK NNK NNK(SEQ ID NO: 178).

The human 1-4 library is generated using overlap PCR in the following manner (primer sequences are shown in Table 4). Primers BglBssfor and BssBglrev are mixed and extended by PCR, and primers BssPstfor and PstBssRev are mixed and extended by PCR. The resulting fragments are purified from gels, mixed and extended by PCR in the presence of the outer primers Bglfor and PstRev. The resulting fragment is gel purified and cut with Bgl II and Pst I restriction enzymes, and cloned into similarly digested phage display vector pPhCPAB or pANA27, as described above. A library size of 2×10⁹ is obtained, and12 clones examined prior to panning showed diversified sequence in the targeted regions.

The mouse 1-4 library is generated using overlap PCR in the following manner (primer sequences are shown in Table 4). Primers Mu 1-4 AF and Mu 1-4 AR are mixed and extended by PCR, and primers Mu 1-4 BF and Mu 1-4 BR are mixed and extended by PCR. The resulting fragments are purified from gels, mixed and extended by PCR in the presence of the outer primers Mu 1-4 OF and Mu 1-4 OR. The resulting fragment is gel purified and cut with BstB I and Pst I restriction enzymes, and cloned into similarly digested phage display vector pANA28, as described above. A library size of 4.7×10⁹ is obtained, and >20 clones are examined prior to panning showed diversified sequence in the targeted regions.

### Example 7

Library Construction: Mutation and Extension of Loops 3 and 4

For the Loop 3-4 extended libraries of human and mouse tetranectin C-type lectin binding domains (“Human 3-4X” and “Mouse 3-4X,” respectively), the coding sequences for Loop 3 are modified to encode the sequences shown in Table 4, where the three amino acids EIT of human or mouse tetranectin are replaced with six random amino acids encoded by the nucleotides NNK NNK NNK NNK NNK NNK (SEQ ID NO: 180) in the coding strand (N denotes A, C, G, or T; K denotes G or T). In addition, in Loop 4, the three amino acids KTE in human or KAE in mouse are replaced with six random amino acids encoded by the nucleotides NNK NNK NNK NNK NNK NNK (SEQ ID NO: 180).

The human 3-4 extended library is generated using overlap PCR in the following manner (primer sequences are shown in Table 4). Primers H Loop 1-2-F and H Loop 3-4 Ext-R are mixed and extended by PCR, and primers H Loop 3-4 Ext-F and H Loop 5-R are mixed and extended by PCR. The resulting fragments are purified from gels, and mixed and extended by PCR in the presence of additional H Loop 1-2-F and H Loop 5-R. The resulting fragment is gel purified and cut with Bgl II and Pst I restriction enzymes, and cloned into similarly digested phage display vector pPhCPAB or pANA27, as described above. A library size of 7.9×10⁸ is obtained, and clones examined showed diversified sequence in the targeted regions.

The mouse 3-4 extended library is generated using overlap PCR in the following manner. Primers M SacII-F and M Loop 3-4 Ext-R are mixed and extended by PCR, and primers M Loop 3-4 Ext-F and M Loop 5-R are mixed and extended by PCR. The resulting fragments are purified from gels, and mixed and extended by PCR in the presence of additional M SacII-F and M Loop 5-R. The resulting fragment is gel purified and cut with Sac II and Pst I restriction enzymes, and cloned into similarly digested phage display vector pANA16 or pANA28, as described above. A library size of 4.95×10⁹ is obtained, and clones examined showed diversified sequence in the targeted regions.

### Example 8

Library Construction: Mutation of Loops 3 and 4 and the PRO Between the Loops

For the Loop 3-4 combo library of human and mouse tetranectin C-type lectin binding domains (“Human 3-4 combo” and “Mouse 3-4 combo,” respectively), the coding sequences for loops 3 and 4 and the proline between these two loops are altered to encode the sequences shown in Table 5, where the human sequence TEITAQPDGGKTE (SEQ ID NO: 187) or the corresponding mouse sequence TEITTQPDGGKAE (SEQ ID NO: 188) are replaced by the 13 amino acid sequence XXXXXXXXGGXXX, (SEQ ID NO: 189) where X represents a random amino acid encoded by the sequence NNK (N denotes A, C, G, or T; K denotes G or T).

The human 3-4 combo library is generated using overlap PCR in the following manner (primer sequences are shown in Table 4). Primers H Loop 1-2-F and H Loop 3-4 Combo-R are mixed and extended by PCR and the resulting fragment is purified from gels and mixed and extended by PCR in the presence of additional H Loop 1-2-F and H loop 5-R. The resulting fragment is gel purified and cut with Bgl II and Pst I restriction enzymes, and cloned into similarly digested phage display vector pPhCPAB or pANA27, as described above. A library size of 4.95×10⁹ is obtained, and clones examined showed diversified sequence in the targeted regions.

The mouse 3-4 combo library is generated using overlap PCR in the following manner. Primers M SacII-F and M Loop 3-4 Combo-R are mixed and extended by PCR and the resulting fragment is purified from gels and mixed and extended by PCR in the presence of the outer primers M SacII-F and M Loop 5-R. The resulting fragment is gel purified and cut with Sac II and Pst I restriction enzymes, and cloned into similarly digested phage display vector pANA16 or pANA28, as described above. A library size of 7.29×10⁸ is obtained, and clones examined showed diversified sequence in the targeted regions.

### Example 9

Library Construction: Mutation and Extension of Loop 4

For the Loop 4 extended libraries of human and mouse tetranectin C-type lectin binding domains (“Human 4” and “Mouse 4,” respectively), the coding sequences for Loop 4 are modified to encode the sequences shown in Table 4, where the three amino acids KTE of human or KAE of mouse tetranectin are replaced with seven random amino acids encoded by the nucleotides NNK NNK NNK NNK NNK NNK NNK ((SEQ ID NO: 20); N denotes A, C, G, or T; K denotes G or T).

The human 4 extended library is generated using overlap PCR in the following manner (primer sequences are shown in Table 4). Primers H Loop 1-2-F and H Loop 3-R are mixed and extended by PCR, and primers H Loop 4 Ext-F and H Loop 5-R are mixed and extended by PCR. The resulting fragments are purified from gels, and mixed and extended by PCR in the presence of additional H Loop 1-2-F and H Loop 5-R. The resulting fragment gel purified and is cut with Bgl II and Pst I restriction enzymes, and cloned into similarly digested phage display vector pPhCPAB or pANA27, as described above. A library size of 2.7×10⁹ is obtained, and clones examined showed diversified sequence in the targeted regions.

The mouse 4 extended library is generated using overlap PCR in the following manner. Primers M SacII-F and M Loop 3-R are mixed and extended by PCR, and primers M Loop 4 Ext-F and M Loop 5-R are mixed and extended by PCR. The resulting fragments are purified from gels, and mixed and extended by PCR in the presence of the additional M and M Loop 5-R. The resulting fragment is gel purified, digested with SacII and PstI restriction enzymes, and cloned into similarly digested phage display vector pANA16 or pANA28, as described above.

Example 10

Library Construction: Mutation with and without Extension of Loop 3

For the Loop 3 altered libraries of human and mouse tetranectin C-type lectin binding domains, the coding sequences for Loop 3 are modified to encode the sequences shown in Table 5, where the six amino acids ETEITA (SEQ ID NO: 190) of human or ETEITT (SEQ ID NO: 191) of mouse tetranectin are replaced with six, seven, or eight random amino acids encoded by the nucleotides NNK NNK NNK NNK NNK NNK (SEQ ID NO: 180), NNK NNK NNK NNK NNK NNK NNK (SEQ ID NO: 20), and NNK NNK NNK NNK NNK NNK NNK NNK (SEQ ID NO: 181); N denotes A, C, G, or T; and K denotes G or T. In addition, in Loop 4, the three amino acids KTE in human or KAE in mouse are replaced with six random amino acids encoded by the nucleotides NNK NNK NNK NNK NNK NNK (SEQ ID NO: 180). In addition the coding sequence for loop 4 is altered to encode an alanine (A) instead of the lysine (K) in the loop, in order to abrogate plasminogen binding, which has been shown to be dependent on the loop 4 lysine (Graversen et al., 1998).

The human Loop 3 altered library is generated using overlap PCR in the following manner. Primers HLoop3F6, HLoop3F7, and HLoop3F8 are individually mixed with HLoop4R and extended by PCR. The resulting fragments are purified from gels, and mixed and extended by PCR in the presence of oligos H Loop 1-2F, Bglfor and PstRev. The resulting fragments are gel purified, digested with BglI and PstI restriction enzymes, and cloned into similarly digested phage display vector pPhCPAB or pANA27, as above. After library generation, the three libraries are pooled for panning.

The mouse Loop 3 altered library is generated using overlap PCR in the following manner. Primers MLoop3F 6, MLoop3F 7, and MLoop3F 8 are individually mixed with primer M SacII-F and extended by PCR. In addition, primers MLoop4F and M MfeR are mixed and extended by PCR. The resulting fragments are purified from gels, mixed, and subjected to PCR in the presence of primers M 3X OF and M 3X OR. Products are digested with Sal I (or Sac II) and PstI restriction enzymes, and the purified fragments are cloned into similarly digested phage display vector pANA16 or pANA28, as described above.

Alternate loop extension of loop 3

The human loop 3 loop library is generated using overlap PCR in the following manner. Primers Loop3AF2 and Loop3AR2 are mixed and extended by PCR, and primers Loop3BF and Loop3BR are mixed and extended by PCR. The resulting fragments are purified from gels, mixed, and subjected to PCR in the presence of primers Bglfor and Loop3OR. Products are digested with Bgl II and Pst I restriction enzymes, and the purified fragments are cloned into similarly digested phage display vector pPhCPAB or pANA27, as above. In addition the coding sequence for loop 4 is altered to encode an alanine (A) instead of the lysine (K) in the loop, in order to abrogate plasminogen binding, which has been shown to be dependent on the loop 4 lysine (Graversen et al., 1998). A similar approach can be used to generate the corresponding mouse TN library.

### Example 11

Mutation of Loops 3 and 5

For the loop 3 and 5 altered libraries of human and mouse tetranectin C-type lectin binding domains, the coding sequences for loops 3 and 5 are modified to encode the sequences shown in Table 5, where the five amino acids TEITA of human or TEITT of mouse tetranectin are replaced with five amino acids encoded by the nucleotides NNK NNK NNK NNK NNK (SEQ ID NO: 180), and the three Loop 5 amino acids AAN of human or mouse are replaced with three amino acids encoded by the nucleotides NNK NNK NNK. In addition the coding sequence for loop 4 is altered to encode an alanine (A) instead of the lysine (K) in the loop, in order to abrogate plasminogen binding, which has been shown to be dependent o n the loop 4 lysine (Graversen et al., 1998).

The human loop 3 and 5 altered library is generated using overlap PCR in the following manner. Primers h3-5AF and h3-5AR are mixed and extended by PCR, and primers h3-5BF and h3-5 BR are mixed and extended by PCR. The resulting fragments are purified from gels, and mixed and extended by PCR in the presence of h3-5 OF and PstRev. The resulting fragment is gel purified, digested with Bgl I and Pst I restriction enzymes, and cloned into similarly digested phage display vector pPhCPAB or pANA27 as described above.

The mouse loop 3 and 5 altered library is generated using overlap PCR in the following manner. Primers m3-5 for and m3-5 rev are mixed and extended by PCR. The resulting fragment is purified from gels, and reamplified by PCR with primers m3-5 OF and m3-5 OR. Products are digested with Sal I and Pst I restriction enzymes, and the purified fragments are cloned into similarly digested phage display vector pANA16 or pANA28 as described above.

Examples 13-24 provide prophetic exemplary methods for isolating polypeptide sequences specific for HSP70 using the combinatorial polypeptide libraries of the invention. Accordingly, the CTLD polypeptide libraries of the invention are screened in an effort to identify and isolate CTLD-based polypeptides having specific binding activity to HSP70.

### Example 12

Panning & Screening of Human Library 1-4

Phage generated from human library 1-4 are panned on recombinant HSP70/Fc chimera. Screening of these binding panels after three, four, and/or five rounds of panning using an ELISA plate assay can identify receptor-specific binders in many cases.

### Example 13

Construction of Libraries and Clones for Selection and Screening of Binding Polypeptides for HSP70

Phage libraries expressing linear or cyclized randomized peptides of varying lengths can be purchased commercially from manufacturers such as New England Biolabs (NEB). Alternatively, phage display libraries containing randomized peptides in loops of the C-type lectin domain (CTLD) of human tetranectin can be generated. Loops 1, 2, 3, and 4 of the LSA of CTLD are shown in FIG. 7. Amino acids within these loops can be randomized using an NNS or NNK overlapping PCR mutagenesis strategy. From one to seven codons in any one loop may be replaced by a mutagenic NNS or NNK codon to generate libraries for screening; alternatively, the number of mutagenized amino acids may exceed the number being replaced (two amino acids may be replaced by five, for example, to make larger randomized loops). In addition, more than one loop may be altered at the same time. The overlap PCR strategy can generate either a Kpn I site in the final DNA construct between loops 2 and 3, which alters one of the amino acids between the loops, exchanging a threonine for the original alanine. Alternatively, a BssH II site can be incorporated between loops 2 and 3 that does not alter the original amino acid sequence.

### Example 14

Plasmid Construction of Trimeric HSP70 Binding Polypeptides and Trimeric CTLD-Derived HSP70 Binding Polypeptides

The various versions of trimeric HSP70 binding polypeptides and trimeric CTLD-derived HSP70 binding polypeptides from phage display or from peptide-grafted, peptide-trimerization domain (TD) fusions, peptide-TD-CTLD fusion, or their various combinations are sub-cloned into bacterial expression vectors (pT7 in house vector, or pET, NovaGen) and mammalian expression vectors (pCEP4, pcDNA3, Invitrogen) for small scale or large-scale production.

Primers are designed to PCR amplify DNA fragments of binders from various functional display vectors from as described herein. Primers for the 5′-end are flanked with BamH I restriction sites and are in frame with the leader sequence in the vector pT7CIIH6. 5′ primers also can be incorporated with a cleavage site for protease Granzyme B or Factor Xa. 3′ primers are flanked with EcoRI restriction sites. PCR products are digested with BamHI/EcoRI, and then ligated into pT7CIIH6 digested with the same enzymes, to create bacterial expression vectors pT7CIIH6-HSP70a.

The HSP70 binding polypeptide DNAs can be sub-cloned into vector pT7CIIH6 or pET28a (NovoGen), without any leader sequences and 6× His. 5′ primers are flanked with NdeI restriction sites and 3′ primers are flanked with EcoRI restriction sites. PCR products are digested with NdeI/EcoRI, and ligated into the vectors digested with the same enzymes, to create expression vectors pT7-HSP70a and pET-HSP70a.

The HSP70 agonist DNAs can be sub-cloned into vector pT7CIIH6 or pET28a (NovoGen), with a secretion signal peptide. Expressed proteins are exported into bacterial periplasm, and secretion signal peptide is removed during translocation. 5′ primers are flanked with NdeI restriction sites and the primers are incorporated into a bacterial secretion signal peptide, PelB, OmpA or OmpT. 3′ primers are flanked with EcoRI restriction sites. A 633 His tag coding sequence can optionally be incorporated into the 3′ primers. PCR products are digested with NdeI/EcoRI, and ligated into vectors that are digested with the same enzymes, to create the expression vectors pT7-s HSP70a, pET-sHSP70a, pT7-s HSP70aHis, and pET-s HSP70His.

The HSP70 agonist DNAs can also be sub-cloned into mammalian expression vector pCEP4 or pcDNA3.1, along with a secretion signal peptide. Expressed proteins are secreted into the culture medium, and the secretion signal peptide is removed during the secretion processes. 5′ primers are flanked with NheI restriction sites and the primers are incorporated into a tetranectin secretion signal peptide, or another secretion signal peptide (e.g., Ig peptide). 3′ primers are flanked with XhoI restriction sites. A 6× His tag is optionally incorporated into the 3′ primers. PCR products are digested with NheI/XhoI, and ligated into the vectors that are digested with the same enzymes, to create expression vectors pCEP4-HSP70a, pcDNA-HSP70a, pCEP4-HSP70aHis, and pcDNA-HSP70aHis.

### Example 15

Expression and Purification of HSP70 Binding Polypeptides from Bacteria

Bacterial expression constructs can be transformed into bacterial strain BL21(DE3) (Invitrogen). A single colony on a fresh plate is inoculated into 100 mL of 2×YT medium in a shaker flask. The flask is incubated in a shaker rotating at 250 rpm at 37° C. for 12 h or overnight. Overnight culture (50 mL) is used to inoculate 1 L of 2×YT in a 4 L shaker flask. Bacteria are cultured in the flask to an OD₆₀₀ of about 0.7, at which time IPTG is added to the culture to a final concentration of 1 mM. After a 4 h induction, bacterial pellets are collected by centrifugation and saved for subsequent protein purification.

Bacterial fermentation is performed under fed-batch conditions in a 10-liter fermentor. One liter of complex fermentation medium contains 5 g of yeast extract, 20 g of tryptone, 0.5 g of NaCl, 4.25 g of KH₂PO₄, 4.25 g of K₂HPO₄.3H₂O, 8 g of glucose, 2 g of MgSO₄.7H₂O, and 3 mL of trace metal solution (2.7% FeCl₃.6H₂O/0.2% ZnCl₂.4H₂O/0.2% CoCl₂.6H₂O/0.15% Na₂MoO₄.2H₂O/0.1% CaCl₂.2H₂O/0.1% CuCl₂/0.05% H₃BO₃/3.7% HCl). The fermentor is inoculated with an overnight culture (5% vol/vol) and grown at constant operating conditions at pH 6.9 (controlled with ammonium hydroxide and phosphoric acid) and at 30° C. The airflow rate and agitation are varied to maintain a minimum dissolved oxygen level of 40%. The feed (with 40% glucose) is initiated once the glucose level in the culture is below 1 g/L, and the glucose level is maintained at 0.5 g/L for the rest of the feimentation. When the OD₆₀₀ reaches about 60, TTG is added into the culture to a final concentration of 0.05 mM. Four hours after induction, the cells are harvested. The bacterial pellet is obtained by centrifugation and stored at −80° C. for subsequent protein purification.

Expressed proteins that are soluble, secreted into the periplasm of the bacterial cell, and include an affinity tag (e.g., 6× His tagged proteins) are purified using standard chromatographic methods, such as metal chelation chromatography (e.g., Ni affinity column), anionic/cationic affinity chromatography, size exclusion chromatography, or any combination thereof, which are well known to one skilled in the art.

Expressed proteins can form insoluble inclusion bodies in bacterial cells. These proteins are purified under denaturing conditions in initial purification steps and undergo a subsequent refolding procedure, which can be performed on a purification chromatography column. The bacterial pellets are suspended in a lysis buffer (0.5 M NaCl, 10 mM Tris-HCl, pH 8, and 1 mM EDTA) and sonicated. The inclusion body is recovered by centrifugation, and subsequently dissolved in a binding buffer containing 6M guanidinium chloride, 50 mM Tri-HCl, pH8, and 0.1M DTT. The solubilized portion is applied to a Ni affinitycolumn. After washing the unbound materials from the column, the proteins are eluted with an elution buffer (6M guanidinium chloride, 50 mM Tris-HCl pH8.0, 10 mM 2-mercaptoethanol, 250 mM imidazole). Isolated proteins are buffer exchanged into the binding buffer, and are re-applied to the Ni⁺ column to remove the denaturing agent. Once loaded onto the column, the proteins are refolded by a linear gradient (0-0.5M NaCl) using 5 C.V. (column volumes) of a buffer that lacks the denaturant (50 mM Tris-HCl pH8.0, 10 mM 2-mercaptoethanol, plus 2 mM CaCl₂). The proteins are eluted with a buffer containing 0.5M NaCl, 50 mM Tris-HCl pH8.0, and 250 mM imidazole. The fusion tags (6× His, CII6His) are cleaved with Factor Xa or Granzyme B, and removed from protein samples by passage through a Ni⁺-NTA affinity column. The proteins are further purified by ion-exchange chromatography on Q-sepharose (GE) using linear gradients (0-0.5M NaCl) over 10 C.V. in a buffer (50 mM Tris-HCl, pH8.0 and 2 mM CaCl₂). Proteins are dialyzed into 1×PBS buffer. Optionally, endotoxin is removed by passing through a Mustang E filter (PALL).

To prepare soluble extracts from bacterial cells for expressed proteins in the periplasm, the bacterial pellets are suspended in a loading buffer (10 mM phosphate buffer pH6.0), and lysed using sonication (or alternatively a French press). After spinning down the insoluble portion in a centrifuge, the soluble extract is applied to an SP FF column (GE). Periplasmic extracts are also prepared by osmotic shock or “soft” sonication. Secreted soluble 6× His tagged proteins are purified by Ni⁺-NTA column as described above. Crude extracts are buffer exchanged into an affinity column loading buffer, and then applied to an SP FF column. After washing with 4 C.V. of loading buffer, the proteins are eluted using a 100% gradient over 8 C.V. with a high salt buffer (10 mM phosphate buffer, 0.5M NaCl, pH6.0). Eluate is filtered by passing through a Mustang E filter to remove endotoxin. The partially purified proteins are buffer exchanged into 10 mM phosphate buffer, pH7.4, and then loaded to a Q FF column. After washing with 7 C.V. with 10 mM phosphate buffer pH 6.0, the proteins are eluted using a 100% gradient over 8 C.V. with a high salt buffer (10 mM phosphate buffer, pH6.0, 0.5M NaCl). Once again endotoxin is removed by passing through a Mustang E filter.

### Example 16

Expression and Purification of HSP70 Binding Polypeptides from Mammalian Cells

Plasmids for each expression construct are prepared using a Qiagen Endofree Maxi Prep Kit. Plasmids are used to transiently transfect HEK293-EBNA cells. Tissue culture supernatants are collected for protein purification 2-4 days after transfection.

For large-scale production, stable cell lines in CHO or PER.C6 cells are developed to overexpress HSP70 binding polypeptides. Cells (5×10⁸) are inoculated into 2.5 L of media in a 20 L bioreactor (Wave). Once the cells have doubled, fresh media (1× start volume) is added, and continues to be added as cells double until the final volume reaches 10 L. The cells are cultured for about 10 days until cell viability drops to 20%. The cell culture supernatant is then collected for purification.

Both His-tagged protein purification (by Ni⁺-NTA column) and non-tagged protein purification (by ion exchange chromatography) are employed as detailed above.

### Example 17

Affinity Maturation of HSP70 Binding Polypeptides Assisted by in Silico Modeling

In silico modeling is used to affinity mature HSP70 binding polypeptides that are identified from the CTLD phage display library screening. Agonist homology models are built based on the known tetranectin 3D structures. Loop conformations of homology models of binding polypeptides are refined and optimized using LOOPER (DS2.1, Accehys) and their related algorithms. This process includes three basic steps: 1. Construction of a set of possible loop conformers with optimized interactions of loop backbone with the rest of the protein; 2. Building and structural optimization of loop side chains and energy minimization applied to all loop atoms; 3. Final scoring and ranking the retained variants of loop conformers. Potential binding regions or epitopes located HSP70 are identified for the binding polypeptides using a combination of manual and molecular dynamics-based docking. The binding domains are further confirmed by performing binding assays using deletion or point mutations of HSP70 and the binding polypeptides. Amino acid residues (or sequences) that are involved in determining binding specificity are defined on both HSP70 and HSP70 CTLD binding polypeptides. A combination of random mutations at various target positions is screened using structure-based computation to determine the compatibility with the structure template. Based on the analysis of apparent packing defects, residues are selected for mutagenesis to construct a library for phage display.

The 3D models of HSP70 agonist peptides and HSP70 can be used as a reference to refine the peptide-grafted CTLD and HSP70 modeling. When HSP70 agonist peptides are grafted into CTLD loops, loop conformations are optimized and re-surfaced to match agonist peptides/HSP70 binding by changing the flanking and surrounding amino acid residues using in silico modeling. Peptide grafted CTLD agonist homology models are built based on the known tetranectin 3D structures. Loop conformations of homology models of binding polypeptides are refined and optimized using LOOPER (DS2.1, Accelrys) and their related algorithms as described above. A combination of random mutations at various target positions is screened by structure-based computation for their compatibility with the structure template. Based on analysis of apparent packing defects, amino acid residues flanking and surrounding peptides are selected for mutagenesis to construct a library for phage display.

### Example 18

Inhibition of Cancer Cell Proliferation

Human cancer cell lines expressing HSP70 such as WM793B (melanoma) (purchased from American Type Tissue Collection (Manassas, Va.)) are cultured under the appropriate condition for each cell line and seeded at cell densities of 5,000-20,000 cells/well (as determined appropriate by growth curve for each cancer cell line). HSP70 agonistic molecules are added at concentrations ranging from 0.0001-100 μg/mL. Optionally HSP70 binding polypeptides are combined with therapeutic methods, including chemotherapeutics (e.g., bortezomib) or cells that are pre-sensitized by radiation, to generate a synergistic effect that upregulates HSP70 or alters caspase activity. The number of viable cells is assessed after 24 and 48 h using “CellTiter 96® AQᵤₑₒᵤₛ One Solution Cell Proliferation Assay” (Promega) according to the manufacturer's instructions, and the IC₅₀ concentrations for the HSP70 binding polypeptides are determined.

### Example 19

Agonist Molecule Assessment in Tumor Xenograft Models

Cancer cell lines (e.g. WM793B) are injected s.c into Balb/c nude or SCID mice. Tumor length and width is measured twice a week using a caliper. Once the tumor reaches 250 mm³ in size, mice will be randomized and treated i.v. or s.c. with 10-100 mg/kg HSP70 agonist. Treatment can be combined with other therapeutics such as chemotherapeutics (e.g. irinotecan, bortezomib, or 5FU) or radiation treatment. Tumor size is observed for 30 days unless tumor size reaches 1500 mm³ in which case mice have to be sacrificed.

### Example 20

Panning of Human Library 1-4 on Human HSP70

1. Panning on HSP70

Panning can be performed using the human Loop1-4 library of human CTLDs on HSP70/Fc antigen-coated (R&D Systems) wells prepared fresh the night before bound with 250 ng to 1 μg of the carrier free target antigen diluted in 100 μL of PBS per well. Antigen plates are incubated overnight at 4° C. then for 1 hour at 37° C., washed twice with PBS/0.05% Tween 20 and twice with PBS, and then blocked with 1% BSA/PBS for 1 hr at 37° C. prior to panning. Six wells are used in each round, and phage are bound to wells for two hours at 37° C. using undiluted, 1:10, and 1:100 dilutions in duplicates of the purified phage supernatant stock. Since target antigens are expressed as Fc fusion proteins, phage supernatant stocks contained 1 μg/mL soluble IgG1 Fc acting as soluble competitor. In addition, prior to target antigen binding, phage supernatants are pre-bound to antigen wells with human IgG1 Fc to remove Fc binders (no soluble IgG1 Fc competitor should be present during the pre-binding).

To produce phage for the initial round of panning, 10 μg of library DNA is transformed into electrocompetent TG-1 bacteria and grown in a 100 mL culture containing SB with 40 μg/mL carbenicillin and 2% glucose for 1 hour at 37° C. The carbenicillin concentration is then increased to 50 μg/mL and the culture was grown for an additional hour. The culture volume is then increased to 500 mL, and the culture is infected with helper phage at a multiplicity of infection (MOI) of 5×10⁹ pfu/mL and grown for an additional hour at 37° C. The bacteria are spun down and resuspended in 500 mL SB containing 50 μg/mL carbenicillin and 100 μg/mL kanamycin and grown overnight at room temperature shaking at 250 rpm. The following day bacteria are spun out and the phage precipitated with a final concentration of 4% PEG/0.5 M NaCl on ice for 1 hr. Precipitated phage are then spun down at 10,500 rpm for 20 minutes at 4° C. Phage pellets are resuspended in 1% BSA/PBS containing the Roche EDTA free complete protease inhibitors. Resuspended phage are then spun in a microfuge for 10 minutes at 13,200 rpm and passed through a 0.2 μM filter to remove residual bacteria.

50 μL of the purified phage supernatant stock per well are pre bound to the IgG Fc coated wells for 1 hr at 37° C. and then transferred to the target antigen coated well at the appropriate dilution for 2 hrs at 37° C. as described above. Wells are then washed with PBS/0.05% Tween 20 for 5 minutes pipeting up and down (1 wash at round 1, 5 washes at round 2, and 10 washes at rounds 3 and 4). Target antigen bound phage are eluted with 60 μL per well acid elution buffer (glycine pH 2) and then neutralized with 2M Tris 3.6 μL/well. Eluted phage are then used to infect TG-1 bacteria (2 mL at OD₆₀₀ of 0.8-1.0) for 15 minutes at room temperature. The culture volume is brought up to 10 mL in SB with 40 μg/mL carbenicillin and 2% glucose and grown for 1 hour at 37° C. shaking at 250 rpm. The carbenicillin concentration is then increased to 50 μg/mL and the culture is grown for an additional hour. The culture volume is then increased to 100 mL, and the culture is infected with helper phage at an MOI of 5×10⁹ pfu/mL and grown for an additional hour at 37° C. The bacteria are spun down and resuspended in 100 mL SB containing 50 μg/mL carbenicillin and 100 μg/mL kanamycin and grown overnight at room temperature with shaking at 250 rpm. Subsequent rounds of panning are performed similarly adjusting for smaller culture volumes, and with increased washing in later rounds. Clones are panned on HSP70/Fc for four rounds and clones obtained from screening rounds three and four.

2. Phage ELISA

Panning can be performed using the TG-1 strain of bacteria for at least four rounds. At each round of panning sample titers are taken and plated on LB plates containing 50 μg/mL carbenicillin and 2% glucose. To screen for specific binding of phagemid clones to the receptor target, individual colonies are picked from these titer plates from the later rounds of panning and grown up overnight at room temperature with shaking at 250 rpm in 250 μL of 2×YT medium containing 2% glucose and 50 μg/mL carbenicillin in a polypropylene 96-well plate with an air-permeable membrane on top. The following day a replica plate is set up in a 96-deep-well plate by inoculating 500 μL of 2×YT containing 2% glucose and 50 μg/mL carbenicillin with 30 μL of the previous overnight culture. The remaining overnight culture is used to make a master stock plate by adding 100 μL of 50% glycerol to each well and storing at −80° C. The replica culture plate is grown at 37° C. with shaking at 250 rpm for approximately 2 hrs until the OD₆₀₀ is 0.5-0.7. The wells are then infected with K07 helper phage to 5×10⁹ pfu/mL mixed and incubated at 37° C. for 30 minutes without shaking, then incubated an addition 30 minutes at 37° C. with shaking at 250 rpm. The cultures are then spun down at 2500 rpm and 4° C. for 20 minutes. The supernatants are removed from the wells and the bacterial cell pellets are re-suspended in 500 μL of 2×YT containing 50 μg/mL carbenicillin and 50 μg/mL kanamycin. An air-permeable membrane is placed on the culture block and cells are grown overnight at room temperature with shaking at 250 rpm.

On day 3, cultures are spun down and supernatants containing the phage are blocked with 3% milk/PBS for 1 hr at room temperature. An initial Phage ELISA is performed using 75-100 ng of antigen bound per well. Non-specific binding is measured using 75-100 ng of human IgG1 Fc per well. HSP70/Fc antigen (R&D Systems)-coated wells and IgG Fc coated wells are prepared fresh the night before by binding the above amount of antigen diluted in 100 μL of PBS per well. Antigen plates are incubated overnight at 4° C. then for 1 hour at 37° C., washed twice with PBS/0.05% Tween 20 and twice with PBS, and then blocked with 3% milk/PBS for 1 hr at 37° C. prior to the ELISA. Blocked phage are bound to blocked antigen-bound plates for 1 hr then washed twice with 0.05% Tween 20/PBS and then twice more with PBS. A HRP-conjugated anti-M13 secondary antibody diluted in 3% milk/PBS is then applied, with binding for 1 hr and washing as described above. The ELISA signal is developed using 90 μL TMB substrate mix and then stopped with 90 μL 0.2 M sulfuric acid, then ELISA plates are read at 450 nM.

### Example 21

Subcloning and Production of ATRIMER™ binders to Human HSP70

The loop region DNA fragments can be released from HSP70 binder DNA by double digestion with BglII and MfeI restriction enzymes, and are ligated to bacterial expression vectors pANA4, pANA10 or pANA19 to produce secreted ATRIMER™ in E. coli.

The expression constructs are transformed into E. coli strains BL21 (DE3), and the bacteria are plated on LB agar with ampicillin. Single colony on a fresh plate is inoculated into 2×YT medium with ampicillin. The cultures are incubated at 37° C. in a shaker at 200 rpm until OD600 reached 0.5, then cooled to room temperature. Arabinosis is added to a final concentration of 0.002-0.02%. The induction is performed overnight at room temperature with shaking at 120-150 rpm, after which the bacteria are collected by centrifugation. The periplasmic proteins are extracted by osmotic shock or gentle sonication.

The 6× His-tagged ATRIMERs™ are purified by Ni⁺-NTA affinity chromatography. Briefly, periplasmic proteins are reconstituted in a His-binding buffer (100 mM HEPES, pH 8.0, 500 mM NaCl, 10 mM imidazole) and loaded onto a Ni⁺-NTA column pre-equivalent with His-binding buffer. The column is washed with 10× vol. of binding buffer. The proteins are eluted with an elution buffer (100 mM HEPES, pH 8.0, 500 mM NaCl, 500 mM imidazole). The purified proteins are dialyzed into PBS buffer and bacterial endotoxin is removed by anion exchange.

The strep II-tagged ATRIMERs™ are purified by Strep-Tactin affinity chromatography. Briefly, periplasmic proteins are reconstituted in 1× binding buffer (20 mM Tris-HCl, pH 8.5, 150 mM NaCl, 2 mM CaCl₂, 0.1% Triton X-100) and loaded onto a Strep-Tactin column pre-equivalent with binding buffer. The column is washed with 10× vol. of binding buffer. The proteins are eluted with an elution buffer (binding buffer with 2.5 mM desthiobiotin). The purified proteins are dialyzed into binding buffer and bacterial endotoxin is removed by anion exchange.

The DNA fragments of loop region are sub-cloned into mammalian expression vectors pANA2 and pANA11 to produce ATRIMERs™ in a HEK293 transient expression system. The DNA fragments of the loop region are released from IL-23R binder DNA by double digestion with BglII and MfeI restriction enzymes, and ligated to the expression vectors pANA2 and pANA11, which are pre-digested with BglII and MfeI. The expression plasmids are purified from bacteria by Qiagen HiSpeed Plasmid Maxi Kit (Qiagene). For HEK293 adhesion cells, the transient transfection is performed by Qiagen SuperFect Reagent (Qiagene) according to the manufacturer's protocol. The day after transfection, the medium is removed and changed to 293 Isopro serum-free medium (Irvine Scientific). Two days later, 20% glucose in 0.5M HEPES is added into the media to a final concentration of 1%. The tissue culture supernatant is collected 4-7 days after transfection for purification. For HEK293F suspension cells, the transient transfection is performed by Invitrogen's 293Fectin and its protocol. The next day, 1× volume of fresh medium is added into the culture. The tissue culture supernatant is collected 4-7 days after transfection for purification. The His- or Strep II-tagged ATRIMER™ purification from mammalian tissue culture supernatant is performed as described above.

The DNA fragments of loop region are sub-cloned into mammalian expression vectors pANA5, pANA6, pANA7, pANA8 and pANA9 to produce ATRIMER™atrimers complexes with different CTLD-presenting orientations in the HEK293 transient expression system. pANA5 is a modified pCEP4 vector containing a C-terminal His-tag and a V₄₉ deletion in human TN. Similarly, pANA6 has a T₄₈ deletion, and pANA7 has T₄₈ and V₄₉ deletions. pANA8 has a C₅₀,C₆₀→S₅₀,S₆₀ double mutation to provide a more flexible CTLD than wildtype TN.pANA9 has E₁-V₁₇ deletions to remove the glycosylation site. The DNA fragments of loop region are released from HSP70 binder DNA by double digestion with BglII and MfeI restriction enzymes, and are ligated to the expression vectors pANA5, pANA6, pANA7, pANA8 and pANA9, which are pre-digested with BglII and MfeI.

### Example 22

Characterization of the Affinity of Human HSP70 Binders using Biacore

Immobilization of an anti-human IgG Fc antibody (Biacore) to the CM5 chip (Biacore) is performed using standard amine coupling chemistry and this surface is used to capture recombinant human HSP70 Fc fusion protein (R&D Systems). ATRIMER™ COMPLEX dilutions (1-500 nM) are injected over the HSP70 surface at 30 μl/min and kinetic constants are derived from the sensorgram data using the Biaevaluation software (version 3.1, Biacore). Data collection is 3 minutes for the association and 5 minutes for dissociation. The anti-human IgG surface is regenerated with a 30 s pulse of 3 M magnesium chloride. All sensorgrams are double-referenced against an activated and blocked flow-cell as well as buffer injections.

### Example 23

Panning of NEB Peptide Libraries on Human HSP70 and Identification of a HSP70 Specific Peptide

Panning of peptide libraries can be performed using the New England Biolabs (NEB) Phage Display Libraries. Panning is performed on HSP70/Fc antigen-coated (R&D Systems) wells prepared fresh the night before bound with 3 μg of the carrier free target antigen diluted in 150 μL of 0.1M NaHCO₃ pH 8.6 per well. Duplicate wells are used in each round. Antigen plates are incubated overnight at 4° C. then for 1 hour at 37° C. The antigen is removed and the well is then blocked with 0.5% boiled Casein in PBS pH 7.4 for 1 hr at 37° C. prior to panning. The Casein is then removed and wells are then washed 6× with 300 μL of TBST (0.1% Tween), then phage are added. Since target antigens are expressed as Fc fusion proteins, prior to target antigen binding, phage supernatants are pre-bound for 1 hr to antigen wells with human IgG1 Fc to remove Fc binders (during rounds 2 through 4). Fc antigen bound wells are prepared similar to HSP70/Fc antigen bound wells as detailed above.

For the initial round of panning, 100 μL of TBST(0.1% Tween) is added to each well and 5 ul of each of the 3 NEB peptide libraries (Ph.D.-7, Ph.D.-12, and Ph.D.-C7C) are added to each well. The plate is rocked gently for 1 hr at room temperature, then washed 10× with TBST(0.1% Tween). Bound phage are eluted with 100 μL of PBS containing soluble DR5/Fc target antigen at a concentration of 100 μg/ml. Phage are eluted for 1 hr rocking at room temperature. Eluted phage are then removed from the wells and used to infect 20 mls of ER2738 bacteria at an OD_{600 nm} of 0.05 to 0.1, and grown shaking at 250 rpm at 37° C. for 4.5 hrs. Bacteria are then spun out of the culture at 12K×G for 20 min at 4° C. Bacteria are transferred to a fresh tube and re-spun. The supernatant is again transferred to a fresh tube and the Phage are precipitated by adding ⅙^{th} the volume of 20% PEG/2.5M NaCl. Phage are precipitated overnight at 4° C. The following day the precipitated phage are spun down at 12K×G for 20 min at 4° C. The supernatant is discarded and the phage pellet re-suspended in 1 ml of TBST(0.1% Tween). Residual bacteria are cleared by spinning in a microfuge at 13.2K for 10 minutes at 4° C. The phage supernatant is then transferred to a new tube and re-precipitated by adding ⅙^{th} the volume of 20% PEG/2.5M NaCl, and incubating at 4° C. on ice for 1 hr. The precipitated phage are spun down in a microfuge at 13.2K for 10 minutes at 4° C. The supernatant is discarded and the phage pellet re-suspended in 200 μL of TBS. Subsequent rounds of panning are performed similar to round 1 with the exception phage are pre-bound for 1 hr to Fc coated wells and that 4 μL of the amplified phage stock from the previous round are used per well during the binding. In addition the tween concentration is increased to 0.5% in the TBST used during the 10 washes.

Phage ELISA

Panning is performed using the ER2738 strain of bacteria for at least four rounds. At each round of panning sample titers are taken and plated using top agar on LB/Xgal plates to obtain plaques. To screen for specific binding of phage clones to the receptor target, individual plaques are picked from these titer plates from the later rounds of panning and used to infect ER2738 bacteria at an OD_{600 nm} of 0.05 to 0.1, and grown shaking at 250 rpm at 37° C. for 4.5 hrs. Then stored at 4° C. overnight.

On day 2, cultures are spun down at 12K×G for 20 min at 4° C., and supernatants containing the phage are blocked with 3% milk/PBS for 1 hr at room temperature. An initial Phage ELISA is performed using 75-100 ng of DR5/Fc antigen bound per well. Non-specific binding is measured using wells containing 75-100 ng of human IgG1 Fc petr well. HSP70/Fc antigen (R&D Systems)-coated wells and IgG1 Fc coated wells are prepared fresh the night before by binding the above amount of antigen diluted in 100 μL of PBS per well. Antigen plates are incubated overnight at 4° C. then for 1 hour at 37° C., washed twice with PBS/0.05% Tween 20 and twice with PBS, and then blocked with 3% milk/PBS for 1 hr at 37° C. prior to the ELISA. Blocked phage are bound to blocked antigen-bound plates for 1 hr then washed twice with 0.05% Tween 20/PBS and then twice more with PBS. A HRP-conjugated anti-M13 secondary antibody diluted in 3% milk/PBS is then applied, with binding for 1 hr and washing as described above. The ELISA signal is developed using 90 μL TMB substrate mix and then stopped with 90 μL 0.2 M sulfuric acid, then ELISA plates are read at 450 nM.

Although various specific embodiments of the present invention have been described herein, it is to be understood that the invention is not limited to those precise embodiments and that various changes or modifications can be affected therein by one skilled in the art without departing from the scope and spirit of the invention.

The disclosures of all references and publications cited herein are expressly incorporated by reference in their entireties to the same extent as if each were incorporated by reference individually.

### BIBLIOGRAPHY

Agnew, Chem Intl. Ed. Engl., 33: 183-186 (1994)

Asea A, Kraeft S K, Kurt-Jones E A, Stevenson M A, Chen L B, Finberg R W, Koo G C, Calderwood S K. HSP70 stimulates cytokine production through a CD14-dedendant pathway, demonstrating its dual role as a chaperone and cytokine. Nature Med 6: 435-442 (2000).

Ashkenazi, et al. J Clin Invest.; 104(2):155-62 (July 1999).

Aspberg, A., Miura, R., Bourdoulous, S., Shimonaka, M., Heinegard, D., Schachner, M., Ruoslahti, E., and Yamaguchi, Y. (1997). “The C-type lectin domains of lecticans, a family of aggregating chondroitin sulfate proteoglycans, bind tenascin-R by protein-protein interactions independent of carbohydrate moiety”. Proc. Natl. Acad. Sci. (USA) 94: 10116-10121

Ausubel et al., Current Protocols in Molecular Biology (eds., Green Publishers Inc. and Wiley and Sons 1994

Bass, S., Greene, R., and Wells, J. A. (1990). “Hormone phage: an enrichment method for variant proteins with altered binding properties”. Proteins 8: 309-314

Basu S, Binder R J, Ramalingam T, Srivastava P K. CD91 is a common receptor for heat shock proteis gp96, hsp90, hsp70, and calreticulin Immunity 14: 303-313 (2001).

Becker T, Hartl F U, Wieland F. CD40, an extracellular receptor for binding and uptake of HSP70-peptide complexes. J Cell Biol 158: 1277-1285 (2002).

Benhar, I., Azriel, R., Nahary, L., Shaky, S., Berdichevsky, Y., Tamarkin, A, and Wels, W. “Highlyefficient selection of phage antibodies mediated by display of antigen as Lpp-OmpA' fusions on live bacteria” J. Mol. Biol. 301: 893-904 (2000).

Benhar, I., Azriel, R., Nahary, L., Shaky, S., Berdichevsky, Y., Tamarkin, A., and Weis, W. (2000). “Highly efficient selection of phage antibodies mediated by display of antigen as Lpp-OmpA' fusions on live bacteria”. J. Mol. Biol. 301: 893-904

Berglund, L. and Petersen, T. E. (1992). “The gene structure of tetranectin, a plasminogen binding protein”. FEBS Letters 309: 15-19

Bertrand, J. A., Pignol, D., Bernard, J-P., Verdier, J-M., Dagorn, J-C., and Fontecilla-Camps, J. C. (1996). “Crystal structure of human lithostathine, the pancreatic inhibitor of stone formation”. EMBO J. 15: 2678-2684

Berwyn B, Hart J P, Rice S, Gass C, Pizzo S V, Post S R, Nicchitta C V. Scavenger receptor-A mediates gp96/GRP94 and calreticulin internalization by antigen-presenting cells. EMBO J 22: 6127-6136 (2003)

Bettler, B., Texido, G., Raggini, S., Ruegg, D., and Hofstetter, H. (1992). “Immunoglobulin E-binding site in Fc epsilon receptor (Fc epsilon RII/CD23) identified by homolog-scanning mutagenesis”. J. Biol. Chem. 267: 185-191

Blanck, O., Iobst, S. T., Gabel, C., and Drickamer, K. (1996). “Introduction of selectin-like binding specificity into a homologous mannose-binding protein”. J. Biol. Chem. 271: 7289-7292

Boder, E. T. and Wittrup, K. D. “Yeast surface display for screening combinatorial polypeptide libraries”. Nature Biotech. 15: 553-557 (1997).

Burrows L, Iobst S T, Drickamer K. (1997) “Selective binding of N-acetylglucosamine to the chicken hepatic lectin”. Bio-chem J. 324:673-680

Castelli C, Rivoltini L, Rini F, Belli F, Testori A, Maio M, Mazzaferro V, Coppra Srivastava P K. Heat shock proteins: biological functions and clinical application as personalized vaccines for human cancer. Cancer Immunol Immunother 53:227-33 (2004)

Cha et. al., J Biol Chem., 275(40):31171-7 (Oct. 6, 2000).

Chemotherapy Service Ed., M. C. Perry, Williams & Wilkins, Baltimore, Md. (1992)

Chen C H, Wang T L, Hung C F, Yang Y, Young R A, Pardoll D M, Wu, T C. Enhancement of DNA vaccine potency by linkage of antigen gene to an HSP70 gene. Cancer Res 60: 1035-1042 (2000)

Chiba, H., Sano, H., Saitoh, M., Sohma, H., Voelker, D. R., Akino, T., and Kuroki, Y. (1999). “Introduction of mannose binding protein-type phosphatidylinositol recognition into pulmonary surfactant protein A”. Biochemistry 38: 7321-7331

Christensen, J. H., Hansen, P. K., Lillelund, O., and Thogersen, H. C. (1991). “Sequence-specific binding of the N-terminal three-finger fragment of Xenopus transcription factor IIIA to the internal control region of a 5S RNA gene”. FEBS Letters 281: 181-184

Cyr, J. L. and Hudspeth, A. J. (2000). “A library of bacteriophage-displayed antibody fragments directed against proteins of the inner ear”. Proc. Natl. Acad. Sci (USA) 97: 2276-2281

Das P K, van den Wijngaard R M J G J, Wankowicz-Kaoinska A, Le Poole I C. A symbiotic concept of autoimmunity and tumor immunity: lessons from vitiligo. Trends Immunol 22: 130-136 (2001)

Degli-Esposti et al., Immunity, 7(6):813-820 (December 1997)

Degli-Esposti et al., J. Exp. Med., 186(7):1165-1170 (Oct. 6, 1997)

Delneste Y, Magistrelli G, Gauchat J, Haeuw J, Aubry J, Nakamura K, Kawakami-Honda N, Goetsch L, Sawamura T, Bonnefoy J, Jeannin P. Involvement of LOX-1 in dendritic cell-mediated antigen cross-presentation. Immunity 17: 353-362 (2002)

Drickamer, K. (1992). “Engineering galactose-binding activity into a C-type mannose-binding protein”. Nature 360: 183-186

Drickamer, K. (1999). “C-type lectin-like domains”. Curr. Opinion Struc. Biol. 9: 585-590

Drickamer, K. and Taylor, M. E. (1993). “Biology of animal lectins”. Annu. Rev. Cell Biol. 9: 237-264

Dunn, I. S. “Phage display of proteins”. Cum Opinion Biotech. 7: 547-553 (1996).

Dunn, I. S. (1996). “Phage display of proteins”. Curr. Opinion Biotech. 7: 547-553

Engelhorn M E, Guevara-Patino J A, Noffz G, Hooper A T, Lou O, Gold J S, Kappel B J, Houghton A N. Autoimmunity and tumor immunity induced by immune responses to mutations in self. Nat Med 12: 198-206 (2006)

Erbe, D. V., Lasky, L. A., and Presta, L. G. “Selectin variants”. U.S. Pat. No. 5,593,882

Ernst, W. J., Spenger, A, Toellner, L., Katinger, H., Grabhen; R. M. “Expanding baculovirus surface display. Modification of the native coat protein gp64 of Autographa calfornica NPV”. Eur. J. Biochem. 267:4033-4039 (2000).

Ewart, K. V., Li, Z., Yang, D. S. C., Fletcher, G. L., and Hew, C. L. (1998). “The ice-binding site of Atlantic herring antifreeze protein corresponds to the carbohydrate-binding site of C-type lectins”. Biochemistry 37: 4080-4085

Feinberg, H., Park-Snyder, S., Kolatkar, A. R., Heise, C. T., Taylor, M. E., and Weis, W. I. (2000). “Structure of a C-type carbohydrate recognition domain from the macrophage mannose receptor”. J. Biol. Chem. 275: 21539-21548

Frediani T, Lucarelli S, Frediani S, Frati C. The role of heat shock proteins in vitiligo: deviation of cytotoxic response. J Dermatol Sci 37: 114-117 (2005)

Fujii, I., Fukuyama, S., Iwabuchi, Y., and Tanimura, R. (1998). “Evolving catalytic antibodies in a phage-displayed combinatorial library”. Nature Biotech. 16: 463-467

Garbelli S, Mantovani S, Palermo B, Giachini C. Melanocyte-specific, cytotoxic T cell responses in vitiligo: the effective variant of melanoma immunity. Pigment Cell Res 18: 234-242 (2005)

Gates, C. M., Stemmer, W. P. C., Kaptein, R., and Schatz, P. J. “Affinity selective isolation of ligands from peptide libraries through display on a lac repressor “headpiece dimer”. J. Mol. Biol. 255: 373-386 (1996).

Gething M J, Sambrook J., Protein folding in the cell. Nature 355: 33-45 (1992).

Graversen, J. H., Jacobsen, C., Sigurskjold, B. W., Lorentsen, R. H., Moestrup, S. K., Thogersen, H. C., and Etzerodt, M. (2000). “Mutational Analysis of Affinity and Selectivity of Kringle-Tetranectin Interaction. Grafting novel kringle affinity onto the tetranectin lectin scaffold”. J. Biol. Chem. 275: 37390-37396

Graversen, J. H., Lorentsen, R. H., Jacobsen, C., Moestrup, S. K., Sigurskjold, B. W., Thogersen, H. C., and Etzerodt, M. (1998). “The plasminogen binding site of the C-type lectin tetranectin is located in the carbohydrate recognition domain, and binding is sensitive to both calcium and lysine”. J. Biol. Chem. 273:29241-29246

Griffiths, A. D. and Duncan, A. R. “Strategies for selection of antibodies by phage display”. Curr. Opinion Biotech. 9: 102-108 (1998).

Gross C, Hansch D, Gastpar R, Multhoff G. Interaction of heat shock protein 70 peptide with NK cells involves the NK receptor CD94. Biol Chem 384: 267-279 (2003)

Hakansson, K., Lim, N. K., Hoppe, H-J., and Reid, K. B. M. (1999). “Crystal structure of the trimeric alpha-helical coiled-coil and the three lectin domains of human lung surfactant protein D”. Structure Folding and Design 7: 255-264

Holtet, T. L., Graversen, J. H., Clemmensen, I., Thogersen, H. C., and Etzerodt, M. (1997). “Tetranectin, a trimeric plasminogen-binding C-type lectin”. Prot. Sci. 6: 1511-1515

Honma, T., Kuroki, Y., Tzunezawa, W., Ogasawara, Y., Sohma, H., Voelker, D. R., and Akino, T. (1997). “The mannose-binding protein A region of glutamic acid185-alanine221 can functionally replace the surfactant protein A region of glutamic acid195-phenylalanine228 without loss of interaction with lipids and alveolar type II cells”. Biochemistry 36: 7176-7184

Houghton A N, Guevara-Patino J A. Immune recognition of self in immunity against cancer. J Clin Invest 114: 468-471 (2004)

Huang, W., Zhang, Z., and Palzkill, T. (2000). “Design of potent beta-lactamase inhibitors by phage display of beta-lactamase inhibitory protein”. J. Biol. Chem. 275: 14964-14968

Hufton, S. E., van Neer, N., van den Beuken, T., Desmet, J., Sablon, E., and Hoogenboom, H. R. (2000). “Development and application of cytotoxic T lymphocyte-associated antigen 4 as a protein scaffold for the generation of novel binding ligands”. FEBS Letters 475: 225-231

Iobst, S. T. and Drickamer, K. (1994). “Binding of sugar ligands to Ca(2+)-dependent animal lectins. II. Generation of high-affinity galactose binding by site-directed mutagenesis”. J. Biol. Chem. 269: 15512-15519

Iobst, S. T. and Drickamer, K. (1996). “Selective sugar binding to the carbohydrate recognition domains of the rat hepatic and macrophage asialoglycoprotein receptors”. J. Biol. Chem. 271: 6686-6693

Iobst, S. T., Wormald, M. R., Weis, W. I., Dwek, R. A., and Drickamer, K. (1994). “Binding of sugar ligands to Ca(2+)-dependent animal lectins. I. Analysis of mannose binding by site-directed mutagenesis and NMR”. J. Biol. Chem. 269: 15505-15511

Janeway, Nature, 341(6242): 482-3 (Oct. 12, 1989)

Jaquinod, M., Holtet, T. L., Etzerodt, M., Clemmensen, I., Thogersen, H. C., and Roepstorff, P. (1999). “Mass Spectrometric Characterisation of Post-Translational Modification and Genetic Variation in Human Tetranectin”. Biol. Chem. 380: 1307-1314

Jin et al, Cancer Res., 15;64(14):4900-5 (July 2004).

Johnson J D, Fleshner M. Releasing signals, secretory pathways, and immune function of endogenous extracellular heat shock protein 72. J Leukoc Biol 79: 425-434 (2006)

Kammerer R, Stober D, Riedl P, Oehninger C, Schirmbeck R, Reimann J. Noncovalent association with stress protein facilitates cross-priming of CD8+ T cells to tumor cell antigens by dendritic cells. J Immunol 168: 108-117 (2002)

Kastrup, J. S., Nielsen, B. B., Rasmussen, H., Holtet, T. L., Graversen, J. H., Etzerodt, M., Thogersen, H. C., and Larsen, I. K. (1998). “Structure of the C-type lectin carbohydrate recognition domain of human tetranectin”. Acta. Cryst. D 54: 757-766

Kiang J G, Tsokos G C. Heat shock protein 70 kDa: molecular biology, biochmeisty and physiology. Pharmacol Ther 80: 183-201 (1998)

Kogan, T. P., Revelle, B. M., Tapp, S., Scott, D., and Beck, P. J. (1995). “A single amino acid residue can determine the ligand specificity of E-selectin”. J. Biol. Chem. 270: 14047-14055

Kolatkar, A. R., Leung, A. K., Isecke, R., Brossmer, R., Drickamer, K., and Weis, W. I. (1998). “Mechanism of N-acetylgalactosamine binding to a C-type animal lectin carbohydrate-recognition domain”. J. Biol. Chem. 273: 19502-19508

Langer et al., J. Biomed. Mater. Res., 15: 167-277 (1981)

Langer, Chem. Tech., 12: 98-105 (1982)

Le Poole I C, Boissy R E, Sarangarajan R, Chen J, Forristal J J, Sheth P, Westerhof W, Babcock G, Das P K, Saelinger CB PIG3V, an immortalized human vitiligo melanocyte cell line, expresses dilated endoplasmic reticulum. In Vitro Cell Dev Biol Anim.36 :309-319. (2000)

Le Poole I C, Stennett L S, Bonsih B K, Robinson J K, Hernandez C, Hann S K, Nickoloff B J. Expansion of vitiligo lesions is associated with reduced epidermal CDw60 expression and increased expression of HLA-DR in perilesional skin. Br J Dermatol 149: 739-748 (2003)

Le Poole I C, van den Wijngaard R M J G J, Westerhof W, Dutrieux R P, Das P K. Presence or absence of melanocytes in vitiligo lesions: An immunohistochemical investigation. J Invest Dermatol 100: 816-822 (1993)

Le Poole I C, van den Wijngaard R M J G J, Westerhof W, Verkruisen R P, Dutrieux R P, Dingemans K P, Das P K. Phagocytosis by normal human melanocytes in vitro. Exp Cell Res 205: 388-395 (1993)

Le Poole I C, Wankowicz-Kalinska A, van den Wijngaard R M J G J, Nickoloff B J, Das P K. Autoimmune aspects of depigmentation in vitiligo. J Investig Dermatol Symp Proc. 2004 January; 9(1):68-72. (2004)

Lehner T, Wang Y, Whittal T, McGowan E, Kelly C G, Singh M. Functional domains of HSP70 stimulate generation of cytokines and chemokines, maturation of dendritic cells and adjuvanticity. Biochem Soc Trans 32: 629-632 (2004)

Lorentsen, R. H., Graversen, 3. H., Caterer, N. R., Thogersen, H. C., and Etzerodt, M. (2000). “The heparin-binding site in tetranectin is located in the N-terminal region and binding does not involve the carbohydrate recognition domain”. Biochem. J. 347: 83-87

MacAry P A, Javid B, Floto R A, Smith K G C, Oehlmann W, Singh M, Lehner P J. HSP70 peptide binding mutants separate antigen delivery from dendritic cell stimulation. Immunity 20: 95-106 (2004)

Mann K, Weiss I M, Andre S, Gabius H J, Fritz M. (2000). “The amino-acid sequence of the abalone (Haliotis laevigata) nacre protein perlucin. Detection of a functional C-type lectin domain with galactose/mannose specificity”. Eur. J. Biochem. 267: 5257-5264

Marks, J. D., Hoogenboom, H. R., Griffiths, A. D., and Winter, G. “Molecular evolution of proteins on filamentous phage. Mimicking the strategy of the immune system”. J. Biol. Chem. 267: 16007-16010 (1992).

Marsters et al., Curr. Biol., 7:1003-1006 (1997)

McCafferty, J., Jackson, R. H., and Chiswell, D. J. (1991). “Phage-enzymes: expression and affinity chromatography of functional alkaline phosphatase on the surface of bacterio-phage”. Prot. Eng. 4: 955-961

McCormack, F. X., Festa, A. L., Andrews, R. P., Linke, M., and Walzer, P. D. (1997). “The carbohydrate recognition domain of surfactant protein A mediates binding to the major surface glycoprotein of Pneumocystis carinii”. Biochemistry 36: 8092-8099

McCormack, F. X., Kuroki, Y., Stewart, J. J., Mason, R. J., and Voelker, D. R. (1994). “Surfactant protein A amino acids Glu195 and Arg197 are essential for receptor binding, phospholipid aggregation, regulation of secretion, and the facilitated uptake of phospholipid by type II cells”. J. Biol. Chem. 269: 29801-29807

McFarlane et al., J. Biol. Chem., 272:25417-25420 (1997)

Meier, M., Bider, M. D., Malashkevich, V. N., Spiess, M., and Burkhard, P. (2000). “Crystal structure of the carbohydrate recognition domain of the Hi subunit of the asialoglycoprotein receptor”. J. Mol. Biol. 300: 857-865

Mikawa, Y. G., Maruyama, I. N., and Brenner, S. “Surface display of proteins on bacteriophage lambda heads”. J. Mol. Biol. 262: 21-30 (1996).

Mio H, Kagami N, Yokokawa S, Kawai H, Nakagawa S, Takeuchi K, Sekine S, Hiraoka A. (1998). “Isolation and characterization of a cDNA for human mouse, and rat full-length stem cell growth factor, a new member of C-type lectin superfamily”. Biochem. Biophys. Res. Commun. 249: 124-130

Mizuno, H., Fujimoto, Z., Koizumi, M., Kano, H., Atoda, H., and Morita, T. (1997). “Structure of coagulation factors IX/X-binding protein, a heterodimer of C-type lectin domains”. Nat. Struc. Biol. 4: 438-441 [0287] Ng, K. K., Park-Snyder, S., and Weis, W. I. (1998a). “Ca.sup.2+-dependent structural changes in C-type mannose-binding proteins”. Biochemistry 37: 17965-17976

Mongkolsapaya et al., J. Immunol., 160:3-6 (1998)

Mordenti et al., Pharmaceut. Res., 8:1351 (1991)

Multhoff G, Mizzen L, Winchester C C, Milner C M, Wenk S, Eissner G, Kampinga H H, Laumbacher B, Johnson J. Heat shock protein 70 (Hsp70) stimulates proliferation and cytolytic activity of natural killer cells. Experimental hematology 27: 1627-1636 (1999).

Murakami et al., The Molecular Basis of Cancer, Mendelsohn and Israel, eds., Chapter 1, entitled “Cell cycle regulation, oncogenes, and antineoplastic drugs” by (WB Saunders: Philadelphia, pg. 13 (1995).

Neame, et al,. Protein Sci., 1(1):161-8 (1992)

Neame, P. J. and Boynton, R. E., Protein Soc. Symposium, (Meeting date 1995; 9th Meeting: Tech. Prot. Chem VII). Proceedings pp. 401-407 (Ed., Marshak, D. R.; Publisher: Academic, San Diego, Calif.) (1996).

Ng, K. K. and Weis, W. I. (1998b). “Coupling of prolyl peptide bond isomerization and Ca2+ binding in a C-type mannose-binding protein”. Biochemistry 37: 17977-17989

Nielsen, B. B., Kastrup, J. S., Rasmussen, H., Holtet, T. L., Graversen, J. H., Etzerodt, M., Thogersen, H. C., and Larsen, I. K. (1997). “Crystal structure of tetranectin, a trimeric plasminogen-binding protein with an alpha-helical coiled coil”. FEBS Letters 412: 388-396

Nissim A., Hoogenboom, H. R., Tomlinson, I. M., Flynn, G., Midgley, C., Lane, D., and Winter, G. (1994). “Antibody fragments from a ‘single pot’ phage display library as immunochemical reagents”. EMBO J. 13: 692-698

Nylandsted J, Gyrd-Hansen M, Danielewicz A, Fehrenbacher N, Lademann U, Hoyer Hansen M, Weber E, Multhoff G, Rohde M, Jaatela M. Heat shock protein 70 promotes cell survival by inhibiting lysosomal membrane permeabilization. J Exp Med 200: 425-435 (2004)

Offner et al., Science, 251: 430-432 (1991)

Ogasawara, Y. and Voelker, D. R. (1995). “Altered carbohydrate recognition specificity engineered into surfactant protein D reveals different binding mechanisms for phosphatidylinositol and glucosylceramide”. J. Biol. Chem. 270: 14725-14732

Ohtani, K., Suzuki, Y., Eda, S., Takao, K., Kase, T., Yamazaki, H., Shimada, T., Keshi, H., Sakai, Y., Fukuoh, A., Sakamoto, T., and Wakamiya, N. (1999). “Molecular cloning of a novel human collectin from liver (CL-L1)”. J. Biol. Chem. 274: 13681-13689

Pan et al., FEBS Letters, 424:41-45 (1998)

Pan et al., Science, 276:111-113 (1997)

Pan et al., Science, 277:815-818 (1997)

Pattanajitvilai, S., Kuroki, Y., Tsunezawa, W., McCormack, F. X., and Voelker, D. R. (1998). “Mutational analysis of Arg197 of rat surfactant protein A. His197 creates specific lipid uptake defects”. J. Biol. Chem. 273: 5702-5707

Poget, S. F., Legge, G. B., Proctor, M. R., Butler, P. J., Bycroft, M., and Williams, R. L. (1999). “The structure of a tunicate C-type lectin from Polyandrocarpa misakiensis complexed with D-galactose”. J. Mol. Biol. 290: 867-879

Remington's Pharmaceutical Sciences, 16th edition, Osol, A. ed. (1980)

Revelle, B. M., Scott, D., Kogan, T. P., Zheng, J., and Beck, P. J. (1996). “Structure-function analysis of P-selectinsialyl LewisX binding interactions. Mutagenic alteration of ligand binding specificity”. J. Biol. Chem. 271: 4289-4297

S. G. Hymowitz, et. al., Mol Cell. 1999 October; 4(4):563-71)

Sambrook, et al. Molecular Cloning: A Laboratory Manual. Cold Spring Harbor Laboratory Press, Cold Spring Harbor, N.Y. (1989)

Sano, H., Kuroki, Y., Honma, T., Ogasawara, Y., Sohma, H., Voelker, D. R., and Akino, T. (1998). “Analysis of chimeric proteins identifies the regions in the carbohydrate recognition domains of rat lung collections that are essential for interactions with phospholipids, glycolipids, and alveolar type II cells”. J. Biol. Chem. 273: 4783-4789

Schaffitzel, C., Hanes, 3., Jermutus, L., and Plucktun, A. (1999). “Ribosome display: an in vitro method for selection and evolution of antibodies from libraries”. J. Zmmunol. Methods 23 1: 11 9-135 (1999).

Schneider et al., FEBS Letters, 416:329-334 (1997)

Screaton et al., Curr. Biol., 7:693-696 (1997)

Sheridan et al., Science, 277:818-821 (1997)

Sheriff, S., Chang, C. Y., and Ezekowitz, R. A. (1994). “Human mannose-binding protein carbohydrate recognition domain trimerizes through a triple alpha-helical coiled-coil”. Nat. Struc. Biol. 1: 789-794

Sidman et al., Biopolymers, 22: 547-556 (1983)

Sorensen, C. B., Berglund, L., and Petersen, T. E. (1995). “Cloning of a cDNA encoding murine tetranectin”. Gene 152: 243-245

Srivastava P K, Udono H. Heat shock protein-peptide complexes in cancer immunotherapy. Curr Opin Immunol 6: 728-732 (1994)

Tavaria M, Gabriele T, Kola I, Anderson R L. A hitchhiker's guide to the human Hsp70 family. Cell Stress Chap 2: 23-28 (1996)

Torgersen, D., Mullin, N. P., and Drickamer, K. (1998). “Mechanism of ligand binding to E- and P-selectin analyzed using selectin/mannose-binding protein chimeras”. J. Biol. Chem. 273: 6254-6261

Tormo, J., Natarajan, K., Margulies, D. H., and Mariuzza, R. A. (1999). “Crystal structure of a lectin-like natural killer cell receptor bound to its MHC class I ligand”. Nature 402: 623-631

Tsunezawa, W., Sano, H., Sohma, H., McCormack, F. X., Voelker, D. R., and Kuroki, Y. (1998). “Site-directed mutagenesis of surfactant protein A reveals dissociation of lipid aggregation and lipid uptake by alveolar type II cells”. Biochim. Biophys. Acta 1387: 433-446

Turk M J, Wolchok J D, Guevara-Patino J A, Goldberg S M, Houghton A N. Multiple pathways to tumor immunity and concomitant autoimmunity. Immunol Rev 188: 122-135 (2002)

Walczak et al., EMBO J., 16:5386-5387 (1997)

Wang B, Chen Y B, Ayalon O, Bender J, Garen A. Human single-chain Fv immunoconjugates targeted to a melanoma-associated chondroitin sulfate proteoglycan mediate specific lysis of human melanoma cells by natural killer cells and complement. Proc Natl Acad Sci USA. Feb. 16; 1996(4):1627-32. (1999)

Wang Y, Kelly C G, Singh M, McGowan E G, Carrara A S, Bergmeier L A, Lehner T. Stimulation of Th1-polarizing cytokines, C-C chemokines, maturation of dendritic cells and adjuvant function by the peptide binding fragment of heat shock protein 70. J Immuno 169: 2422-2429 (2002)

Wang Y, Whittal T, McGowan E, Younson J, Kelly C, Bergmeier L A, Singh M, Lehner T. Identification of stimulating and inhibitory epitopes within the heat shock protein 70 molecule that modulate cytokine production and maturation of dendritic cells. J Immunol 174: 3306-3316 (2005)

Weis, W. I., and Drickamer, K. (1996). “Structural basis of lectin-carbohydrate recognition”. Annu. Rev. Biochem. 65: 441-473

Weis, W. I., Kahn, R., Fourme, R., Drickamer, K., and Hendrickson, W. A. (1991). “Structure of the calcium-dependent lectin domain from a rat mannose-binding protein determined by MAD phasing”. Science 254: 1608-1615

Welch W J. Heat shock proteins functioning as molecular chaperones: their roles in normal and stressed cells. Philos Trans R Soc Lond B Biol Sci 339: 327-333 (1993).

Whitehorn, E. A., Tate, E., Yanofsky, S. D., Kochersperger, L., Davis A, Mortensen, R. B., Yonkovic, S., Bell, K., Dower, W. J., and Barrett, R. W. “A generic method for expression and use of “tagged” soluble versions of cell surface receptors”. Bio/Echnology 13: 1215-1219 (1995).

Wragg, S. and Drickamer, K. (1999). “Identification of amino acid residues that determine pH dependence of ligand binding to the asialoglycoprotein receptor during endocytosis”. J. Biol. Chem. 274: 35400-35406

Wu et al., Nature Genetics, 17:141-143 (1997)

Zhang H, Wang W, Li Q, Huang W. Fusion protein of ATPase domain of Hsc70 with TRP2 acting as a tumor vaccine against B16 melanoma. Immunol Left June 15; 105(2):167-73. (2006).

Zhang, H., Robison, B., Thorgaard, G. H., and Ristow, S. S. (2000). “Cloning, mapping and genomic organization of a fish C-type lectin gene from homozygous clones of rainbow trout (Oncorhynchos Mykiss)”. Biochim. et Biophys. Acta 1494: 14-22

What is claimed is:

1. A polypeptide comprising an isolated, non-natural fragment of human HSP70 comprising QPGVLIQVYEG [SEQ ID NO:1].

2. A fusion protein comprising a trimerizing domain and at least one polypeptide that binds to QPGVLIQVYEG [SEQ ID NO: 1].

3. The fusion protein of claim 2, wherein the at least one polypeptide comprises a C-Type Lectin Like Domain (CLTD) having a loop region comprising a polypeptide sequence that binds QPGVLIQVYEG [SEQ ID NO: 1].

4. The fusion protein of claim 3 wherein a first polypeptide that binds QPGVLIQVYEG [SEQ ID NO: 1] is positioned at one of the N-terminus and the C-terminus of the timerizing domain and a second polypeptide that binds QPGVLIQVYEG [SEQ ID NO: 1] is positioned at the other of the N-terminus and the C-terminus of the trimerizing domain.

5. The fusion protein of claim 4 wherein at least one of the first and second polypeptides comprises a C-Type Lectin Like Domain (CLTD) having a loop region comprising the polypeptide sequence that binds to QPGVLIQVYEG [SEQ ID NO: 1].

6. The fusion protein of any of claims 2-5 wherein the trimerizing domain is a tetranectin trimerizing structural element.

7. A trimeric complex comprising three fusion proteins of any one of any of claims 2-5.

8. The trimeric complex of claim 7 wherein the trimerizing domain is a tetranectin trimerizing structural element.

9. A pharmaceutical composition comprising the complex of claim 8 and at least one pharmaceutically acceptable excipient.

10. A method of treating vitiligo comprising administering to a patient suffering from vitiligo the complex of claim 8.

11. A method of treating vitiligo comprising administering to a patient suffering from vitiligo the pharmaceutical composition of claim 9.

12. An isolated polynucleotide encoding a polypeptide comprising the fusion protein of claims 2-4.

13. A vector comprising the polynucleotide of claim 12.

14. A host cell comprising the vector of claim 13.

15. A method of preventing the activation of a dendritic cell by HSP70 comprising contacting tissue containing the dendritic cells and cells expressing HSP70 with the trimeric complex of claim 6.

16. A method of preventing an HSP70 related autoimmune response to stress comprising administering to a patient suffering from stress the pharmaceutical composition of claim 9.

17. A fusion protein comprising a trimerizing domain and an HSP70 polypeptide comprising QPGVLIQVYEG [SEQ ID NO: 1].

18. The fusion protein of claim 17, further comprising a C-Type Lectin Like Domain (CLTD) having a loop region comprising QPGVLIQVYEG [SEQ ID NO: 1].

19. A method of activating a dendritic cell comprising contacting the cell with the polypeptide of any of claim 1, 17 or 18.

20. A method of treating melanoma comprising administering to a patient suffering from melanoma the polypeptide of any of claim 1, 17 or 18.

21. The method of claim 20 wherein the administration is topical.

22. The method of claim 20 wherein the administration is by injection of a melanoma tumor.
